(19) United States
(12) Patent Application Publication
(10) Pub. No.: US 2023/0172846 A1
(43) Pub. Date: Jun. 8, 2023

(54) ORAL DISSOLVABLE FILM AND METHOD OF MANUFACTURING AND USING THE SAME

(71) Applicant: CURE PHARMACEUTICAL HOLDING CORP., Oxnard, CA (US)

(72) Inventor: Bhaumik PATEL, Oxnard, CA (US)

(21) Appl. No.: 17/758,215

(22) PCT Filed: Dec. 31, 2020
(86) PCT No.: PCT/US2020/067677

§ 371 Date: Jun. 29, 2022

Related U.S. Application Data
(60) Provisional application No. 62/955,484, filed on Dec. 31, 2019.

(51) Int. Cl. A61K 9/00; A61K 31/593

(52) U.S. Cl. CPC A61K 9/006; A61K 31/593

(57) ABSTRACT

The present invention provides for an oral dissolvable film and a method of manufacturing and using the same.

### CROSS-REFERENCE TO RELATED APPLICATIONS

This application is a 35 U.S.C. 371 National Stage Application of PCT/US2020/067677, filed Dec. 31, 2020, which claims the benefit of priority to U.S. Provisional Application No. 62/955,484, the disclosures of which are incorporated herein by reference in their entirety.

### BACKGROUND OF THE INVENTION

Bioavailability of orally drugs administered is relatively low. Additionally, sensitive active ingredients (e.g., sensitive to moisture, oxygen, light, pH, and/or heat) present difficulties in the selection of the suitable dosage form and the route of administration. This includes those dosage forms configured for oral administration.

Currently, there is a need for pharmaceutical formulations having an increased barrier to moisture, oxygen, light, pH, and heat to thereby confer protection to sensitive active ingredient. There is also a need for an improved bioavailability of less potent and less bioavailable active ingredients which allows the less potent active ingredients to be used at low doses. Additionally, with various pharmaceutical formulations, there is a need for an increased penetration and crossing of the mucus layer by the active ingredients, thereby allowing active ingredients to enter into systemic circulation. The above are desirable while also reducing liver/GI toxicity.

### SUMMARY OF THE INVENTION

The present invention provides for an oral dissolvable film that includes: (a) active pharmaceutical ingredient; (b) surfactant; (c) solvent for the active pharmaceutical ingredient; (d) film matrix; and (e) water; wherein, (1) when the active pharmaceutical ingredient is lipophilic or hydrophobic: (i) the surfactant is lipophilic or hydrophobic, and (ii) the solvent for the active pharmaceutical ingredient is lipophilic or hydrophobic; and (2) when the active pharmaceutical ingredient is lipophobic or hydrophilic: (i) the surfactant is lipophobic or hydrophilic, and (ii) the solvent for the active pharmaceutical ingredient is lipophobic or hydrophilic.

The present invention also provides for an oral dissolvable film that includes: (a) lipophilic active pharmaceutical ingredient; (b) oil carrier for the lipophilic active pharmaceutical ingredient; (c) self-emulsifying lipophilic surfactant for the lipophilic active pharmaceutical ingredient; (d) one or more co-surfactants; (e) one or more hydrophilic surfactants; (f) film matrix; and (g) water.

The present invention also provides for an oral dissolvable film that includes: (a) hydrophilic active pharmaceutical ingredient; (b) water carrier for the hydrophilic active pharmaceutical ingredient; (c) hydrophilic surfactant for the hydrophilic active pharmaceutical ingredient; (d) one or more co-surfactants; (e) one or more self-emulsifying surfactants; (f) film matrix; and (g) water.

The present invention also provides for a method of forming an oral dissolvable film, the method includes: (a) dissolving an active pharmaceutical ingredient in a first solvent-system to form a first mixture, wherein: (i) when the active pharmaceutical ingredient is lipophilic or hydrophobic, dissolving the active pharmaceutical ingredient in a lipophilic or hydrophobic solvent, in a lipophilic or hydrophobic surfactant, or combination thereof; or (ii) when the active pharmaceutical ingredient is hydrophilic or lipophobic, dissolving the active pharmaceutical ingredient in a hydrophilic or lipophobic solvent, in a hydrophilic or lipophobic surfactant, or combination thereof; (b) contacting the first mixture and a lipophilic or hydrophobic surfactant to form a second mixture; (c) contacting the second mixture with water and a hydrophilic or lipophobic surfactant to form a third mixture; (d) contacting the third mixture with film forming ingredient to form a slurry; and (e) casting the slurry on a substrate and curing to form the oral dissolvable film.

### DETAILED DESCRIPTION OF THE INVENTION

The present invention provides for an oral dissolvable film and a method of manufacturing and using the same.

### Definitions

The term the following terms have the meanings ascribed to them unless specified otherwise.

The words “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The oral dissolvable film described herein includes a polymeric matrix formed from an active pharmaceutical ingredient, surfactant, solvent for the active pharmaceutical ingredient, film matrix, and water. Optional additional excipients (alternatively referred to as “additives”) used to manufacture the oral film can include, e.g., one or more of: mucoadhesive polymer, plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, solvent, permeation enhancer, and preservative. Suitable excipients or additives that can be used in the formulation of oral films are described in, e.g., Lachman, et al., “The Theory and Practice of Industrial Pharmacy,” 4th Edition (2013); Rowe et al., “Handbook of Pharmaceutical Excipients,” 8th Edition (2017); and Remington, “The Science and Practice of Pharmacy,” 22nd Edition (2015). From the regulatory perspectives, all excipients and additives used in the formulation of the oral films described herein should preferably be approved for use in oral pharmaceutical dosage forms.

As used herein, the term “dissolvable film” refers to a unit dosage form which is a continuous substance, composed of pharmaceutical or food grade ingredients, relatively flat, having a discrete dimension, and configured to dissolve in liquid (e.g., those liquids present on a mucosal surface). Preferably, the dissolvable films will also be self-supporting or in other words be able to maintain their integrity and structure in the absence of a separate support. Prior to sizing to the appropriate dimension (thereby providing the unit dosage form), the dissolvable film can exist in either the unwound form (e.g., sheet) or in the wound form (e.g., bulk roll).

As administered, the dissolvable films described herein can be of any desired shape and size, provided they can effectively be administered to a mucosal surface of the body, such as the oral mucosa, tongue, eye, vagina or rectum. For example, the dissolvable films described herein can be made in the form of an article such as a strip, tape, patch, sheet, or any other suitable form known to those skilled in the art.

Specifically, dissolvable films can be relatively thin, having a thickness of from about 0.025 mm to about 0.30 mm, or they may be thicker, having thickness of from about 0.30 mm to about 0.775 mm. For some dissolvable films, the thickness may be even larger, e.g., greater than about 0.775 mm. In addition, the term “dissolvable film” includes single-layer compositions (such as single-laminated films), bilayer compositions (such as bi-laminated films), as well as multi-layer compositions (such as multi-laminated films).

The dissolvable film can effectively maintain the requisite stability of ingredients (inactive and active) present therein, over the extended periods of time typically encountered with the packaging, shipping and storage. The dissolvable film can also effectively maintain a relatively uniform distribution of such components over the extended periods of time typically encountered with the packaging, shipping and storage. From the regulatory perspectives, the dissolvable film will have no more than the permitted variance of active ingredient, per unit area of the film.

The dissolvable film can be administered to a subject (e.g., human patient) in need of a treatment of a particular disease or disorder. Selection of the active ingredient(s) within the unit dosage form described herein will be dependent upon the particular disease or disorder to be treated. The Physician's Desk Reference, 2018 Edition; The Merck Index, 15th Edition (2013); United States Pharmacopeia (USP) (2018); National Formulary as the USP-NF (2018); and the International Pharmacopoeia (Pharmacopoeia Internationalis, Ph. Int.) (2017) provide a description of the diseases or disorders that specific active ingredients have been approved for (e.g., by the U.S. FDA or EMA), in the marketing and sale of the product (e.g., within the United States or Europe). As such, a skilled artisan can look to such references for guidance in the selection of the active ingredient(s) to be present within the unit dosage form, based upon the treatment of the specific disease or disorder of particular interest (and vice-versa).

Oral dissolvable films (alternatively known as oral dissolvable films, ODFs, orally dissolving film strips, edible films, edible strips, oral film strips, oral drug strips, buccal films, sublingual films, oral soluble films, etc.) are a unit dosage form in which the dissolvable film is specifically configured for administration in the oral cavity and disintegrates over a desired period of time.

The term “oral dissolvable film” refers to a dissolvable film specifically configured for oral administration. Oral dissolvable films are composed of pharmaceutically acceptable ingredients that are edible or ingestible. The oral dissolvable film can be configured for multi- or unidirectional release. Similar in size and shape to a postage stamp, oral dissolvable films are designed for oral administration, with the user placing the strip on the tongue (enteric), under the tongue (sublingual), through the oral mucosa (mucosal), against the inside of the cheek (buccal), or on the gums (gingival). Aside from the enteric route, these drug delivery options allow the medication to bypass the first pass metabolism thereby making the medication more bioavailable. As the film dissolves, the drug can enter the blood stream enterically, mucosally, buccally, gingivally, and/or sublingually. As such, in specific embodiments the oral dissolvable film can be prepared using hydrophilic polymers that dissolves on the tongue or buccal cavity, delivering the drug to the systemic circulation via dissolution when contact with liquid is made. Oral film drug delivery accordingly uses a dissolving film to administer drugs via absorption in the mouth (buccally, sublingually, or gingivally) and/or via the small intestines (enterically). Especially for drugs which are metabolized extensively by the first-pass effect, oral films described herein provide an opportunity for a faster-acting and better absorption profile.

When systemic delivery (e.g., transmucosal delivery) is desired, the treatment site may include any area in which the adherent film described herein is capable of maintaining a desired level of pharmaceutical in the blood, lymph, or other bodily fluid. Typically, such treatment sites include the oral mucosa (e.g., tongue, under the tongue, gums, against the cheek, etc.).

When rectangular in shape, the oral dissolvable film will typically have the following two dimensional profile: length of up to about 65 mm and width of up to about 35 mm. Irrespective of shape, the oral dissolvable film will typically have a profile such that the length of its largest length, width, diameter, or cross-section is less than about 75 mm.

The oral dissolvable film will typically include a polymeric matrix formed from one or more of strip-forming polymers (e.g., mucoadhesive polymers), active pharmaceutical ingredients (APIs), and solvents. Optional additional excipients (alternatively referred to as “additives”) used to manufacture the oral film can include, e.g., one or more of plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, and preservative. Suitable excipients that can be used in the formulation of oral films are described in, e.g., Lachman, et al., “The Theory and Practice of Industrial Pharmacy,” 4^{th} Edition (2013); Rowe et al., “Handbook of Pharmaceutical Excipients,” 8th Edition (2017); and Remington, “The Science and Practice of Pharmacy,” 22nd Edition (2015). From the regulatory perspectives, all excipients used in the formulation of the oral films described herein should preferably be approved for use in oral pharmaceutical dosage forms.

The term “oral thin film” (OTF) refers to an oral dissolvable film as otherwise described herein, having specific performance characteristics and physical dimensions. Specifically, OTFs are oral dissolvable films having a thickness below about 0.400 mm (and typically below about 0.250 mm), and irrespective of the drug load, can be configured to be mucoadhesive, and are configured to dissolve and/or disintegrate very rapidly upon contact with saliva. Specifically, OTFs can disintegrate in the oral cavity (e.g., oral mucosal surface), with a relatively short in vitro disintegration time (e.g., about 120 seconds or less).

Competing forces are at play in developing OTFs. On the one hand, by virtue of being “thin,” existing OTFs typically do not have a high drug load (e.g., more than 200 mg or 40 wt. % of active ingredient). Likewise, by increasing the thickness of the OTF to increase the drug load, at some point the resulting film would no longer considered to be “thin.” Such a film is at risk of losing the aesthetic and performance characteristics of an OTF. Specifically, by increasing the thickness of the existing OTFs to support a high drug load, the resulting film may not be capable of effectively eroding, dissolving, and/or disintegrating rapidly upon contact with saliva. The resulting film may not have the requisite mucoadhesiveness desired for the film, which would allow it to “stick” and remain on the mucosal surface as it erodes. Additionally, the resulting film may not retain the requisite mechanical properties over the extended periods of time typically encountered with the packaging, shipping and storage of product. Moreover, the resulting film may not possess the capability of delivering the therapeutically effective amount of active ingredient to the subject, as intended.

The dissolvable film described herein will typically be formed from a slurry. The term “slurry” refers to a mixture of solids suspended and/or dissolved in liquid, and is suitable to be extruded, cast onto a substrate, and cured to form a dissolvable film. The solids and liquid will expectedly include those substances used to manufacture the oral dissolvable film. The solid substances employed in the manufacture of the oral dissolvable film can be dissolved and/or suspended in the liquid. The oral dissolvable film can be formed by curing the cast slurry, wherein the curing can be carried out at an elevated temperature for a period of time. In doing so, an appreciable amount of the solvent (e.g., water) will be removed.

The present invention relates to a dissolvable film that can be used to administer a desired predetermined substance, referred to herein as an “active pharmaceutical ingredient” (API) (and equivalent terms such as “active ingredient,” etc.), at an amount sufficient or effective to (1) obtain a desired result, such as the treatment of the subject, to (2) obtain a desired level of API in the subject (as evidenced by, e.g., plasma levels of the API), and/or (3) obtain a desired level of API active metabolite in the subject (as evidenced by, e.g., plasma levels of the API active metabolite).

The term “active pharmaceutical ingredient” or “active ingredient” is used to include any “drug,” “bioactive agent,” “preparation,” “medicament,” “therapeutic agent,” “physiological agent,” “nutraceutical,” or “pharmaceutical agent” and includes substances for use in the treatment of a disease or disorder. Dietary supplements, vitamins, functional foods (e.g., ginger, green tea, lutein, garlic, lycopene, capsaicin, and the like) are also included in this term.

Standard references such as, e.g., The Physician's Desk Reference, 2018 Edition; The Merck Index, 15th Edition (2013); and United States Pharmacopeia (USP) (2018) provide a description of specific active pharmaceutical ingredients, and pharmaceutically acceptable salts thereof, suitable for use with the dissolvable films described herein.

As used herein, the term “surfactant” refers to a substance that that lowers the surface tension (or interfacial tension) between two liquids, between a gas and a liquid, or between a liquid and a solid. Surfactants may act as detergents, wetting agents, emulsifiers, foaming agents, or dispersants. The surfactant can be anionic, cationic, zwitterionic, or non-ionic.

The term “solvent” refers to a substance that dissolves a solute, resulting in a solution. With the oral dissolvable film described herein, the solute can include, e.g., the film forming polymer, the active ingredient and excipients such as, e.g., plasticizer, sweetener, flavoring agent, binder, preservative, coloring agent, and pH adjusting agent. Additionally, with the oral dissolvable film described herein, the slurry can be a solution. As such, the solvent is employed to form the slurry by dissolving the desired substances to be included in the slurry (and subsequently the oral dissolvable film). The solvent can be an aqueous solvent, thereby including water. Alternatively, the solvent can include an organic liquid, such as ethanol. The water present in the oral dissolvable film described herein can function as a solvent. Additionally, the water can further optionally function as a plasticizer, process aid, or combination thereof. The term “solvent” also embraces “co-solvent,” which is a substance, present along with the solvent, that aids, facilitates, or promotes the dissolving of the solute, to provide the solution (e.g., slurry). The co-solvent will typically include an organic liquid, such as glycerin, propylene glycol, polyethylene glycol, or a combination thereof.

As used herein, the term “solvent for the active pharmaceutical ingredient” refers to a solvent as described herein, capable of specifically dissolving an active pharmaceutical ingredient.

The term “matrix,” “film matrix,” or “polymeric matrix” refers to the matrix of film forming polymer having the active ingredient embedded therein. In addition to the active ingredient, the polymeric matrix can further include additional substances embedded therein. These would include any one or more of those substances used to form the slurry. As the cast slurry is cured to provide a dissolvable film, a polymeric matrix is formed which contains the active ingredient (and optionally one or more additional substances) embedded therein. For example, when the slurry contains an active ingredient, film forming polymer, solvent, binder, and plasticizer, upon casting and curing to provide the dissolvable film, a polymeric matrix is formed which can contain each of the active ingredient, film forming polymer, solvent, binder, and plasticizer. Alternatively, the polymeric matrix can be formed containing each of the active ingredient, film forming polymer, binder, and plasticizer (i.e., no solvent).

The oral dissolvable film described herein can include a single film matrix. Alternatively, the oral dissolvable film can include multiple (e.g., 2, 3, 4, etc.) film matrices.

As used herein, the term “lipophilicity” refers to the ability of a chemical compound to dissolve in fats, oils, lipids, and non-polar solvents such as hexane or toluene. Such non-polar solvents are themselves lipophilic (translated as “fat-loving” or “fat-liking”), and the axiom that “like dissolves like” generally holds true. Thus, lipophilic substances tend to dissolve in other lipophilic substances, but hydrophilic (“water-loving”) substances tend to dissolve in water and other hydrophilic substances. Lipophilicity, hydrophobicity, and non-polarity may describe the same tendency towards participation in the London dispersion force, as the terms are often used interchangeably. However, the terms “lipophilic” and “hydrophobic” are not synonymous, as can be seen with silicones and fluorocarbons, which are hydrophobic but not lipophilic.

As used herein, the term “hydrophobicity” is the physical property of a molecule that is seemingly repelled from a mass of water (known as a hydrophobe). (Strictly speaking, there is no repulsive force involved; it is an absence of attraction.) In contrast, hydrophiles are attracted to water. Hydrophobic molecules tend to be nonpolar and, thus, prefer other neutral molecules and nonpolar solvents. Because water molecules are polar, hydrophobes do not dissolve well among them. Hydrophobic molecules in water often cluster together, forming micelles. Water on hydrophobic surfaces will exhibit a high contact angle. Examples of hydrophobic molecules include the alkanes, oils, fats, and greasy substances in general. Hydrophobic materials are used for oil removal from water, the management of oil spills, and chemical separation processes to remove non-polar substances from polar compounds. Hydrophobic is often used interchangeably with lipophilic, “fat-loving”. However, the two terms are not synonymous. While hydrophobic substances are usually lipophilic, there are exceptions, such as the silicones and fluorocarbons. The term hydrophobe comes from the Ancient Greek “having a horror of water”, constructed from Ancient Greek ‘water’, and Ancient Greek ‘fear’.

As used herein, the term “lipophobicity” also sometimes called lipophobia (from the Greek “fat” and “fear”), is a chemical property of chemical compounds which means “fat rejection”, literally “fear of fat”. Lipophobic compounds are those not soluble in lipids or other non-polar solvents. From the other point of view, they do not absorb fats. “Oleophobic” (from the Latin “oil”, Greek “oil” and “fear”) refers to the physical property of a molecule that is seemingly repelled from oil. (Strictly speaking, there is no repulsive force involved; it is an absence of attraction.) The most common lipophobic substance is water.

As used herein, the term “hydrophilicity” refers to refers to the ability of a chemical compound to dissolve in water. Such polar protic solvents are themselves hydrophilic (translated as “water-loving” or “water-liking”), and the axiom that “like dissolves like” generally holds true. Thus, hydrophilic substances tend to dissolve in water and other hydrophilic substances.

As used herein, the term “lipophilic or hydrophobic” refers to a substance that is (i) lipophilic, (ii) hydrophobic, or (iii) lipophilic and hydrophobic.

As used herein, the term “lipophobic or hydrophilic” refers to a substance that is (i) lipophobic, (ii) hydrophilic, or (iii) lipophobic and hydrophilic.

As used herein, the term “cannabinoid” refers to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids (manufactured chemically). The most notable cannabinoid is the phytocannabinoid A9-tetrahydrocannabinol (THC), the primary psychoactive compound of Cannabis. Cannabidiol (CBD) is another major constituent of the plant, representing up to 40% in extracts of the plant resin. There are at least 85 different cannabinoids isolated from Cannabis, exhibiting varied effects. The cannabinoid can be synthetically prepared (or bio-synthesized), or alternatively, can be obtained naturally (e.g., from plant matter). Either way, the cannabinoid can have the requisite purity. For example, when marketed as a nutraceutical or dietary supplement, the cannabinoid can have a purity of at least 80 wt. % pure, at least 85 wt. % pure, or at least 90 wt. % pure.

Additionally, when marketed as a pharmaceutical product, the cannabinoid can have a purity of at least 95 wt. % pure, at least 98 wt. % pure, at least 99 wt. % pure, or at least 99.5 wt. % pure).

Cannabinoids isolated from Cannabis

| | |
|---|---|
| 1. | Cannabigerol ((E)-CBG-C5) |
| 2. | Cannabigerol monomethyl ether ((E)-CBGM-C5 A) |
| 3. | Cannabinerolic acid A ((Z)-CBGA-C5 A) |
| 4. | Cannabigerovarin ((E)-CBGV-C3) |
| 5. | Cannabigerolic acid A ((E)-CBGA-C5 A) |
| 6. | Cannabigerolic acid A monomethyl ether ((E)-CBGAM-C5 A) |
| 7. | Cannabigerovarinic acid A ((E)-CBGVA-C3 A) |
| 8. | (±)-Cannabichromene (CBC-C5) |
| 9 | (+)-Cannabichromenic acid ACBCA-C5 A |
| 10. | (+)-Cannabivarichromene or (+)-Cannabichromevarin (CBCV-C3) |
| 11. | (+)-Cannabichromevarinic acid A (CBCVA-C3 A) |
| 12. | (−)-Cannabidiol (CBD-C5) |
| 13. | Cannabidiol momomethyl ether (CBDM-C5) |
| 14. | Cannabidiol-C4 (CBD-C4) |
| 15. | (−)-Cannabidivarin CBDV-C3 |
| 16. | Cannabidiorcol (CBD-C1) |
| 17. | Cannabidiolic acid (CBDA-C5) |
| 18. | Cannabidivarinic acid (CBDVA-C3) |
| 19. | Cannabinodiol (CBND-C5) |
| 20. | Cannabinodivarin (CBND-C3) |
| 21 | Δ9-Tetrahydrocannabinol (Δ9-THC-C5) |
| 22. | Δ9-Tetrahydrocannabinol-C4 (Δ9-THC-C4) |
| 23. | Δ9-Tetrahydrocannabivarin (Δ9-THCV-C3) |
| 24. | Δ9-Tetrahydrocannabiorcol (Δ9-THCO-C1) |
| 25. | Δ9-Tetrahydro-cannabinolic acid A (Δ9-THCA-C5 A) |
| 26. | Δ9-Tetrahydro-cannabinolic acid B (Δ9-THCA-C5 B) |
| 27. | Δ9-Tetrahydro-cannabinolic acid-C4A and/or B |
| | (Δ9-THCA-C4A and/or B) |
| 28. | Δ9-Tetrahydro-cannabivarinic acid A (Δ9-THCVA-C3A) |
| 29. | Δ9-Tetrahydro-cannabiorcolic acid A and/or B |
| | (Δ9-THCOA-C1A and/or B) |
| 30. | (−)-Δ8-trans-(6aR,10aR)-Δ8-Tetrahydrocannabinol (Δ8-THC-C5) |
| 31. | (−)-Δ8-trans-(6aR,10aR)-Tetrahydrocannabinolic acid A |
| | (Δ8-THCA-C5 A) |
| 32. | (−)-(6aS,10aR)-Δ9-Tetrahydrocannabinol ((−)-cis-Δ9-THC-C5) |
| 33. | Cannabinol (CBN-C5) |
| 34. | Cannabinol-C4 (CBN-C4) |
| 35. | Cannabivarin (CBN-C3) |
| 36. | Cannabinol-C2 (CBN-C2) |
| 37. | Cannabiorcol (CBN-C1) |
| 38. | Cannabinolic acid A (CBNA-C5 A) |
| 39. | Cannabinol methyl ether (CBNM-C5) |
| 40. | (−)-(9R,10R)-trans-Cannabitriol ((−)-trans-CBT-C5) |
| 41. | (+)-(9S,10S)-Cannabitriol ((+)-trans-CBT-C5) |
| 42. | (±)-(9R,10S/9S,10R)-Cannabitriol ((±)-cis-CBT-CS) |
| 43. | (−)-(9R,10R)-trans-10-O-Ethyl-cannabitriol |
| | ((−)-trans-CBT-OEt-C5) |
| 44. | (±)-(9R,10R/9S,10S)-Cannabitriol-C3 ((±)-trans-CBT-C3) |
| 45. | 8,9-Dihydroxy-Δ6a(10a)-tetrahydrocannabinol |
| | (8,9-Di-OH-CBT-C5) |
| 46. | Cannabidiolic acid A cannabitriol ester |
| | (CBDA-C5 9-OH-CBT-C5 ester) |
| 47. | (−)-(6aR,9S,10S,10aR)-9,10-Dihydroxy-hexahydrocannabinol, |
| | Cannabiripsol (Cannabiripsol-C5) |
| 48. | (−)-6a,7,10a-Trihydroxy-Δ9-tetrahydrocannabinol |
| | ((−)-Cannabitetrol) |
| 49. | 10-Oxo-Δ6a(10a)-tetrahydrocannabinol (OTHC) |
| 50. | (5aS,6S,9R,9aR)-Cannabielsoin (CBE-C5) |
| 51. | (5aS,6S,9R,9aR)-C3-Cannabielsoin (CBE-C3) |
| 52. | (5aS,6S,9R,9aR)-Cannabielsoic acid A (CBEA-C5 A) |
| 53. | (5aS,6S,9R,9aR)-Cannabielsoic acid B (CBEA-C5 B) |
| 54. | (5aS,6S,9R,9aR)-C3-Cannabielsoic acid B (CBEA-C3 B) |
| 55. | Cannabiglendol-C3 (OH-iso-HHCV-C3) |
| 56. | Dehydrocannabifuran (DCBF-C5) |
| 57. | Cannabifuran (CBF-C5) |
| 58. | (−)-Δ7-trans-(1R,3R,6R)-Isotetrahydrocannabinol |
| 59. | (±)-Δ7-1,2-cis-(1R,3R,6S/1S,3S,6R)-Isotetrahydro-cannabivarin |
| 60. | (−)-Δ7-trans-(1R,3R,6R)-Isotetrahydrocannabivarin |
| 61. | (±)-(1aS,3aR,8bR,8cR)-Cannabicyclol (CBL-C5) |
| 62. | (±)-(1aS,3aR,8bR,8cR)-Cannabicyclolic acid A (CBLA-C5 A) |
| 63. | (±)-(1aS,3aR,8bR,8cR)-Cannabicyclovarin (CBLV-C3) |
| 64. | Cannabicitran (CBT-C5) |
| 65. | Cannabichromanone (CBCN-C5) |
| 66. | Cannabichromanone-C3 (CBCN-C3) |
| 67. | Cannabicoumaronone (CBCON-C5) |
| 68. | Cannabielsoin acid A (CBEA-A) |
| 69. | 10-Ethoxy-9-hydroxy-delta-6a-tetrahydrocannabinol |
| 70. | Cannabitriolvarin (CBTV) |
| 71. | Delta-9-tetrahydrocannabiorcolic acid (THCA-C1) |
| 72. | Delta-7-cis-iso-tetrahydrocanna |
| 73. | Cannabichromanon (CBCF) |

Structure of common cannabinoids

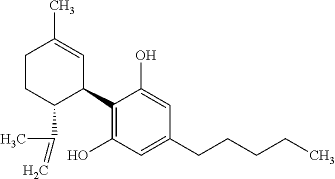 [SMILES: C=C(C)[C@@H]1CCC(C)=C[C@H]1c1c(O)cc(CCCCC)cc1O]

CBD

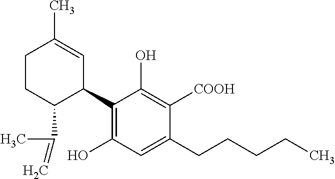 [SMILES: C=C(C)[C@@H]1CCC(C)=C[C@H]1c1c(O)cc(CCCCC)c(C(=O)O)c1O]

CBDA

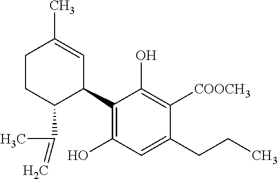 [SMILES: C=C(C)[C@@H]1CCC(C)=C[C@H]1c1c(O)cc(CCC)c(C(=O)OC)c1O]

CBDVA-OMe

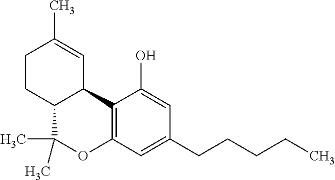 [SMILES: CCCCCc1cc(O)c2c(c1)OC(C)(C)[C@@H]1CCC(C)=C[C@@H]21]

THC

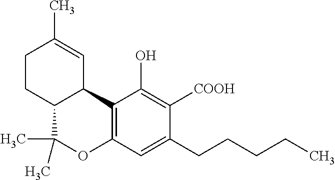 [SMILES: CCCCCc1cc2c(c(O)c1C(=O)O)[C@@H]1C=C(C)CC[C@H]1C(C)(C)O2]

THCA

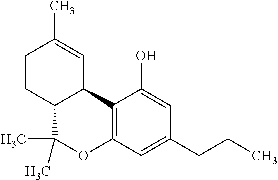 [SMILES: CCCc1cc(O)c2c(c1)OC(C)(C)[C@@H]1CCC(C)=C[C@@H]21]

THCV

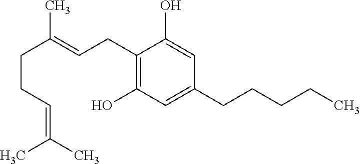 [SMILES: CCCCCc1cc(O)c(C/C=C(\C)CCC=C(C)C)c(O)c1]

CBG

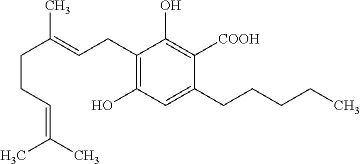 [SMILES: CCCCCc1cc(O)c(C/C=C(\C)CCC=C(C)C)c(O)c1C(=O)O]

CBGA

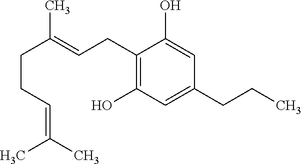 [SMILES: CCCc1cc(O)c(C/C=C(\C)CCC=C(C)C)c(O)c1]

CBGV

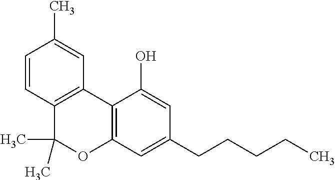 [SMILES: CCCCCc1cc(O)c2c(c1)OC(C)(C)c1ccc(C)cc1-2]

CBN

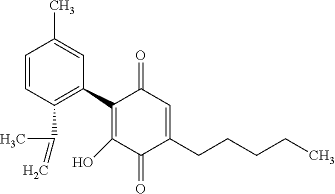

CBQ

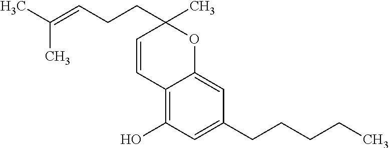 [SMILES: CCCCCc1cc(O)c2c(c1)OC(C)(CCC=C(C)C)C=C2]

CBC

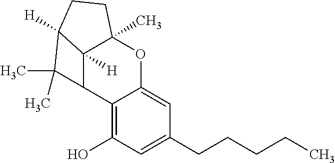

CBL

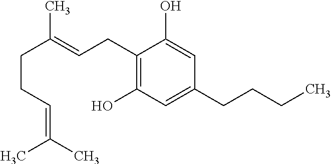 [SMILES: CCCCc1cc(O)c(C/C=C(\C)CCC=C(C)C)c(O)c1]

CBGB

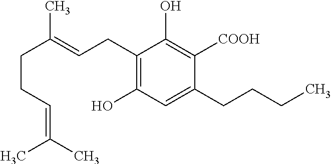 [SMILES: CCCCc1cc(O)c(C/C=C(\C)CCC=C(C)C)c(O)c1C(=O)O]

CBGBA

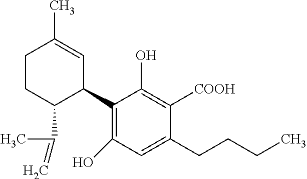 [SMILES: C=C(C)[C@@H]1CCC(C)=C[C@H]1c1c(O)cc(CCCC)c(C(=O)O)c1O]

CBDBA

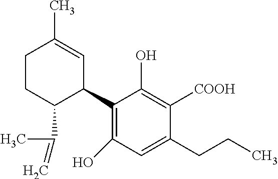 [SMILES: C=C(C)[C@@H]1CCC(C)=C[C@H]1c1c(O)cc(CCC)c(C(=O)O)c1O]

CBDVA

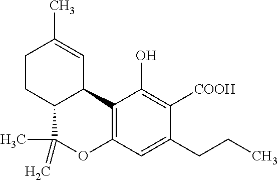

THCVA

Synthetically prepared cannabinoids, that are commercially available (e.g., Purisys™ of Athens, Ga.), are provided below.

| | | | |
|---|---|---|---|
| | | Alkyl | |
| | | Tail | |
| Common Name | Other Names | Length | CAS # |

TETRAHYDROCANNABIVARIN FAMILY

| | | | |
|---|---|---|---|
| Δ9-Tetrahydrocannabivarin | Δ9-THCV | C3 | 31262-37-0 |
| Δ8-Tetrahydrocannabivarin | Δ8-THCV | C3 | 31262-38-1 |
| Δ9-Tetrahydrocannabivarin | Δ9-THCV-NE | C3 | N/A |
| Naphtoylester | | | |
| Δ8-Tetrahydrocannabivarin | Δ8-THCV-NE | C3 | N/A |
| Naphtoylester | | | |
| Δ9-Tetrahydrocannabivarinic | Δ9-THCVA-A, | C3 | 39986-26-0 |
| Acid | Δ9-THC-VA-B | | |

| | | | |
|---|---|---|---|
| | | Alkyl | |
| | | Tail | |
| Common Name | Other Names | Length | CAS # |

CANNABIDIOLVARIN FAMILY

| | | | |
|---|---|---|---|
| (−)-Cannabidivarin | (−)-CBDV | C3 | 24274-48-4 |
| (+)-Cannabidivarin | (+)-CBDV | C3 | 1637328-94-9 |
| Cannabidivarinic Acid | CBDVA | C3 | 31932-13-S |
| Cannabidivarin Quinone | CBQV | C3 | N/A |

| | | | |
|---|---|---|---|
| | | Alkyl | |
| | | Tail | |
| Common Name | Other Names | Length | CAS # |

TETRAHYDROCANNABIBUTOL FAMILY

| | | | |
|---|---|---|---|
| Δ9-Tetrahydrocannabibutol | Δ9-THCB | C4 | 60008-00-6 |
| Δ8-Tetrahydrocannabibutol | Δ8-THCB | C4 | 51768-59-3 |
| Δ9-Tetrahydrocannabibutol | Δ9-THCB-NE | C4 | 60007-98-9 |
| Naphtoylester | | | |
| Δ8-Tetrahydrocannabibutol | Δ8-THCB-NE | C4 | N/A |
| Naphtoylester | | | |
| Δ9-Tetrahydrocannabibutolic | Δ9-THCBA-A, | C4 | 60007-98-9 |
| Acid | Δ9-THC-BA-B | | |

| | | | |
|---|---|---|---|
| | | Alkyl | |
| | | Tail | |
| Common Name | Other Names | Length | CAS # |

CANNABIDIBUTOL FAMILY

| | | | |
|---|---|---|---|
| (−)-Cannabidibutol | (−)-CBDB | C4 | 60113-11-3 |
| (+)-Cannabidibutol | (+)-CBDB | C4 | N/A |
| Cannabidibutolic Acid | CBDBA | C4 | N/A |

| | | | |
|---|---|---|---|
| | | Alkyl | |
| | | Tail | |
| Common Name | Other Names | Length | CAS # |

RARE CANNABINOIDS

| | | | |
|---|---|---|---|
| Cannabinol | CBN, USP | C5 | 521-35-7 |
| | Impurity | | |
| Cannabinolic Acid | CBNA | C5 | 2808-39-1 |
| Cannabigerol | CBG | C5 | 25654-31-3 |
| Cannabigerolic Acid | CBGA | C5 | 25555-57-1 |
| Cannabichromene | CBC | C5 | 20675-51-8 |
| Cannabichromenic Acid | CBCA | C5 | 185505-15-1 |
| Cannbicyclol | CBL | C5 | 21366-63-2 |
| Cannabicyclolic Acid | CBLA | C5 | 2283314-84-9 |
| Cannabivarin | CBNV | C3 | 33745-21-0 |
| Cannabivarinic Acid | CBNVA | C3 | 64846-02-2 |
| Cannbigerivarin | CBGV | C3 | 55824-11-8 |
| Cannabigerivarinic Acid | CBGVA | C3 | 64924-07-8 |
| Cannbichromevarin | CBCV | C3 | 57130-04-8 |
| Cannabichromevarinic Acid | CBCVA | C3 | 64898-02-8 |
| Cannabicyclolvarin | CBLV | C3 | 55870-47-8 |
| Cannabicyclolvarinic Acid | CBLVA | C3 | 2281847-63-8 |
| 3-Butylcannabinol | CBNB | C4 | 60007-99-0 |
| 3-Butylcannabinolic Acid | CBNBA | C4 | N/A |
| Cannabigerol Butyl | CBGB | C4 | N/A |
| Cannabigerol Butyric Acid | CBGBA | C4 | N/A |
| Cannabichromene Butyl | CBCB | C4 | N/A |
| Cannabichromene Buytric Acid | CBCBA | C4 | N/A |
| Cannabicyclol Butyl | CBLB | C4 | N/A |
| Cannabicyclol Butyric Acid | CBLBA | C4 | N/A |

As used herein, the term “terpene” refers to a hydrocarbon or derivative thereof, found as a natural product and biosynthesized by oligomerization of isoprene units. A terpene can be acyclic, monocyclic, bicyclic, or multicyclic. Examples include, e.g., sesquiterpenes (e.g., (−)-β-caryophyllene, humulene, vetivazulene, guaiazulene, longifolene, copaene, and patchoulol), monoterpenes (e.g., limonene and pulegone), monoterpenoids (e.g., carvone), diterpenes (e.g., taxadiene), and triterpenes (e.g., squalene, betulin, betulinic acid, lupane, lupeol, betulin-3-caffeate, allobetulin, and cholesterol). The terpene can be synthetically prepared (or bio-synthesized), or alternatively, can be obtained naturally (e.g., from plant matter). Either way, the terpene can have the requisite purity. For example, when marketed as a nutraceutical or dietary supplement, the terpene can have a purity of at least 80 wt. % pure, at least 85 wt. % pure, or at least 90 wt. % pure. Additionally, when marketed as a pharmaceutical product, the terpene can have a purity of at least 95 wt. % pure, at least 98 wt. % pure, at least 99 wt. % pure, or at least 99.5 wt. % pure).

| Terpene | Plant | Genus | Species |
|---|---|---|---|
| Myrcene | Myrtles | Myrtus | communis; nivellei; |
| | | | phyllireaefolia |
| | Cannabis | Cannabis | sativa; ruderalis; indica |
| Linalool | Mint | Mentha | spicata; arvensis; |
| | | | canadensis |
| | Lavender | Lavandula (subgenus: | spica; angustifolia; |
| | | Fabricia; Sabaudia) | latifolia; lanata; dentata, |
| | | | stoechas; pedunculata; |
| | | | viridis |
| Terpineol | Orange peel | Citrus | reticulata |
| | Junipers | Juniperus | communis; chinensis; |
| | | | conferta; rigida |
| Camphene | Chrysanthemum | Chrysanthemum | indicum |
| | Ginger | Zingiber | officinale |
| Bisabolol | Chamomile | Matricaria (or | chamomilla (or nobile) |
| | | Chamaemelum) | |
| | Figwort | Myoporum | crassifolium |
| Nerolidol | Cannabis | Cannabis | sativa; ruderalis; indica |
| Limonene | Citrus Lemon | Citrus | limon |
| Humulene | Hops | Humulus | lupulus; japonicus; |
| | | | yunnanensis |
| Terpinolene | Cannabis | Cannabis | sativa; ruderalis; indica |
| Carene | Rosemary | Salvia | rosmarinus; jordanii |
| | Cedar | Cedrus | atlantica; brevifolia; |
| | | | deodara; libani |
| Eucalyptol | Eucalyptus | Eucalyptus | obliqua |
| | Cannabis | Cannabis | sativa; ruderalis; indica |
| | Camphor laurel | Cinnamomum | camphora |
| | Bay leaves | Lauras | nobilis |
| | Wormwood | Artemisia | vulgaris |
| Ocimene | Hops | Humulus | lupulus; japonicus; |
| | | | yunnanensis |
| | Kumquats | Citrus | japonica |
| | Mango | Mangifera | indica |
| | Basil | Ocimum | basilicum |
| | bergamot orange | Citrus | × aurantium |
| Carophyllene | Peppercorn | Piper | nigrum |
| | Cloves | Syzgium | aromaticum |
| | Cannabis | Cannabis | sativa; ruderalis; indica |
| | Rosemary | Salvia | rosmarinus; jordanii |
| | Hops | Humulus | lupulus; japonicus; |
| | | | yunnanensis |
| Valencene | Nootka cypress | Callitropsis | nootkatensis |
| Geraniol | Roses | Rosa (subgenus: | persica; minutifolia; |
| | | Banksianae, Bracteatae, | stellata |
| | | Caninae, Carolinae, | |
| | | Chinensis, Gallicanae, | |
| | | Gymnocarpae, | |
| | | Laevigatae, | |
| | | Pimpinellifoliae, | |
| | | Synstylae) | |
| | Wine grapes | Vitis | vinifera |
| Borneol | Borneo camphor | Dryobalanops | aromatica |
| | Ngai camphor; | Blumea | balsamifera |
| | sambong | | |
| Pulegone | Catnip | Nepeta | cataria |
| | Peppermint | Mentha | piperita |
| | Pennyroyal | Hedeoma | pulegioides |
| Guaiazulene | Chamomile | Matricaria (or | chamomilla (or nobile) |
| | | Chamaemelum) | |
| | Guaiacum tree | Guaiacum | sanctum, angustifolium, |
| | | | coulteri, officinale |
| Lupeol | Lupine seed | Lupinus | luteus |
| Lupane | Lupine seed | Lupinus | luteus |
| Betulin | Brich tree | Betula (Subgenus: | alleghaniensis, cordifolia, |
| Betulinic acid | | Betulenta, Betulaster, | glandulosa, lenta, |
| Lupeol | | Neurobetula, | michauxii, minor, nana, |
| | | Chamaebetula) | neoalaskana, nigra, |
| | | | occidentalis, papyrifera, |
| | | | populifolia, pumila, uber |
| Squalene | Amaranth seed | Amaranthus (subgenus: | acanthochiton, |
| | | Acnida; Albersia) | acutilobus, albus, |
| | | | anderssonii, californicus |
| | Wheat germ | Triticum | aestivum |
| | Olive | Olea | europaea |
| Carvone | Caraway seed | Carum | carvi |
| | Spearmint | Mentha | spicata |
| | Dill | Anethum | graveolens |
| Patchoulol | Patchouli | Pogostemon | cablin |
| Copaene | Copaiba tree | Copaifera | langsdorfii |
| Longifolene | Pine | Pinus | longifolia |
| Pinene | Pine | Pinus (subgenus: | densata, densiflora, |
| | | Strobus; Pinus) | pinea, sylvestris |
| Vetivazulene | Vetiver | Chrysopogon | zizanioides |
| Nerol | Lemon Grass | Cymbopogon | nardus; citratus; |
| | | | flexuosus; martinii, |
| | | | schoenanthus |

Synthetically prepared terpenes, which are commercially available (e.g., Purisys™ of Athens, Ga.), are provided below.

| Terpene | CAS# |
|---|---|
| Alpha-Pinene | 51634232009 |
| Beta-Pinene | 51634232109 |
| Beta-Myrcene | 51634232209 |
| Alpha-Terpinene | 51634232309 |
| Limonene | 51634232409 |
| Beta-Ocimene | 51634232509 |
| Terpinolene | 51634232609 |
| Linalool | 51634232709 |
| Fenchyl Alcohol | 51634232809 |
| Borneol Isomers | 51634232909 |
| Alpha-Terpineol | 51634233009 |
| Trans-caryophyllene | 51634233109 |
| Alpha-humulene | 51634233209 |
| Trans-nerolidol | 51634233309 |
| Guaiol | 51634233409 |
| Alpha-Bisabolol | 51634233509 |

As used herein, the term “flavonoid” refers to ubiquitous plant natural products with various polyphenolic structures. Flavonoids can be extracted from fruits, vegetables, grains, bark-, roots, stems, flowers, and teas or can be biosynthetically produced. The role of flavonoids in plants includes UV protection, aid in plant growth, defense against plaques, and provide the color and aroma of flowers.

Flavonoids can be divided into classes (e.g., anthocyanin, chalcone, flavone, flavonol, isoflavone, and flavonone) and subclasses depending on the carbon of the C ring on which the B ring is attached and the degree of unsaturation and oxidation of the C ring.

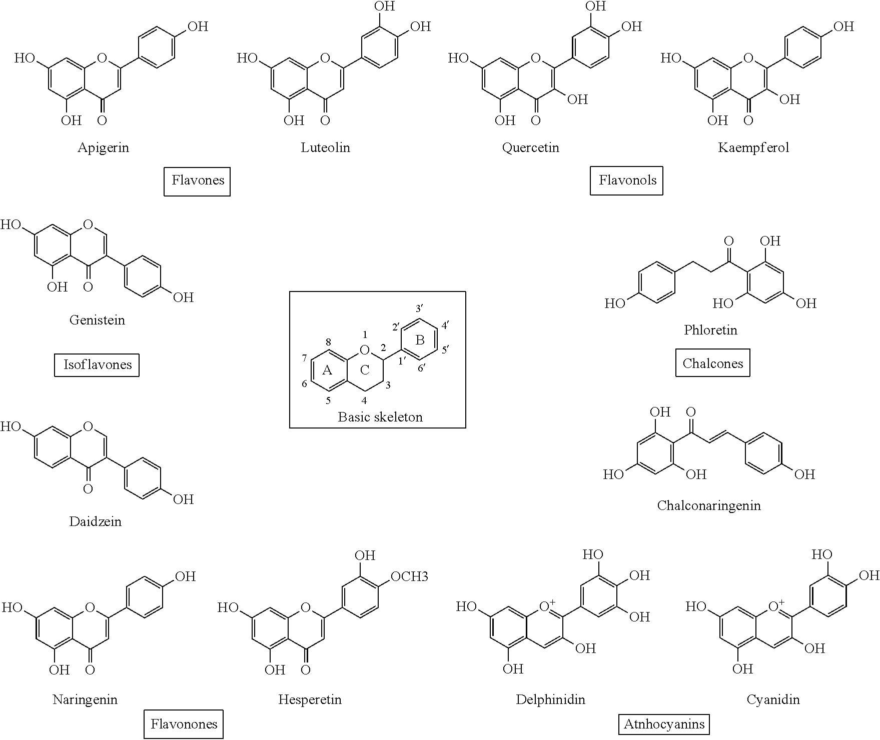

| | | | Examples of |
|---|---|---|---|
| Flavonoid classes | Subclasses | Natural sources | natural sources |
| Anthocyanins | Cyanidin, Malvidin, | Fruits, vegetables, | Cranberries, plums, |
| | Delphinidin, Peonidin | nuts, dried fruits, | cherries, sweet |
| | | medicinal plants | potatoes, black |
| | | | currants, red |
| | | | grapes, merlot |
| | | | grapes, raspberries, |
| | | | strawberries, |
| | | | blueberries, |
| | | | bilberries and |
| | | | blackberries |
| Chalcones | Phloretin, Arbutin, | Fruits, vegetables, | Tomatoes, pears, |
| | Phlioridzin | medicinal plants | strawberries, |
| | | | bearberries and |
| | | | certain wheat |
| | | | products |
| Flavonones | Hesperitin, Naringin, | Fruits (citrus), | Oranges, lemons, |
| | Naringenin, Eriodictyol, | medicinal plants | grapes, rosehips |
| | Hesperidin | | |
| Flavones | Apigenin, Tangeretin, | Fruits, medicinal | Celery, parsley, red |
| | Baicalein, Rpoifolin | plants | peppers, |
| | | | chamomile, mint, |
| | | | ginkgo biloba, |
| | | | broccoli, green |
| | | | pepper, thyme, |
| | | | dandelion, perilla, |
| | | | tea, carrot, |
| | | | rosemary, oregano, |
| | | | Cannabis sativa |
| Flavonols | Quercetin, Myricetin, | Fruits, vegetables, | Onion, kale, |
| | Rutin, Morin, | medicine plants | lettuce, tomatoes, |
| | Kaempferol | | apples, grapes, |
| | | | berries, tea, red |
| | | | wine, broccoli, |
| | | | potatoes, brussel |
| | | | sprouts, squash, |
| | | | cucumbers, lettuce, |
| | | | green beans, |
| | | | spinach, peaches, |
| | | | blackberries |
| Isoflavonoids | Genistin, Genistein, | Legumes, | Soybeans, lupin, |
| | Daidzein, Glycetein, | medicinal plants | fava beans, kudzu, |
| | Daidzin | | psoralea, red |
| | | | clover, alfalfa |
| | | | sprouts, peanuts, |
| | | | chickpeas |

| Flavonoid | |
|---|---|
| classes | Structure of flavonoid classes |
| Anthocyanins | Double bonds between positions 1 and 2, 3 and 4 of the |
| | C ring; Hydroxyl groups at positions 5 and 7 in the A |
| | ring and 3′, 4′ and/or 5′ of the B ring; Methylation or |
| | acylation at the hydroxyl groups on the A and B |
| | rings vary |
| Chalcones | Absence of ‘C ring’ of the basic flavonoid skeleton |
| | structure |
| Flavonones | C ring is saturated (contains no double bonds) |
| Flavones | Double bond between positions 2 and 3 and a ketone in |
| | position 4 of the C ring; Most have a hydroxyl group in |
| | position 5 or 7 of the A ring of the A ring or 3′ and 4′ |
| | of the B ring (varies according to the taxonomic |
| | classification of the particular plant) |
| Flavonols | Double bond between positions 2 and 3, a ketone in |
| | position 4 and hydroxyl group in position 3 of the C |
| | ring; the ketone group the C ring may also be |
| | glycosylated; very diverse in methylation and |
| | hydroxylation patterns |
| Isoflavonoids | B ring is attached to the 3 position of the C ring and |
| | contains a hydroxyl group at the 4′ position; |
| | hydroxylation of the A ring varies |

Studies on flavonoids have revealed an increasing number of health benefits showing anti-oxidant, anti-inflammatory, anti-mutagenic, and anti-carcinogenic properties by inhibiting numerous pro-inflammatory and pro-oxidative enzymes (e.g., xanthine oxidase (XO), cyclo-oxygenase (COS), lipoxygenase, phosphoinositide 3-kinase, and acetylcholinesterase). This may have benefits towards numerous diseases and medical conditions (e.g., pain, cancer, arthersclerosis, Alzheimer's disease). There is a growing interest in the medicinal properties of Cannabis (Cannabis sativa, Cannabis indica, Cannabis ruderalis). Studies have shown that Cannaflavin A and Cannflavin B, prenylated flavones, have anti-inflammatory properties greater than aspirin. Cannflavin A and B can be isolated from Cannabis sativa and biosynthesized. Recent reports have shown that the flavonoid FBL-03G has shown to increase survival rate of subjects suffering from pancreatic cancer.

Synthetically prepared flavonoids, which are commercially available (e.g., Cannflavin B from Toronto Research Chemicals), are provided below.

| Flavonoid | CAS# |
|---|---|
| Cannflavin A | 76735-57-4 |
| Cannflavin B | 76735-58-5 |
| Myricetin | 529-44-2 |
| (−)-Epigallocathechin gallate | 989-51-5 |
| Polyphenon 60 from green tea | 138988-88-2 |
| (−)-Gallocathechin | 3371-27-5 |
| Kaempferol | 520-18-3 |
| (±)-Catechin hydrate | 7295-85-4 (anhydrous) |
| Galangin | 548-83-4 |
| Hesperidin | 520-26-3 |
| Baicalein | 491-67-8 |
| Icariin | 489-32-7 |
| Orientin | 28608-75-5 |
| Liquiritigenin | 578-86-9 |
| Acacetin | 480-44-4 |
| Diosmetin | 520-34-3 |
| Scutellarein | 529-53-3 |
| Luteolin | 491-70-3 |

The flavonoid can be synthetically prepared, or alternatively, can be obtained naturally (e.g., from plant matter). Either way, the flavonoid can have the requisite purity (e.g., at least 95 wt. % pure, at least 98 wt. % pure, at least 99 wt. % pure, or at least 99.5 wt. % pure).

As used herein, the term “pharmaceutically acceptable” refers to those compounds, counterions, salts, excipients, active ingredients, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. This would include, e.g., those substances present on the FDA's Inactive Ingredient Database (IID) (https://www.accessdata.fda.gov/scripts/cder/iig/index.Cfm) as well as those substances considered to be generally recognized as safe (GRAS).

As used herein, the term “psychedelic agent” or “psychedelics” refers to a hallucinogenic class of psychoactive drug whose primary effect is to trigger non-ordinary states of consciousness (known as psychedelic experiences or “trips”) via serotonin 2A receptor agonism. This causes specific psychological, visual and auditory changes, and often a substantially altered state of consciousness. “Classic” psychedelic drugs include mescaline, LSD, psilocybin, and DMT. Most psychedelic drugs fall into one of the three families of chemical compounds: tryptamines, phenethylamines, or lysergamides. These chemicals all activate serotonin 5-HT2A receptors, which modulate the activity of key circuits in the brain involved with sensory perception and cognition, however the exact nature of how psychedelics induce changes in perception and cognition through the 5-HT2A receptor is still unknown. The psychedelic experience is often compared to non-ordinary forms of consciousness such as those experienced in meditation, mystical experiences, and near-death experiences. The phenomenon of ego dissolution is often described as a key feature of the psychedelic experience. Examples include:
LSD (Lysergic acid diethylamide, a.k.a. acid) is made from a substance found in ergot, which is a fungus that infects rye.
Psilocin is a naturally occurring substance found in psilocybin mushrooms and is found in many parts of the world.
Mescaline is derived from the Mexican peyote and San Pedro cactus and produces similar effects to LSD.
DMT (Dimethyltryptamine) is structurally similar to psilocin, an alkaloid found in psilocybin mushrooms. It can be synthesized in the laboratory but is also a naturally occurring component of several plants.
DOM is a member of the DOx family of compounds which are known for their high potency, long duration, and mixture of psychedelic and stimulant effects.
2C-B (4-Bromo-2,5-dimethoxyphenethylamine) is a psychedelic drug first synthesized in 1974. 2C-B is considered both a psychedelic and a mild entactogenic. ‘Entactogen’ means ‘touching within’ and is a term used by psychiatrists to classify MDMA and related drugs.
Peyote (Lophophora williamsii) is the most well-known and potent psychedelic cactus, although the smallest and slowest growing. Instead of growing upward to form a column, it grows as ‘buttons’ low to the ground. It has been used by Native Americans for over 5000 years.
25-NBOMe (N-methoxybenzyl) is the name for a series of drugs that have psychedelics effects. Reports indicate that there are a number of different versions of NBOMe available—all with differing effects.
Ecstasy (alternatively known as Molly or MDMA) is 3,4-methylenedioxymethamphetamine. It is a laboratory-made drug that produces a “high” similar to the stimulants called amphetamines. It also produces psychedelic effects, similar to the hallucinogens mescaline and LSD.

The term “unit dosage” or “unit dosage form” refers to an oral dissolvable film sized to the appropriate dimension, such that the individual film contains a desired amount of active ingredient. Prior to sizing to the appropriate dimension (thereby providing the unit dosage form), the dissolvable film can exist in either the unwound form (e.g., sheet) or in the wound form (e.g., bulk roll).

The term “plasticizer” refers to a substance that, when added to polymer(s), they make the polymer more pliable and softer, enhancing the flexibility and plasticity of the films while reducing the brittleness. The plasticizer is believed to permeate the polymer structure, disrupting intermolecular hydrogen bonding, and permanently lowers intermolecular attractions. Plasticizers can be used to allow initial film forming, to reduce the brittleness, and improve the processability and flexibility of the resulting film, thereby avoiding cracking, e.g., during the curing process. Suitable plasticizers include, e.g., glycerin, water, polyethylene glycol, honey, propylene glycol, monoacetin, triacetin, triethyl citrate, sorbitol, 1,3-butanediol, D-glucono-1,5-lactone, diethylene glycol, castor oil, and combinations thereof.

As used herein, the term “antimicrobial agent” refers to an agent that kills microorganisms or stops their growth.

As used herein, the term “self-emulsifying” refers to the ability of an dissolvable film described herein, to form an emulsion after contact with an oral mucosal surface (e.g., when placed in the oral cavity), for oral (PO) administration, buccal administration, sublingual administration, enteral administration, or gingival administration. The emulsion can be formed, e.g., within 120, 90, 60, or 30 seconds after contact with an oral mucosal surface.

As used herein, the term “subject” refers to living organisms such as humans, dogs, cats, and other mammals. Administration of the medicaments included in the oral dissolvable films of the present invention can be carried out at dosages and for periods of time effective for the treatment of the subject. In some embodiments, the subject is a human. Unless otherwise specified, the human subject can be a male or female, and can further be an adult, adolescent, child, toddler, or infant.

The term “particle-size distribution” or “PSD” refers to a list of values or a mathematical function that defines the relative amount, typically by mass, of particles present according to size. For example, the mass-median-diameter (MMD) (expressed as, e.g., d10, d50, d90, etc.) refers to the log-normal distribution mass median diameter. The MMD is considered to be the average particle diameter by mass. The particle size distribution can be obtained with a Malvern Mastersizer.

Particle size Distribution D10 (or d10) is also written as X10, D(0,1) or X(0,1). It represents the particle diameter corresponding to 10% cumulative (from 0 to 100%) undersize particle size distribution. In other words, if particle size D10 is 7.8 um, then 10% of the particles in the tested sample are smaller than 7.8 micrometer, or the percentage of particles smaller than 7.8 micrometer is 10%. D10 is a typical point in particle size distribution analysis. D10 is also divided into Dv10, Dw10 and Dn10. Dv10 means volume D10, whereas Dw10 is mass D10 and Dn10 is number D10.

Particle size Distribution D50 (or d50) is also written as X50, D(0,5) or X(0,5). It represents the particle diameter corresponding to 50% cumulative (from 0 to 100%) undersize particle size distribution. In other words, if particle size D50 is 7.8 um, then 50% of the particles in the tested sample are smaller than 7.8 micrometer, or the percentage of particles smaller than 7.8 micrometer is 50%. D50 is a typical point in particle size distribution analysis. D50 is also divided into Dv50, Dw50 and Dn50. Dv50 means volume D50, whereas Dw50 is mass D50 and Dn50 is number D50.

Particle size Distribution D90 (or d90) is also written as X90, D(0,9) or X(0,9). It represents the particle diameter corresponding to 90% cumulative (from 0 to 100%) undersize particle size distribution. In other words, if particle size D90 is 7.8 um, then 90% of the particles in the tested sample are smaller than 7.8 micrometer, or the percentage of particles smaller than 7.8 micrometer is 90%. D90 is a typical point in particle size distribution analysis. D90 is also divided into Dv90, Dw90 and Dn90. Dv90 means volume D90, whereas Dw90 is mass D90 and Dn90 is number D90.

As used herein, the term “mucous membrane” (and related “mucosa” and “mucosal surface”) refers to a membrane that lines various cavities in the body or covers those surfaces. It consists of one or more layers of epithelial cells overlying a layer of loose connective tissue. It is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated. Mucosal surfaces specifically include, e.g., oral mucosa, tongue, vaginal mucosa, nasal mucosa, and the anal canal.

As used herein, the term “transmucosal,” as used herein, refers to any route of administration via a mucosal membrane or mucosal surface. Examples include, but are not limited to, buccal, sublingual, nasal, vaginal, and rectal.

As used herein, the term “buccal administration” refers to a topical route of administration by which a drug held or applied in the buccal area (in the cheek) diffuses through the oral mucosa (tissues which line the mouth) and enters directly into the bloodstream. Buccal administration may provide better bioavailability of some drugs and a more rapid onset of action compared to oral administration because the medication does not pass through the digestive system and thereby avoids first pass metabolism. Liver and GI toxicities may also be avoided.

As used herein, the term “buccal space” (also termed the buccinator space) refers to a fascial space of the head and neck (sometimes also termed fascial tissue spaces or tissue spaces). It is a potential space in the cheek and is paired on each side. The buccal space is superficial to the buccinator muscle and deep to the platysma muscle and the skin. The buccal space is part of the subcutaneous space, which is continuous from head to toe.

As used herein, the term “oral cavity” or “mouth” or “buccal cavity” refers to the opening through which many animals take in food and issue vocal sounds. It is also the cavity lying at the upper end of the alimentary canal, bounded on the outside by the lips and inside by the pharynx and containing in higher vertebrates the tongue and teeth. In human anatomy, the mouth is the first portion of the alimentary canal that receives food and produces saliva. The oral mucosa is the mucous membrane epithelium lining the inside of the mouth. The mouth consists of two regions, the vestibule and the oral cavity proper. The mouth, normally moist, is lined with a mucous membrane, and contains the teeth. The lips mark the transition from mucous membrane to skin, which covers most of the body.

As used herein, the term “oral mucosa” refers to the mucous membrane lining the inside of the mouth and consists of stratified squamous epithelium termed oral epithelium and an underlying connective tissue termed lamina propria. Oral mucosa can be divided into three main categories based on function and histology: (1) Masticatory mucosa, keratinized stratified squamous epithelium, found on the dorsum of the tongue, hard palate and attached gingiva; (2) Lining mucosa, nonkeratinized stratified squamous epithelium, found almost everywhere else in the oral cavity, including the: (a) Buccal mucosa refers to the inside lining of the cheeks and floor of the mouth and is part of the lining mucosa; (b) Labial mucosa refers to the inside lining of the lips and is part of the lining mucosa; and (c) Alveolar mucosa refers to the lining between the buccal and labial mucosae. It is a brighter red, smooth and shiny with many blood vessels, and is not connected to underlying tissue by rete pegs; and (3) Specialized mucosa, specifically in the regions of the taste buds on lingual papillae on the dorsal surface of the tongue that contains nerve endings for general sensory reception and taste perception.

As used herein, the term “oral mucosal surface” refers to a surface of the oral mucosa.

As used herein, the term “sublingual administration,” from the Latin for “under the tongue,” refers to the pharmacological route of administration by which substances diffuse into the blood through tissues under the tongue. When a drug comes in contact with the mucous membrane beneath the tongue, it is absorbed. Because the connective tissue beneath the epithelium contains a profusion of capillaries, the substance then diffuses into them and enters the venous circulation. In contrast, substances absorbed in the intestines are subject to first-pass metabolism in the liver before entering the general circulation. Sublingual administration has certain advantages over oral administration. Being more direct, it is often faster, and it ensures that the substance will risk degradation only by salivary enzymes before entering the bloodstream, whereas orally administered drugs must survive passage through the hostile environment of the gastrointestinal tract, which risks degrading them, by either stomach acid or bile, or by enzymes such as monoamine oxidase (MAO). Furthermore, after absorption from the gastrointestinal tract, such drugs must pass to the liver, where they may be extensively altered; this is known as the first pass effect of drug metabolism. Due to the digestive activity of the stomach and intestines, the oral route is unsuitable for certain substances.

As used herein, the term “gingival administration” refers to the pharmacological route of administration by which substances diffuse into the blood through tissues in the gums. The gums or gingiva (plural: gingivae), consist of the mucosal tissue that lies over the mandible and maxilla inside the mouth.

As used herein, the term “enteral administration” refers to a drug administration via the human gastrointestinal tract. Enteral administration involves the esophagus, stomach, and small and large intestines (i.e., the gastrointestinal tract). Methods of administration include oral and rectal. Enteral administration may be divided into three different categories, depending on the entrance point into the GI tract: oral (by mouth), gastric (through the stomach), and rectal (from the rectum). (Gastric introduction involves the use of a tube through the nasal passage (NG tube) or a tube in the belly leading directly to the stomach (PEG tube). Rectal administration usually involves rectal suppositories.) Enteral medications come in various forms, including, e.g., tablets to swallow, chew or dissolve in water; capsules and chewable capsules (with a coating that dissolves in the stomach or bowel to release the medication there), oral soluble films, time-release or sustained-release tablets and capsules (which release the medication gradually), osmotic delivery systems, powders or granules, and liquid medications or syrups.

As used herein, the term “oral administration” or “PO” refers to a route of administration where a substance is taken through the mouth. Many medications are taken orally because they are intended to have a systemic effect, reaching different parts of the body via the bloodstream.

As used herein, the term “moisture content” refers to the quantity of water contained in a dissolvable firm described herein. The moisture content can encompass bound water and unbound water. Water content is expressed as a ratio, which can range from 0 (completely dry) to the value of the dissolvable film's porosity at saturation. It can be given on a volumetric or mass (gravimetric) basis. Typically, the moisture content will be expressed as a weight percent (e.g., 10 wt. %).

Water content can be directly measured using a drying oven. Other methods that determine water content of a sample include chemical titrations (for example the Karl Fischer titration), determining mass loss on heating (perhaps in the presence of an inert gas), or after freeze drying. The Dean-Stark method is also commonly used. Unless specified otherwise, the loss on drying (LOD) method can be employed to calculate the moisture content of a dissolvable film described herein.

As used herein, the term “disintegration” refers to a substance (e.g., matrix of an oral dissolvable film) breaking up or falling apart. The substance will lose cohesion or strength and can fragment into pieces. When placed in the mouth, the substance will break apart in the saliva.

As used herein, the term “bioavailability” refers to a subcategory of absorption and is the fraction (%) of an administered drug that reaches the systemic circulation. When a medication is administered intravenously, its bioavailability is 100%. However, when a medication is administered via routes other than intravenous, its bioavailability is generally lower than that of intravenous due to intestinal endothelium absorption and first-pass metabolism. Thereby, mathematically, bioavailability equals the ratio of comparing the area under the plasma drug concentration curve versus time (AUC) for the extravascular formulation to the AUC for the intravascular formulation. AUC is utilized because AUC is proportional to the dose that has entered the systemic circulation.

As used herein, the term “dissolution” refers to a substance (e.g., active ingredient or matrix of an oral dissolvable film) dissolving or being dissolved. When placed in the mouth, the substance will dissolve in saliva.

The term “effective amount” is used herein to generally include an amount of active ingredient present in the oral dissolvable film, effective for treating or preventing a disease, disorder, or condition in a subject, as described herein.

The term “treating” with regard to a subject, refers to improving at least one symptom of the subject's disease, disorder, or condition. Treating includes curing, improving, or at least partially ameliorating the disease, disorder, or condition, or any of the symptoms thereof.

As used herein, “pharmacokinetics,” sometimes abbreviated as “PK” refers to a branch of pharmacology dedicated to determining the fate of substances administered to a living organism. It attempts to analyze chemical metabolism and to discover the fate of a chemical from the moment that it is administered up to the point at which it is completely eliminated from the body. Pharmacokinetics is the study of how an organism affects a drug, whereas pharmacodynamics (PD) is the study of how the drug affects the organism. Both together influence dosing, benefit, and adverse effects, as seen in PK/PD models.

PK therefore refers to the study of the uptake of drugs by the body, the biotransformation they undergo, the distribution of the drugs and their metabolites in the tissues, and the elimination of the drugs and their metabolites from the body over a period of time.

The following are commonly measured pharmacokinetic metrics:

### Pharmacokinetic Metrics

| Characteristic | Description |
|---|---|
| Dose | Amount of drug administered. |
| Dosing interval | Time between drug dose administrations. |
| Cmax | The peak plasma concentration of a drug after |
| | administration. |
| Tmax | Time to reach Cmax. |
| Cmin | The lowest (trough) concentration that a drug |
| | reaches before the next dose is administered. |
| Volume of | The apparent volume in which a drug is distributed |
| distribution | (i.e., the parameter relating drug concentration in |
| | plasma to drug amount in the body). |
| Concentration | Amount of drug in a given volume of plasma. |
| Absorption | The time required for the concentration of the drug |
| half life | to double its original value for oral and other |
| | extravascular routes. |
| Absorption rate | The rate at which a drug enters into the body for |
| constant | oral and other extravascular routes. |
| Elimination | The time required for the concentration of the drug |
| half-life | to reach half of its original value. |
| Elimination rate | The rate at which a drug is removed from the body. |
| constant | |
| Infusion rate | Rate of infusion required to balance elimination. |
| Area under the | The integral of the concentration-time curve (after |
| curve | a single dose or in steady state). |
| Clearance | The volume of plasma cleared of the drug per unit |
| | time. |
| Bioavailability | The systemically available fraction of a drug. |
| Fluctuation | Peak trough fluctuation within one dosing interval |
| | at steady state. |

As used herein, the term “substrate” refers to a base object in which the slurry is cured onto. Once coated with the slurry, the substrate typically proceeds through the dryer where the slurry is at least partially cured. Typically, a roll of substrate is placed in the unwinding station and tension is applied to the line. Any suitable substrate can be used, such as, e.g., Polyethylene Terephthalate (PET) or siliconized paper. PET is a thermoplastic polymer resin of the polyester family used as the substrate when coating and drying the product. Likewise, siliconized paper is a stable, release paper manufactured with two sides of polyethylene and coated with silicon polymer on one side used as the substrate when coating and drying the product.

As used herein, the term “curing” refers to the chemical process that produces a dissolvable film (as described herein) from a slurry (also described herein). The process can be carried out by removing solvent (water), by toughening or hardening of polymer material present in the slurry, by cross-linking the polymer chains, etc. The term curing can be used to refer to the processes where starting from a liquid (or semi-solid) solution (e.g., slurry), a solid product (e.g., dissolvable film) is obtained. Curing can be initiated by heat, radiation, electron beams, or chemical additives. To quote from IUPAC: curing “might or might not require mixing with a chemical curing agent.” IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook. Thus, two broad classes are (i) curing induced by chemical additives (also called curing agents, hardeners) and (ii) curing in the absence of additives. An intermediate case involves a mixture of resin and additives that requires external stimulus (light, heat, radiation) to induce curing.

As used herein, the term “mucoadhesive agent” refers to a substance that, upon contact with a mucosal surface (e.g., oral cavity), will adhere therein. The mucoadhesive agent, when placed in the oral cavity in contact with the mucosa therein, will adhere to the mucosa. The mucoadhesive agent permits a close and extended contact of the composition of the oral dissolvable film with the mucosal surface of the subject, by promoting adherence of the composition to the mucosa, and facilitating the release of the active ingredient from the composition. The mucoadhesive agent can be a polymeric compound, such as a cellulose derivative but it can be also a natural gum, alginate, pectin, or such similar polymer. The concentration of the mucoadhesive agent can be adjusted to vary the length of time that the film adheres to the mucosa or to vary the adhesive forces generated between the film and mucosa. Mucoadhesive agents include, e.g., carboxymethyl cellulose (CMC), carboxymethyl cellulose sodium (CMC-Na), polyvinyl alcohol, polyvinyl pyrrolidone (povidone), sodium alginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl acetate, dimethylpolysiloxanes, polyoxyalkylene block copolymers, pectin, chitosan, carrageenan, xanthan gum, gellan gum, gum Arabic, locust bean gum, and hydroxyethylmethacrylate copolymers.

As used herein, the term “binder” refers to a substance, typically a polymer, used to hold the ingredients together. Binders ensure that the oral dissolvable films can be formed with the requisite mechanical strength. The binders also provide the requisite volume to low amount of active present in dissolvable films. The presence of the binder also facilitates the formation of the cured film. As such, the binder includes those substances, which when present in the cast slurry and upon curing, will effectively provide for a cured film. The binder may also be referred to as a “film forming agent,” or more specifically a “film forming polymer” when it is a polymer. The polymer can be a natural polymer or a synthetic polymer. Natural polymers include, e.g., pullulan, sodium alginate (Na alginate), pectin, gelatin, chitosan, and maltodextrin. Synthetic polymers include, e.g., hydroxpropyl cellulose (HPC), hydroxpropyl methylcellulose (HPMC), carboxymethyl cellulose (CMC), sodium carboxymethylcellulose (CMC-Na), microcrystalline cellulose (MCC), polyvinyl alcohol (PVA), polyethylene oxide (PEO), polyvinylpyrrolidone (PVP), and Kollicoat® (e.g., Kollicoat® Protect or Kollicoat® IR).

As used herein, the term “filler” or “bulking agent” refer to substances that add bulk to the pharmaceutical dosage form, making very small active ingredient components easy for consumer to take. Fillers are added to pharmaceutical dosage form to help with the manufacturing and stabilization of these products. Fillers bind and stabilize the dosage form. They do not alter or impact the effectiveness of the active pharmaceutical ingredient (API). Examples include: lactose, glucose, plant cellulose, microcrystalline cellulose (MCC), and calcium carbonate.

As used herein, the term “saliva stimulating agent” or “salivary stimulant” refers to a substance capable of increasing the production of saliva, thereby increasing salivary flow rate. Suitable saliva stimulating agents include organic acids (e.g., ascorbic acid and malic acid), parasympathomimetic drugs (e.g., choline esters such as pilocarpine hydrochloride and cholinesterase inhibitors), physostigmine, and other substances (e.g., xylitol, xylitol/sorbitol, and nicotinamide).

As used herein, the term “stabilizing and thickening agent” or “gelling agent” refers to substances employed to improve the viscosity and consistency of the slurry before casting. Active ingredient content uniformity is often a requirement for all dosage forms, particularly those containing low dose highly potent active ingredients. To uniquely meet this requirement, oral dissolvable film formulations can contain uniform dispersions of active ingredient throughout the whole manufacturing process. Examples of stabilizing and thickening agents include, e.g., alginic acid, sodium alginate, potassium alginate, ammonium alginate, calcium alginate, agar, carrageenan, locust bean gum, pectin, and gelatin.

As used herein, the term “flavoring agent” refers to a substance used to impart a flavor, e.g., to improve the attractiveness and acceptance by the subject. The basic taste sensations are salty, sweet, bitter, sour, and umami. Flavors may be chosen from natural and synthetic flavorings. An illustrative list of such agents includes volatile oils, synthetic flavor oils, flavoring aromatics, oils, liquids, oleoresins or extracts derived from plants, leaves, flowers, fruits, stems and combinations thereof. The flavoring agent can include, e.g., one or more of honey, anise, cherry, mint, peppermint, spearmint, menthol, levomenthol, watermint, gingermint, lemongrass, cardamom, sage, cinnamon, ginger, allspice, clove, eugenol, orange, wintergreen, lemon, lime, tangerine, ginger, and nutmeg. The flavoring agent can be available as a solid (e.g., powder), as a liquid (e.g., oil), or a combination thereof.

As used herein, the term “taste masking agent” refers to a substance used to mask the unpleasant taste of a substance present in the formulation, to improve the attractiveness and acceptance by the subject. For example, the taste masking agent can refer to a substance used to mask the bitter taste of the active ingredient. With the oral dissolvable films described herein, the taste masking agent can include, e.g., at least one of honey, anise, mint, peppermint, cinnamon, magna sweet, citrus, and fruit (e.g., cherry). In addition to imparting a flavor, the flavoring agent can optionally also mask the taste of any unpleasant or bitter tasting substances (e.g., the active ingredient) present in the oral dissolvable film. In such embodiments, the same substance can serve as both a flavoring agent and a taste masking agent.

As used herein, the term “coloring agent,” “colorant,” or “pigment” refers to a substance used to impart a color, e.g., to improve the appearance and attractiveness by the subject. Color consistency can be significant, as it allows easy identification of a medication to the subject. Furthermore, colors often improve the aesthetic look and feel of medications. By increasing these organoleptic properties, a subject is more likely to adhere to their schedule and therapeutic objectives will also have a better outcome for the subject.

As used herein, the term “release modifier” refers to a substance employed to modify the release of active ingredient from the oral dissolvable film and/or to modify the absorption of active ingredient when administered to the subject. The modified drug release can be contrasted to an immediate release (IR), and includes, e.g., an extended release (XR) or delayed release (DR).

As used herein, the term “adjuvant” refers to a substance (e.g., pharmacological or immunological agent) that modifies (e.g., increases) the effect or efficacy of the active ingredient.

As used herein, the term “sweetener” or “sweetening agent” refers to a substance that provides a sweet taste. The sweetener can be natural or artificial. Suitable sweeteners include sugars (e.g., glucose, corn syrup, fructose, and sucrose) as well as sugar substitutes (e.g., honey, honey granules, aspartame, neotame, acesulfame potassium (Ace-K), saccharin, sodium saccharine, advantame, sucralose, monk fruit extract (mogrosides), stevia, rebaudioside A, sorbitol, xylitol, and lactitol).

As used herein, the term “solubilizer & emulsifier” or “emulsifier” refers to a substance capable of forming or promoting an emulsion. In particular reference to the oral dissolvable films described herein, the emulsifier promotes the separation of phases (e.g., aqueous and lipids), while allowing them to be mixed. Suitable emulsifiers include, e.g., Polysorbate 80, glycerin, propylene glycol, and polyethylene glycol.

The term “emulsion” refers to a mixture of two or more liquids that are normally immiscible (unmixable or unblendable) owing to liquid-liquid phase separation. Two liquids can form different types of emulsions. As an example, oil and water can form, first, an oil-in-water emulsion, in which the oil is the dispersed phase, and water is the continuous phase. Second, they can form a water-in-oil emulsion, in which water is the dispersed phase and oil is the continuous phase. Multiple emulsions are also possible, including a “water-in-oil-in-water” emulsion and an “oil-in-water-in-oil” emulsion. Emulsions, being liquids, do not exhibit a static internal structure. The droplets dispersed in the continuous phase (sometimes referred to as the “dispersion medium”) are usually assumed to be statistically distributed to produce roughly spherical droplets. When molecules are ordered during liquid-liquid phase separation, they form liquid crystals rather than emulsions. Lipids, used by all living organisms, are one example of molecules able to form either emulsions (e.g., spherical micelles; Lipoproteins) or liquid crystals (lipid bilayer membranes).

The droplets may be amorphous, liquid-crystalline, or any mixture thereof. The diameters of the droplets constituting the dispersed phase usually range from approximately 10 nm to 100 μm; i.e., the droplets may exceed the usual size limits for colloidal particles. An emulsion is termed an oil/water (o/w) emulsion if the dispersed phase is an organic material and the continuous phase is water or an aqueous solution and is termed water/oil (w/o) if the dispersed phase is water or an aqueous solution and the continuous phase is an organic liquid (an “oil”).

Two special classes of emulsions—microemulsions and nanoemulsions, with droplet sizes below 100 nm—appear translucent. This property is due to the fact that light waves are scattered by the droplets only if their sizes exceed about one-quarter of the wavelength of the incident light. Since the visible spectrum of light is composed of wavelengths between 390 and 750 nanometers (nm), if the droplet sizes in the emulsion are below about 100 nm, the light can penetrate through the emulsion without being scattered. Due to their similarity in appearance, translucent nanoemulsions and microemulsions are frequently confused. Unlike translucent nanoemulsions, which require specialized equipment to be produced, microemulsions are spontaneously formed by “solubilizing” oil molecules with a mixture of surfactants, co-surfactants, and co-solvents. The required surfactant concentration in a microemulsion is, however, several times higher than that in a translucent nanoemulsion, and significantly exceeds the concentration of the dispersed phase. Because of many undesirable side-effects caused by surfactants, their presence is disadvantageous or prohibitive in many applications. In addition, the stability of a microemulsion is often easily compromised by dilution, by heating, or by changing pH levels.

The term “lipid” refers to a group of naturally occurring molecules that include fats, waxes, sterols, fat-soluble vitamins (such as vitamins A, D, E, and K), monoglycerides, diglycerides, triglycerides, phospholipids, and others. “Lipid” may also refer to ethoxylated fatty alcohols such as oleth-10 and laureth-10 and mixtures of ethoxylated mono and diglycerides such as PEG-16 macadamia glycerides and PEG-10 sunflower glycerides. The compounds are hydrophobic or amphiphilic small molecules. The amphiphilic nature of some lipids allows them to form structures such as vesicles, liposomes, or membranes in an aqueous environment. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits). Although the term lipid is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol. Suitable lipids include, e.g., almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, marula oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, Shea butter, soybean oil, sunflower oil, walnut oil, and watermelon seed oil.

As used herein, the term “fragrance” (alternatively known as an odorant or aroma compounds) refers to a substance employed to impart a desired smell or odor.

As used herein, the term “pH adjusting agent” refers to a substance that, when added to an aqueous solution (e.g., slurry), will change the pH. For example, the pH adjusting agent can be an acid, such that when added to an aqueous solution (e.g., slurry), it will decrease the pH. Alternatively, the pH adjusting agent can be a base, such that when added to an aqueous solution (e.g., slurry), it will increase the pH. The base can be an organic base (e.g., sodium bicarbonate) or inorganic base (e.g., sodium hydroxide), and the acid can be at least one of an inorganic acid (e.g., hydrochloric acid) and/or an organic acid (e.g., citric acid, malic acid, tartaric acid, etc.).

As used herein, the term “buffering agent” refers to a weak acid or weak base used to maintain the pH (e.g., acidity or basicity) of a solution (e.g., slurry) near a chosen value after the addition of another acid or base. That is, the function of a buffering agent is to prevent a rapid change in pH when acids or bases are added to the solution (e.g., slurry). Buffering agents have variable properties-some are more soluble than others; some are acidic while others are basic. The acid can me an organic acid, mineral acid, or combination thereof. Likewise, the base can me an organic base, inorganic base, or combination thereof.

The term “lubricant” or “glidant” refers to a substance added to the formulation (e.g., slurry) to improve processing characteristics. For example, the lubricant can enhance flow of the slurry by reducing interparticulate friction. Suitable lubricants include, e.g., magnesium stearate, calcium stearate, stearic acid, hydrogenated vegetable oil (e.g., Sterotex, Lubritab, and Cutina), mineral oil, polyethylene glycol 4000-6000 (PEG), sodium lauryl sulfate (SLS), sodium hyaluronate, sucrose esters, glyceryl behenate (stelliesters), dimethyl phthalate, diethyl phthalate, dibutyl phthalate, tributyl citrate, triethyl citrate, acetyl citrate, triacetin, dioctyl adipate, diethyl adipate, di(2-methylethyl) adipate, dihexyl adipate, partial fatty acid esters of sugars, polyethylene glycol fatty acid esters, polyethylene glycol fatty alcohol ethers, polyethylene glycol sorbitan fatty acid esters, 2-ethoxy ethanol, ethyl alcohol, propyl alcohol, butyl alcohol, pentyl alcohol, hexyl alcohol, heptyl alcohol, octyl alcohol, dibutyl tartrate, castor oil, or any combination thereof.

As used herein, the term “stabilizer” refers to a substance employed to that is used to prevent degradation of any one of more substances present in the slurry and/or oral dissolvable film. This would include the active ingredient as well as any of the inactive ingredients (e.g., excipients or additives).

As used herein, the term “antioxidant” refers to a substance that inhibits or prevents oxidation of any one of more substances present in the slurry and/or oral dissolvable film. This would include the active ingredient as well as any of the inactive ingredients (e.g., excipients or additives). Examples of antioxidants include, e.g., ascorbic acid (vitamin C), vitamin A, α-tocopherol (vitamin E), beta-carotene, glutathione, ubiquinol (coenzyme Q), and selenium.

As used herein, the term “anti-tacking agent” refers to a substance employed to prevent the formation of lumps (caking) of powdered or granulated materials. Use of the anti-tacking agent can result in the ease of flowability of the solid powders used to form the slurry. Crystalline solids often cake by formation of liquid bridge and subsequent fusion of microcrystals. Amorphous materials can cake by glass transitions and changes in viscosity. Polymorphic phase transitions can also induce caking. Examples include, e.g., calcium silicate, calcium carbonate, and magnesium carbonate.

As used herein, the term “humectant” refers to a substance used to keep the slurry and/or oral dissolvable film moist. A humectant attracts and retains the moisture in the air nearby via absorption, drawing the water vapor into or beneath the oral dissolvable film's surface. This is the opposite use of a hygroscopic material where it is used as a desiccant used to draw moisture away. Humectants can be used in oral dissolvable films to increase the solubility of active ingredients, increasing the active ingredients' ability to penetrate a mucosal surface, or its activity time. Examples include, e.g., propylene glycol, hexylene glycol, butylene glycol, aloe vera gel, alpha hydroxy acids (e.g., lactic acid), glyceryl triacetate, and sugar alcohols or polyols (e.g., glycerol, sorbitol, xylitol, and maltitol).

As used herein, the term “permeation enhancer” refers to a substance employed to increase the delivery the active ingredient, when administered in vivo (e.g., orally), across the desired body surface (e.g., oral mucosa, such as buccal, sublingual, mucosa, or gingival; or an intestinal surface), resulting in an increased absorption of the active ingredient.

As used herein, the term “preservative” refers to a substance that is added to prevent decomposition by microbial growth or by undesirable chemical changes. Some typical preservatives used in pharmaceutical formulations include: antioxidants like vitamin A, vitamin E, vitamin C, vitamin C palmitate, retinyl palmitate, and selenium; the amino acids cysteine and methionine; citric acid and sodium citrate; synthetic preservatives like the parabens: methyl paraben and propyl paraben. With the oral dissolvable films described herein, the preservative can include, e.g., any one or more of sodium benzoate, benzoic acid, sodium nitrite, sodium sorbate, potassium sorbate, and ascorbic acid.

As used herein, the term “oil” any nonpolar chemical substance that is a viscous liquid at ambient temperatures and is both hydrophobic (does not mix with water, literally “water fearing”) and lipophilic (mixes with other oils, literally “fat loving”). Oils have a high carbon and hydrogen content and are usually flammable and surface active. Most oils are unsaturated lipids that are liquid at room temperature. The general definition of oil includes classes of chemical compounds that may be otherwise unrelated in structure, properties, and uses. Oils may be animal or vegetable in origin, and may be volatile or non-volatile. They are typically used for food (e.g., olive oil).

As used herein, the term “oil carrier” refers to an oil, as described herein, useful as a solvent.

As used herein, the term “aqueous liquid” refers to a liquid that includes water.

As used herein, the term “hot air oven” refers to an oven that emits convection heat.

The term “convection heat” refers to heat obtained by convection. “Convection” refers to the transfer of heat from one place to another by the movement of fluids (e.g., gas, such as air). Convection is usually the dominant form of heat transfer in liquids and gases. Although often discussed as a distinct method of heat transfer, convective heat transfer involves the combined processes of unknown conduction (heat diffusion) and advection (heat transfer by bulk fluid flow). Two types of convective heat transfer can be distinguished: (1) free or natural convection and (2) forced convection. The convection heat employed in the methods of the present invention can include (1) free or natural convection and/or (2) forced convection.

Free or natural convection occurs when fluid motion is caused by buoyancy forces that result from the density variations due to variations of thermal temperature in the fluid. In the absence of an internal source, when the fluid is in contact with a hot surface, its molecules separate and scatter, causing the fluid to be less dense. As a consequence, the fluid is displaced while the cooler fluid gets denser and the fluid sinks. Thus, the hotter volume transfers heat towards the cooler volume of that fluid. Familiar examples are the upward flow of air due to a fire or hot object and the circulation of water in a pot that is heated from below. In contrast, forced convection occurs when a fluid is forced to flow over the surface by an internal source such as fans, by stirring, and pumps, or creating an artificially induced convection current.

The term “thickness” refers to the distance between opposite sides of the oral dissolvable film. The thickness is the smallest of the three dimensions (length, width, and thickness). The thickness of the film can be measured by a micrometer screw gauge or calibrated digital Vernier Calipers. The thickness can be evaluated at five different locations (four corners and one at center) and in specific embodiments may be significant to ascertain uniformity in the thickness of the film, as this may be directly related to accuracy of dose distribution in the film.

The term “mass” refers to a measurement of how much matter is in an object. Mass is a combination of the total number of atoms, the density of the atoms, and the type of atoms in an object. Mass is usually measured in grams (which is abbreviated as g) or milligrams (which is abbreviated as mg).

The term “drug load” or “load of active ingredient” refers to the amount of active pharmaceutical ingredient present in the oral dissolvable film (or slurry). For example, in specific embodiments the oral dissolvable film can have a high drug load, such that the active pharmaceutical ingredient is present therein in a relatively high amount (e.g., above 30 wt. % of the oral dissolvable film).

The term “density” refers to the mass per unit volume of an object (e.g., oral dissolvable film). Density is calculated by dividing the mass of an object by the volume of the object. The volume of an object can be stated as cubic centimeters or milliliters as both are equivalent.

The term “loss on drying (LOD)” refers to the loss of weight expressed as percentage w/w resulting from water and/or volatile matter that can be driven off under specified conditions from an object (e.g., oral dissolvable film). In this technique, a sample of material (e.g., oral dissolvable film) is weighed, heated in an oven for an appropriate period, cooled in the dry atmosphere of a desiccator, and then reweighed. The difference in weight is the loss on drying (LOD). For example, the oral dissolvable film can have a loss on drying (LOD) of 10±2 wt. %.

The term “tack” refers to the tenacity with which the oral dissolvable film adheres to an accessory (a piece of paper) that has been pressed into contact with the film.

The term “tensile strength” refers to the maximum stress applied to a point at which the oral dissolvable film specimen breaks. It is calculated by the applied load at rupture divided by the cross-sectional area of oral dissolvable film, as given in the equation below:

Tensile strength=Load at failure×100/Film thickness×Film width

The term “percent elongation” refers to the relative increase in amount in length upon application of stress. When stress is applied on a film sample, it gets stretched. This is referred to as strain. Strain is basically the deformation of film before it gets broken due to stress. It can be measured by using hounsfield universal testing machine. Generally, elongation of the film increases as the plasticizer content increases. It is calculated by the formula:

% Elongation=Increase in length of film×100/Initial length of film

The term “tear resistance” refers to the resistance which a film offers when some load or force is applied on the film specimen. Specifically, it is the maximum force required to tear the specimen. The load mainly applied can be of a very low rate (e.g., 51 mm/min). The unit of tear resistance is Newton or pounds-force.

The term “Young's modulus” or “elastic modulus” refers to the measure of stiffness of a dissolvable film. It is represented as the ratio of applied stress over strain in the region of elastic deformation as follows:

Young's modulus=Slope×100/Film thickness×Cross head speed

Hard and brittle strips demonstrate a high tensile strength and Young's modulus with small elongation.

The term “folding endurance” refers to number of times the film can be folded without breaking or without any visible crack. Folding endurance gives the brittleness of a film. The method followed to determine endurance value is that the film specimen is repeatedly folded at the same place until it breaks, or a visible crack is observed. The number of times the film is folded without breaking or without any visible crack is the calculated folding endurance value.

The term “drug content uniformity,” “uniformity of dosage unit” or “CU” refers to the degree of uniformity in the amount of drug substance among dosage units, and unless otherwise specified, is set forth in USP-NF General Chapter <905> Uniformity of Dosage Units.

The manufacture of orally dissolving films can be carried out by various methods such as: (1) casting (e.g., solvent casting or semi-solid casting), (2) extrusion (e.g., hot melt extrusion or solid dispersion), and (3) rolling. These methods of manufacturing oral dissolvable films are generally well-known to the skilled artisans. See, e.g., “Manufacturing Techniques of Orally Dissolving Films,” Pharmaceutical Technology, Volume 35, Issue 1 (Jan. 2, 2011); “Current Advances in Drug Delivery Through Fast Dissolving/Disintegrating Dosage Forms,” Vikas Anand Saharan, pp. 318-356 (39) (2017); A short review on “A novel approach in oral fast dissolving drug delivery system and their patents,” M. N. Siddiqui, G. Garg, P. K. Sharma, Adv. Biol. Res., 5 (2011), pp. 291-303; “Orally disintegrating films: A modern expansion in drug delivery system,” Ifran et al., Saudi Pharmaceutical Journal, Volume 24, Issue 5, pp. 537-546 (September 2016). “Development and characterization of pharmacokinetic parameters of fast-dissolving films containing levocetirizine,” D. R. Choudhary, V. A. Patel, U.K. Chhalotiya, H. V. Patel, A. J. Kundawala; Sci. Pharm., 80 (2012), pp. 779-787; “Orally disintegrating preparations: recent advancement in formulation and technology,” R. R. Thakur, D. S. Rathore, S. Narwal; J. Drug Deliv. Therap., 2 (3) (2012), pp. 87-96; “Development of innovative orally fast disintegrating film dosage forms: a review,” B. P. Panda, N. S. Dey, M. E. B. Rao; Int. J. Pharm. Sci. Nanotechnol., 5 (2012), pp. 1666-1674.

Across multiple embodiments, substances present in the orally dissolvable film are characterized by the amount of substance present therein. The substance can be the active pharmaceutical ingredient(s) and/or any one or more of the excipients. Unless expressly stated otherwise, the amount of substance present therein is based on an anhydrous film (e.g., an orally dissolvable film containing no water). A notable exception is the amount of water (moisture) present in the dissolvable film. By way of illustration, reference is made to the product illustrated in the table below (amounts calculated for 100 strips batch.). A dissolvable film can be prepared from a slurry, in which an active ingredient (CBD isolate) is present in 50 mg per 230.82 mg strip (21.66 wt. %). This can be calculated as follows:

$$\frac{mass active ingedient ( mg )}{mass dry weight film ( mg )} \times 100 = \frac{50 mg}{230.82 mg} \times 100 = 0.2166 = 21.66 wt . \%$$

In order to arrive at this calculation, the water is not included in the mass of the dry weight (anhydrous) strip. This is contrasted with the slurry, in which active ingredient (CBD isolate) is present in 6.5 wt. %. In arriving at this calculation, the water is included in the mass of the slurry. This can be calculated as follows:

$$\frac{mass active ingedient ( mg )}{mass of slurry ( mg )} \times 100 = \frac{50 mg}{769.4 mg} \times 100 = 0.065 = 6.5 wt . \%$$

The mass of the slurry (769.4 mg) is obtained from the amount (mass) of the dry weight (anhydrous) strip (230.82 mg) plus the amount (mass) of the purified water added to the slurry (538.58 mg).

| | | Amount | | Amount | |
|---|---|---|---|---|---|
| | | (mg)/ | % W/W | (g)/ | % W/W |
| Material | Function | Strip | Dry | 100 Strip | Slurry |
| CBD Isolate | Active | 50 | 21.66 | 5.00 | 6.50 |
| | ingredient | | | | |
| Tween 20 | Hydrophilic | 25 | 10.83 | 2.50 | 3.25 |
| | Surfactant | | | | |
| Span 80 | Lipophilic | 5 | 2.17 | 0.50 | 0.65 |
| | Surfactant | | | | |
| Propylene | Lipophilic | 50 | 21.66 | 5.00 | 6.50 |
| Glycol | Surfactant/ | | | | |
| Monocaprylate | Solvent for | | | | |
| | API | | | | |

Flavors

| | | | | | |
|---|---|---|---|---|---|
| Sucralose | Sweetener | 1.573 | 0.68 | 0.16 | 0.20 |
| USP/NF | | | | | |
| Mint Flavor | Flavor | 7.289 | 3.16 | 0.73 | 0.95 |

Film Forming System

| | | | | | |
|---|---|---|---|---|---|
| Modified Food | Film Former | 56.23 | 24.36 | 5.62 | 7.31 |
| Starch | Polymer | | | | |
| Pullulan | Film Former | 20.03 | 8.68 | 2.00 | 2.60 |
| | Polymer | | | | |
| Glycerin | Plasticizer | 15.55 | 6.74 | 1.56 | 2.02 |
| 99.7% USP | | | | | |
| Potassium | Antimicrobial | 0.1 | 0.04 | 0.01 | 0.01 |
| Sorbate | | | | | |
| Yellow 5 | Coloring | 0.03 | 0.01 | 0.00 | 0.00 |
| | Agent | | | | |
| Red 40 | Coloring | 0.015 | 0.01 | 0.00 | 0.00 |
| | Agent | | | | |
| Purified | N/A | 538.58 | | 53.86 | 70.00 |
| Water* | | | | | |
| | TOTAL | 230.82 | 100 | 76.94 | 100.00 |

*Purified Water amount can be adjusted as per need. It is not part of dry weight strip formulation.

As used herein, the term “glyceryl monocaprylate” refers to the substance having the IUPAC name 1,3-dihydroxypropan-2-yl octanoate; CAS Number 4228-48-2; chemical formula C₁₁H₂₂O₄; and molar mass 218.29 g·mol-1. When present in an oral dissolvable film described herein, the glyceryl monocaprylate can function at least as a lipophilic or hydrophobic surfactant.

As used herein, the term “propylene glycol monocaprylate” refers to the substance having the IUPAC name 2-hydroxypropyl octanoate; CAS Number 23794-30-1, 68332-79-6; chemical formula C₁₁H₂₂O₃; and molar mass 202.29 g·mol-1. When present in an oral dissolvable film described herein, the propylene glycol monocaprylate can function at least as a lipophilic or hydrophobic surfactant.

As used herein, the term “glyceryl monooleate” refers to the substance having the IUPAC name 2,3-dihydroxypropyl (Z)-octadec-9-enoate; CAS Number 111-03-5, 25496-72-4, 67701-32-0, 37220-82-9; chemical formula C₂₁H₄₀O₄; and molar mass 356.5 g·mol-1. When present in an oral dissolvable film described herein, the glyceryl monooleate can function at least as a lipophilic or hydrophobic surfactant.

As used herein, the term “propylene glycol monolaurate” refers to the substance having the IUPAC name 2-hydroxypropyl dodecanoate; CAS Number 142-55-2, 27194-74-7; chemical formula C₁₅H₃₀O₃; and molar mass 258.4 g·mol-1. When present in an oral dissolvable film described herein, the propylene glycol monolaurate can function at least as a lipophilic or hydrophobic surfactant.

As used herein, the term “glyceryl caprylate/caprate” refers to the substance having the IUPAC name 11-(2,3-dihydroxypropoxycarbonyl)heptadecanoate; CAS Number 73398-61-5; chemical formula C₁₅H₃₀O₃; and molar mass 387.5 g·mol-1. When present in an oral dissolvable film described herein, the glyceryl caprylate/caprate can function at least as a lipophilic or hydrophobic surfactant.

As used herein, the term “glyceryl monolinoleate” refers to the substance having the IUPAC name 2,3-dihydroxypropyl (9E,12E)-octadeca-9,12-dienoate; CAS Number 2277-28-3; chemical formula C₂₁H₃₈O₄; and molar mass 354.52 g·mol-1. When present in an oral dissolvable film described herein, the glyceryl monolinoleate can function at least as a lipophilic or hydrophobic surfactant.

As used herein, the term “sorbitan monooleate (Span 80)” refers to the substance having the IUPAC name [(2R)-2-[(2R,3R,4S)-3,4-dihydroxyoxolan-2-yl]-2-hydroxyethyl] (Z)-octadec-9-enoate; CAS Number 1338-43-8, 9015-08-1; chemical formula C₂₄H₄₄O₆; and molar mass 428.6 g·mol-1. When present in an oral dissolvable film described herein, the sorbitan monooleate (Span 80) can function at least as a lipophilic or hydrophobic surfactant.

As used herein, the term “glyceryl dibehenate” refers to the substance having the IUPAC name docosanoic acid; propane-1,2,3-triol; CAS Number 99880-64-5; chemical formula C₂₅H₅₂O₅; and molar mass 432.7 g·mol-1. When present in an oral dissolvable film described herein, the glyceryl dibehenate can function at least as a lipophilic or hydrophobic surfactant.

As used herein, the term “propylene glycol dilaurate” refers to the substance having the IUPAC name 2-dodecanoyloxypropyl dodecanoate; CAS Number 22788-19-8; chemical formula C₂₇H₅₂O₄; and molar mass 440.7 g·mol-1. When present in an oral dissolvable film described herein, the propylene glycol dilaurate can function at least as a lipophilic or hydrophobic surfactant.

As used herein, the term “glyceryl tricaprylate/tricaprate” refers to the substance having the IUPAC name 2,3-di(octanoyloxy)propyl octanoate; CAS Number 538-23-8; chemical formula C₂₇H₅₀O₆; and molar mass 470.7 g·mol-1. When present in an oral dissolvable film described herein, the glyceryl tricaprylate/tricaprate can function at least as a lipophilic or hydrophobic surfactant.

As used herein, the term “glycerol tricaprylate/caprate” refers to the substance having the IUPAC name 11-(2,3-dihydroxypropoxycarbonyl)heptadecanoate; CAS Number 73398-61-5; chemical formula C₂₁H₃₉O₆; and molar mass 387.5 g·mol-1. When present in an oral dissolvable film described herein, the glycerol tricaprylate/caprate can function at least as a lipophilic or hydrophobic surfactant.

As used herein, the term “decaglycerol mono oleate” refers to the substance having the IUPAC name (Z)-octadec-9-enoic acid; propane-1,2,3-triol; CAS Number; chemical formula C₄₈H₁₁₄O₃₂; and molar mass 1203.4 g·mol-1. When present in an oral dissolvable film described herein, the decaglycerol mono oleate can function at least as a lipophilic or hydrophobic surfactant.

As used herein, the term “decaglycerol di oleate” refers to the substance having the IUPAC name [2-hydroxy-3-[2-hydroxy-3-[2-hydroxy-3-[2-hydroxy-3-[2-hydroxy-3-[2-hydroxy-3-[2-hydroxy-3-[2-hydroxy-3-[2-hydroxy-3-[2-hydroxy-3-[(Z)-octadec-9-enoyl]oxypropoxy]propoxy]propoxy]propoxy]propoxy]propoxy]propoxy]propoxy]propoxy]prop yl] (Z)-octadec-9-enoate; CAS Number 33940-99-7; chemical formula C₆₆H₁₂₆O₂₃; and molar mass 1287.7 g·mol-1. When present in an oral dissolvable film described herein, the decaglycerol di oleate can function at least as a lipophilic or hydrophobic surfactant.

As used herein, the term “oleoyl macrogolglycerides” refers to ingredients, obtained from apricot kernel oils. Oleoyl macrogolglycerides include complex mixtures, constituted of mono-(MG), di-(DG) and triglycerides (TG) and mono-(MPEGE) and di PEG-6 esters (DPEGE) of oleic acid (18:1). When present in an oral dissolvable film described herein, the oleoyl macrogolglycerides can function at least as a lipophilic or hydrophobic surfactant.

As used herein, the term “lauroyl macrogolglycerides” refers to ingredients, obtained from corn oils. Lauroyl macrogolglycerides include complex mixtures, constituted of mono-(MG), di-(DG) and triglycerides (TG) and mono-(MPEGE) and di PEG-6 esters (DPEGE) of linoleic acid (18:2). When present in an oral dissolvable film described herein, the lauroyl macrogolglycerides can function at least as a lipophilic or hydrophobic surfactant.

As used herein, the term “stearoyl macrogolglycerides” refers to a mixture of monoesters, diesters, and triesters of glycerol and monoesters and diesters of polyethylene glycols. The polyethylene glycols used have a mean molecular weight between 300 and 4000. They are produced by partial alcoholysis of saturated oils, mainly containing triglycerides of stearic acid, with polyethylene glycol, by esterification of glycerol and polyethylene glycol with fatty acids, or as mixtures of glycerol esters and ethylene oxide condensates with the fatty acids of the hydrogenated oils. The hydroxyl value is not less than 85 percent and not more than 115 percent of the labeled nominal value, and the saponification value is not less than 90 percent and not more than 110 percent of the labeled nominal value. Stearoyl macrogolglycerides may contain free polyethylene glycols. When present in an oral dissolvable film described herein, the stearoyl macrogolglycerides can function at least as a lipophilic or hydrophobic surfactant.

As used herein, the term “stearoyl polyoxylglycerides” refers to a mixture of monoesters, diesters, and triesters of glycerol and monoesters and diesters of polyethylene glycols. The polyethylene glycols used have a mean molecular weight between 300 and 4000. They are produced by partial alcoholysis of saturated oils, mainly containing triglycerides of stearic acid, with polyethylene glycol, by esterification of glycerol and polyethylene glycol with fatty acids, or as mixtures of glycerol esters and ethylene oxide condensates with the fatty acids of the hydrogenated oils. The hydroxyl value is not less than 85 percent and not more than 115 percent of the labeled nominal value, and the saponification value is not less than 90 percent and not more than 110 percent of the labeled nominal value. Stearoyl polyoxylglycerides may contain free polyethylene glycols. When present in an oral dissolvable film described herein, the stearoyl polyoxylglycerides can function at least as a lipophilic or hydrophobic surfactant.

As used herein, the term “polyoxyethylene” refers to the substance having the IUPAC name 1-(2-methoxyethoxy)hexadecane; CAS Number; chemical formula C₁₉H₄₀O₂; and molar mass 300.5 g·mol-1. When present in an oral dissolvable film described herein, the polyoxyethylene can function at least as a lipophilic or hydrophobic surfactant.

As used herein, the term “caprylic/capric glycerides” refers to an oily liquid made from palm kernel or coconut oil. Caprylic/capric glycerides includes a mixed ester composed of caprylic and capric fatty acids attached to a glycerin backbone. Caprylic/capric glycerides are made up mostly of triglycerides whose fatty acids are chains ranging from 6-12 carbon atoms, in this case the ester is comprised of capric (10 carbon atoms) and caprylic (8 carbon atoms). Caprylic/capric glycerides are naturally occurring in coconut and palm kernel oils at lower levels. Caprylic/capric glycerides can also be obtained when the oils are split and the specific fatty acid (capric acid and caprylic acid are isolated and recombined with the glycerin backbone to form the pure caprylic/capric glycerides which are then further purified (bleached and deodorized) using clay, heat and steam. When present in an oral dissolvable film described herein, the caprylic/capric glycerides can function at least as a lipophilic or hydrophobic surfactant.

As used herein, the term “poloxamers” refers to block copolymers of poly(ethylene oxide) (PEO) and poly(propylene oxide) (PPO), which have an amphiphilic character and useful association and adsorption properties emanating from this. Poloxamers find use in many applications that require solubilization or stabilization of compounds and also have notable physiological properties, including low toxicity. When present in an oral dissolvable film described herein, the poloxamer can function at least as a lipophobic or hydrophilic surfactant.

As used herein, the term “polyoxyl castor oil” refers to a mixture of triricinoleate esters of ethoxylated glycerol with small amounts of polyethylene glycol (macrogol) ricinoleate and the corresponding free glycols. When present in an oral dissolvable film described herein, the polyoxyl castor oil can function at least as a lipophobic or hydrophilic surfactant.

As used herein, the term “polyethylene-polypropylene glycol” refers to a nonionic polyoxyethylene-polyoxypropylene copolymers used primarily as emulsifying or solubilizing agents. The polyoxyethylene segment is hydrophilic while the polyoxypropylene segment is hydrophobic. The polyethylene-polypropylene glycol is chemically similar in composition, differing only in the relative amounts of propylene and ethylene oxides added during manufacture. Their physical and surface-active properties vary over a wide range. When present in an oral dissolvable film described herein, the polyethylene-polypropylene glycol can function at least as a lipophobic or hydrophilic surfactant.

As used herein, the term “polyoxyethylene sorbitan monolaurate (Tween 20)” refers to a polysorbate-type nonionic surfactant formed by the ethoxylation of sorbitan before the addition of lauric acid. Its stability and relative nontoxicity allow it to be used as a detergent and emulsifier in a number of scientific applications. When present in an oral dissolvable film described herein, the polyoxyethylene sorbitan monolaurate (Tween 20) can function at least as a lipophobic or hydrophilic surfactant.

As used herein, the term “Tween 80” refers to a polysorbate-type nonionic surfactant formed by the ethoxylation of sorbitan before the addition of lauric acid. Its stability and relative nontoxicity allow it to be used as a detergent and emulsifier in a number of scientific applications. When present in an oral dissolvable film described herein, the Tween 80 can function at least as a lipophobic or hydrophilic surfactant.

As used herein, the term “polyoxyethylenesorbitan monostearate (Tween 60)” refers to a polysorbate-type nonionic surfactant formed by the ethoxylation of sorbitan before the addition of lauric acid. Its stability and relative nontoxicity allow it to be used as a detergent and emulsifier in a number of scientific applications. When present in an oral dissolvable film described herein, the polyoxyethylenesorbitan monostearate (Tween 60) can function at least as a lipophobic or hydrophilic surfactant.

As used herein, the term “decyl glucoside” refers to the substance having the IUPAC name (3R,4S,5S,6R)-2-decoxy-6-(hydroxymethyl)oxane-3,4,5-triol; CAS Number 54549-25-6, 68515-73-1; chemical formula C₁₆H₃₂O₆; and molar mass 320.42 g·mol-1. When present in an oral dissolvable film described herein, the decyl glucoside can function at least as a lipophobic or hydrophilic surfactant.

As used herein, the term “lauryl glucoside” refers to the substance having the IUPAC name (2R,3R,4S,5S,6R)-2-dodecoxy-6-(hydroxymethyl)oxane-3,4,5-triol; CAS Number 59122-55-3; chemical formula C₁₈H₃₆O₆; and molar mass 348.5 g·mol-1. When present in an oral dissolvable film described herein, the lauryl glucoside can function at least as a lipophobic or hydrophilic surfactant.

As used herein, the term “octyl glucoside” refers to the substance having the IUPAC name 2-(hydroxymethyl)-6-octoxyoxane-3,4,5-triol; CAS Number 4742-80-7; chemical formula C₁₄H₂₈O₆; and molar mass 292.37 g·mol-1. When present in an oral dissolvable film described herein, the octyl glucoside can function at least as a lipophobic or hydrophilic surfactant.

As used herein, the term “Triton X-100” refers to a nonionic surfactant that has a hydrophilic polyethylene oxide chain (on average it has 9.5 ethylene oxide units) and an aromatic hydrocarbon lipophilic or hydrophobic group. The hydrocarbon group is a 4-(1,1,3,3-tetramethylbutyl)-phenyl group. The substance has the IUPAC name 2-[4-(2,4,4-trimethylpentan-2-yl)phenoxy]ethanol; CAS Number 2315-67-5, 63869-93-2, 9002-93-1; chemical formula C₁₆H₂₆O₂; and molar mass 250.38 g·mol-1. When present in an oral dissolvable film described herein, the Triton X-100 can function at least as a lipophobic or hydrophilic surfactant.

As used herein, the term “nonoxynol 9” (sometimes abbreviated N-9) refers to the substance having the IUPAC name 2-[2-[2-[2-[2-[2-[2-[2-[2-(4-nonylphenoxy)ethoxy]ethoxy]ethoxy]ethoxy]ethoxy]ethoxy]ethoxy]ethoxy]ethanol; CAS Number 26571-11-9, 26027-38-3, 14409-72-4; chemical formula C₃₃H₆₀O₁₀; and molar mass 616.8 g·mol-1. When present in an oral dissolvable film described herein, the nonoxynol 9 can function at least as a lipophobic or hydrophilic surfactant.

As used herein, the term “sodium lauryl sulfate” refers to the substance having the IUPAC name sodium dodecyl sulfate; CAS Number 151-21-3, 1335-72-4, 8012-56-4; chemical formula NaSO₄C₁₂H₂₅ or C₁₂H₂₅O₄S·Na or C₁₂H₂₅NaO₄S; and molar mass 288.38 g·mol-1. When present in an oral dissolvable film described herein, the sodium lauryl sulfate can function at least as a lipophobic or hydrophilic surfactant.

As used herein, the term “potassium lauryl sulfate” refers to the substance having the IUPAC name potassium dodecyl sulfate; CAS Number 4706-78-9; chemical formula C₁₂H₂₅KO₄S; and molar mass 304.49 g·mol-1. When present in an oral dissolvable film described herein, the potassium lauryl sulfate can function at least as a lipophobic or hydrophilic surfactant.

As used herein, the term “Brij” or “Brij®” refers to a family of non-ionic surfactants. Suitable Brij surfactants include, e.g., Brij 78 (C₁₈H₃₇E₂₀), Brij 98 (C₁₈H₃₅E₂₀) and Brij 700 (C₁₈H₃₇E₁₀₀) (where E represents the OCH₂CH₂ unit of the poly(ethylene oxide) chain) at 25, 37 and 40° C. Additional Brij surfactants include, e.g., Brij 23, Brij 30, Brij 35, Brij® S20, Brij® O20, Brij® O10, Brij® C10, Brij® C20, Brij® L4, Brij® S2, Brij® S20 and other Brij® products. Specific polyoxyethylene alkyl ethers include Brij® L4 and Brij® S20. The Brij® products are commercially available from Sigma-Aldrich (St. Louis, Mo.) and Croda (East Yorkshire, U.K.). When present in an oral dissolvable film described herein, the Brij can function at least as a lipophobic or hydrophilic surfactant.

As used herein, the term “glyceryl laurate” refers to the substance having the IUPAC name 1,3-diacetyloxypropan-2-yl undecanoate; CAS Number 120602-37-1; chemical formula C₁₈H₃₂O₆; and molar mass 344.4 g·mol-1. When present in an oral dissolvable film described herein, the glyceryl laurate can function at least as a lipophobic or hydrophilic surfactant.

As used herein, the term “phospholipid” refers to the substance having the IUPAC [2-[decyl(hydroxy)phosphoryl]oxy-3-(10-methoxy-10-oxodecoxy)propyl] 2-(trimethylazaniumyl)ethyl phosphate; CAS Number; chemical formula C₂₉H₆₁NO₁₀P₂; and molar mass 645.7 g·mol-1 or derivatives thereof. When present in an oral dissolvable film described herein, the phospholipid can function at least as a lipophobic or hydrophilic surfactant.

As used herein, the term “n-dodecyl phosphocholine” refers to the substance having the IUPAC name dodecyl 2-(trimethylazaniumyl)ethyl phosphate; CAS Number 29557-51-5; chemical formula C₁₇H₃₈NO₄P; and molar mass 351.5 g·mol-1. When present in an oral dissolvable film described herein, the n-dodecyl phosphocholine can function at least as a lipophobic or hydrophilic surfactant.

As used herein, the term “cholesteryl ester” refers to the substance such as 17:1 cholesteryl ester, having the IUPAC name [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] (Z)-heptadec-9-enoate; CAS Number; chemical formula C₄₄H₇₆O₂; and molar mass 637.1 g·mol-1 or derivatives thereof including but not limited to, 17:0, 15:0, 22:4, 20:3, and 22:3 cholesteryl esters. When present in an oral dissolvable film described herein, the cholesteryl ester can function at least as a lipophobic or hydrophilic surfactant.

As used herein, the term “medium chain triglycerides oil” refers to the substance having a triglyceride with two to three fatty acids having an aliphatic tail of 6-12 carbon atoms. Medium chain triglyceride oils include, but are not limited to fatty acids such as, hexanoic or caproic acid, octanoic or caprylic acid, decanoic or capric acid, dodecanoic or lauric acid. When present in an oral dissolvable film described herein, the medium chain triglycerides oil can function at least as an oil carrier or as a lipophilic or hydrophobic solvent for the active ingredient.

As used herein, the term “coconut oil” refers to the substance having the IUPAC name (1-decanoyloxy-3-octanoyloxypropan-2-yl) dodecanoate; CAS Number 68991-68-4; chemical formula C₃₃H₆₂O₆; and molar mass 554.8 g·mol-1. When present in an oral dissolvable film described herein, the coconut oil can function at least as an oil carrier or as a lipophilic or hydrophobic solvent for the active ingredient.

As used herein, the term “corn oil” refers to the substance extracted from the germ of corn and its physically modified derivatives. Corn oil includes, but is not limited to, the glycerides of the fatty acids linoleic, oleic, palmitic and stearic acid, having the IUPAC name, CAS Number 8001-30-7; chemical formula; and molar mass g·mol-1. When present in an oral dissolvable film described herein, the corn oil can function at least as an oil carrier or as a lipophilic or hydrophobic solvent for the active ingredient.

As used herein, the term “olive oil” refers to the substance having the IUPAC name hexadecanoic acid;(9Z,12Z)-octadeca-9,12-dienoic acid;octadecanoic acid;(9Z,12Z,15Z)-octadeca-9,12,15-trienoic acid;(Z)-octadec-9-enoic acid; CAS Number 92044-96-7; chemical formula C₈₈H₁₆₄O₁₀; and molar mass 1382.2 g·mol-1. When present in an oral dissolvable film described herein, the olive oil can function at least as an oil carrier or as a lipophilic or hydrophobic solvent for the active ingredient.

As used herein, the term “palm oil” refers to the substance having the IUPAC name 1-hydroxypropan-2-olate;3-oxododecanoic acid; CAS Number 91052-70-9; chemical formula C₁₅H₂₉O₅; and molar mass 289.39 g·mol-1. When present in an oral dissolvable film described herein, the palm oil can function at least as an oil carrier or as a lipophilic or hydrophobic solvent for the active ingredient.

As used herein, the term “canola oil” refers to the substance derived from a variety of rapeseed that is low in erucic acid. Canola oil includes the oil produced from the seed of any of several cultivars of the plant family Brassicaceae. For example, canola oil includes oil extracted from seeds of the genus Brassica (Brassica napus, Brassica rapa, or Brassica juncea) from which the oil shall contain less than 2% erucic acid in its fatty acid profile and the solid component shall contain less than 30 micromoles of any one or any mixture of 3-butenyl glucosinolate, 4-pentenyl glucosinolate, 2-hydroxy-3 butenyl glucosinolate, and 2-hydroxy-4-pentenyl glucosinolate per gram of air-dry, oil-free solid. When present in an oral dissolvable film described herein, the canola oil can function at least as an oil carrier or as a lipophilic or hydrophobic solvent for the active ingredient.

As used herein, the term “safflower oil” refers to the substance extracted from the seeds of the safflower plant. When present in an oral dissolvable film described herein, the safflower oil can function at least as an oil carrier or as a lipophilic or hydrophobic solvent for the active ingredient.

As used herein, the term “sesame oil” refers to the substance extracted from sesame seeds. When present in an oral dissolvable film described herein, the sesame oil can function at least as an oil carrier or as a lipophilic or hydrophobic solvent for the active ingredient.

As used herein, the term “propylene glycol monocaprylate” refers to the substance having the IUPAC name 2-hydroxypropyl octanoate; CAS Number 23794-30-1, 68332-79-6; chemical formula C₁₁H₂₂O₃; and molar mass 202.29 g·mol-1. When present in an oral dissolvable film described herein, the propylene glycol monocaprylate can function at least as an oil carrier or as a lipophilic or hydrophobic solvent for the active ingredient.

As used herein, the term “propylene glycol monolaurate” refers to the substance having the IUPAC name 2-hydroxypropyl dodecanoate; CAS Number 142-55-2, 27194-74-7; chemical formula C₁₅H₃₀O₃; and molar mass 258.4 g·mol-1. When present in an oral dissolvable film described herein, the propylene glycol monolaurate can function at least as an oil carrier or as a lipophilic or hydrophobic solvent for the active ingredient.

As used herein, the term “glyceryl monolinoleate” refers to the substance having the IUPAC name 2,3-dihydroxypropyl (9E,12E)-octadeca-9,12-dienoate; CAS Number 2277-28-3; chemical formula C₂₁H₃₈O₄; and molar mass 354.5 g·mol-1. When present in an oral dissolvable film described herein, the glyceryl monolinoleate can function at least as an oil carrier or as a lipophilic or hydrophobic solvent for the active ingredient.

As used herein, the term “cetyl alcohol” refers to the substance having the IUPAC name hexadecan-1-ol; CAS Number 2277-28-3; chemical formula C₁₆H₃₄O; and molar mass 242.44 g·mol-1. When present in an oral dissolvable film described herein, the cetyl alcohol can function at least as an oil carrier or as a lipophilic or hydrophobic solvent for the active ingredient.

As used herein, the term “stearyl alcohol” refers to the substance having the IUPAC name octadecan-1-ol; CAS Number 112-92-5, 68911-61-5; chemical formula C₁₈H₃₈O; and molar mass 270.5 g·mol-1. When present in an oral dissolvable film described herein, the stearyl alcohol can function at least as an oil carrier or as a lipophilic or hydrophobic solvent for the active ingredient.

As used herein, the term “cetostearyl alcohol” refers to the substance having the IUPAC name hexadecan-1-ol;octadecan-1-ol; CAS Number 67762-27-0; chemical formula C₃₄H₇₂O₂; and molar mass 512.9 g·mol-1. When present in an oral dissolvable film described herein, the cetostearyl alcohol can function at least as an oil carrier or as a lipophilic or hydrophobic solvent for the active ingredient.

As used herein, the term “oleyl alcohol” refers to the substance having the IUPAC name (Z)-octadec-9-en-1-ol; CAS Number 143-28-2; chemical formula C₁₈H₃₆O; and molar mass 268.5 g·mol-1. When present in an oral dissolvable film described herein, the oleyl alcohol can function at least as an oil carrier or as a lipophilic or hydrophobic solvent for the active ingredient.

As used herein, the term “cyclosporine” refers to the substance having the IUPAC name 30-ethyl-33-[(E)-1-hydroxy-2-methylhex-4-enyl]-1,4,7,10,12,15,19,25,28-nonamethyl-6,9,18,24-tetrakis(2-methylpropyl)-3,21-di(propan-2-yl)-1,4,7,10,13,16,19,22,25,28,31-undecazacyclotritriacontane-2,5,8,11,14,17,20,23,26,29,32-undecone; CAS Number 59865-13-3; chemical formula C₆₂H₁₁₁N₁₁O₁₂; and molar mass 1202.6 g·mol-1. When present in an oral dissolvable film described herein, the cyclosporine can function at least as an active pharmaceutical ingredient.

As used herein, the term “ritonavir” refers to the substance having the IUPAC name 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate; CAS Number 155213-67-5; chemical formula C₃₇H₄₈N₆O₅S₂; and molar mass 875.106 g·mol-1. When present in an oral dissolvable film described herein, the ritonavir can function at least as an active pharmaceutical ingredient.

As used herein, the term “saquinavir” refers to the substance having the IUPAC name (2S)—N-[(2S,3R)-4-[(3S,4aS,8aS)-3-(tert-butylcarbamoyl)-3,4,4a,5,6,7,8,8a-octahydro-1H-isoquinolin-2-yl]-3-hydroxy-1-phenylbutan-2-yl]-2-(quinoline-2-carbonylamino)butanediamide; CAS Number 127779-20-8; chemical formula C₃₈H₅₀N₆O₅; and molar mass 670.8 g·mol-1. When present in an oral dissolvable film described herein, the saquinavir can function at least as an active pharmaceutical ingredient.

As used herein, the term “amprenavir” refers to the substance having the IUPAC name [(3S)-oxolan-3-yl] N-[(2S,3R)-4-[(4-aminophenyl)sulfonyl-(2-methylpropyl)amino]-3-hydroxy-1-phenylbutan-2-yl]carbamate; CAS Number 161814-49-9; chemical formula C₂₅H₃₅N₃O₆S; and molar mass 505.6 g·mol-1. When present in an oral dissolvable film described herein, the amprenavir can function at least as an active pharmaceutical ingredient.

As used herein, the term “valproic acid” refers to the substance having the IUPAC name 2-propylpentanoic acid; CAS Number 99-66-1; chemical formula C₈H₁₆O₂; and molar mass 144.21 g·mol-1. When present in an oral dissolvable film described herein, the valproic acid can function at least as an active pharmaceutical ingredient.

As used herein, the term “calcitriol” refers to the substance having the IUPAC name (1R,3S,5Z)-5-[(2E)-2-[(1R,3aS,7aR)-1-[(2R)-6-hydroxy-6-methylheptan-2-yl]-7α-methyl-2,3,3a,5,6,7-hexahydro-1H-inden-4-ylidene]ethylidene]-4-methylidenecyclohexane-1,3-diol; CAS Number 32222-06-3; chemical formula C₂₇H₄₄O₃; and molar mass 416.6 g·mol-1. When present in an oral dissolvable film described herein, the calcitriol can function at least as an active pharmaceutical ingredient.

As used herein, the term “bexarotene” refers to the substance having the IUPAC name 4-[1-(3,5,5,8,8-pentamethyl-6,7-dihydronaphthalen-2-yl)ethenyl]benzoic acid; CAS Number 153559-49-0; chemical formula C₂₄H₂₈O₂; and molar mass 348.5 g·mol-1. When present in an oral dissolvable film described herein, the bexarotene can function at least as an active pharmaceutical ingredient.

As used herein, the term “tretinoin” refers to the substance having the IUPAC name (2E,4E,6E,8E)-3,7-dimethyl-9-(2,6,6-trimethylcyclohexen-1-yl)nona-2,4,6,8-tetraenoic acid; CAS Number 302-79-4, 4759-48-2, 97950-17-9; chemical formula C₂₀H₂₈O₂; and molar mass 300.4 g·mol-1. When present in an oral dissolvable film described herein, the tretinoin can function at least as an active pharmaceutical ingredient.

As used herein, the term “isotretinoin” refers to the substance having the IUPAC name (2Z,4E,6E,8E)-3,7-dimethyl-9-(2,6,6-trimethylcyclohexen-1-yl)nona-2,4,6,8-tetraenoic acid; CAS Number 4759-48-2, 97950-17-9; chemical formula C₂₀H₂₈O₂; and molar mass 300.44 g·mol-1. When present in an oral dissolvable film described herein, the isotretinoin can function at least as an active pharmaceutical ingredient.

As used herein, the term “tipranavir” refers to the substance having the IUPAC name N-[3-[(1R)-1-[(2R)-4-hydroxy-6-oxo-2-(2-phenylethyl)-2-propyl-3H-pyran-5-yl]propyl]phenyl]-5-(trifluoromethyl)pyridine-2-sulfonamide; CAS Number 174484-41-4; chemical formula C₃₁H₃₃F₃N₂O₅S; and molar mass 602.7 g·mol-1. When present in an oral dissolvable film described herein, the tipranavir can function at least as an active pharmaceutical ingredient.

As used herein, the term “lysergic acid diethylamide (LSD)” refers to the substance having the IUPAC name (6aR,9R)—N,N-diethyl-7-methyl-6,6a,8,9-tetrahydro-4H-indolo[4,3-fg]quinoline-9-carboxamide; CAS Number 50-37-3; chemical formula C₂₀H₂₅N₃O; and molar mass 323.4 g·mol-1. When present in an oral dissolvable film described herein, the lysergic acid diethylamide (LSD) can function at least as an active pharmaceutical ingredient.

As used herein, the term “3,4-methylenedioxymethamphetamine (MDMA)” refers to the substance having the IUPAC name 1-(1,3-benzodioxol-5-yl)-N-methylpropan-2-amine; CAS Number 42542-10-9; chemical formula C₁₁H₁₅NO₂; and molar mass 193.24 g·mol-1. When present in an oral dissolvable film described herein, the 3,4-methylenedioxymethamphetamine (MDMA) can function at least as an active pharmaceutical ingredient.

As used herein, the term “N,N-Dimethyltryptamine (DMT)” refers to the substance having the IUPAC name 2-(1H-indol-3-yl)-N,N-dimethylethanamine; CAS Number 61-50-7; chemical formula C₁₂H₁₆N₂; and molar mass 188.269 g·mol-1. DMT is a chemical substance that occurs in many plants and animals and which is both a derivative and a structural analog of tryptamine. It can be consumed as a psychedelic drug and has historically been prepared by various cultures for ritual purposes as an entheogen. DMT is a functional analog and structural analog of other psychedelic tryptamines such as O-acetylpsilocin (4-AcO-DMT), 5-MeO-DMT, psilocybin (4-PO-DMT), psilocin (4-HO-DMT), and bufotenin (5-HO-DMT). The structure of DMT occurs within some important biomolecules like serotonin and melatonin, making them structural analogs of DMT. When present in an oral dissolvable film described herein, the N,N-Dimethyltryptamine (DMT) can function at least as an active pharmaceutical ingredient.

As used herein, the term “Psilocybin” refers to the substance with the IUPAC name [3-(2-dimethylaminoethyl)-1H-indol-4-yl] dihydrogen phosphate; CAS Number 520-52-5; chemical formula C₁₂H₁₇N₂O₄P; and molar mass 284.252 g·mol-1. Psilocybin is a naturally occurring psychedelic prodrug compound produced by more than 200 species of fungus. The most potent are members of the genus Psilocybe, such as P. azurescens, P. semilanceata, and P. cyanescens, but psilocybin has also been isolated from about a dozen other genera. As a prodrug, psilocybin is quickly converted by the body to psilocin, which has mind-altering effects similar, in some aspects, to those of LSD, mescaline, and DMT. In general, the effects include euphoria, visual and mental hallucinations, changes in perception, a distorted sense of time, and perceived spiritual experiences, and can also include possible adverse reactions such as nausea and panic attacks.

As used herein, the term “Mescaline” refers to the substance having the IUPAC name 2-(3,4,5-trimethoxyphenyl)ethanamine: CAS number 54-04-6; chemical formula C₁₁H₁₇NO₃; and molar mass 211.261 g·mol-1. Mescaline is a naturally occurring psychedelic protoalkaloid of the substituted phenethylamine class, known for its hallucinogenic effects comparable to those of LSD and psilocybin. It occurs naturally in the peyote cactus (Lophophora williamsii), the San Pedro cactus (Echinopsis pachanoi), the Peruvian torch (Echinopsis peruviana), and other species of cactus. It is also found in small amounts in certain members of the bean family, Fabaceae, including Acacia berlandieri.

As used herein, the term “Ibogaine” refers to a substance having the IUPAC name 12-methoxyibogamine; CAS number 83-74-9; chemical formula C₂₀H₂₆N₂O; and molar mass 310.441 g·mol-1. Ibogaine is a naturally occurring psychoactive substance found in plants in the family Apocynaceae such as Tabernanthe iboga, Voacanga africana, and Tabernaemontana undulata. It is a psychedelic with dissociative properties.

As used herein, the term “ivermectin” refers to the substance having the IUPAC name 22,23-dihydroavermectin B₁ₐ+22,23-dihydroavermectin Bib; CAS Number 70288-86-7 and 71827-03-7; chemical formula C₄H₇₄O₁₄ (22,23-dihydroavermectin B₁ₐ) and C₄₇H₇₂O₁₄ (22,23-dihydroavenmectin B_{1b}); and molar mass 875.106 g·mol-1 (22,23-dihydroavermectin B₁ₐ) and 861.079 g·mol-1 (22,23-dihydroavermectin B_{1b}). When present in an oral dissolvable film described herein, the ivermectin can function at least as an active pharmaceutical ingredient.

As used herein, the term “propylene glycol” refers to the substance having the IUPAC name propane-1,2-diol; CAS Number 57-55-6, 25322-68-3, 63625-56-9; chemical formula C₃H₈O₂ or CH₃CHOHCH₂OH; and molar mass 76.09 g·mol-1. When present in an oral dissolvable film described herein, the propylene glycol can function at least as a plasticizer.

As used herein, the term “glycerin” refers to the substance having the IUPAC name propane-1,2,3-triol; CAS Number 56-81-5, 8043-29-6, 25618-55-7, 8013-25-0; chemical formula C₃H₈O₃ or CH₂OH—CHOH—CH₂OH; and molar mass 92.09 g·mol-1. When present in an oral dissolvable film described herein, the glycerin can function at least as a plasticizer.

As used herein, the term “triacetin” refers to the substance having the IUPAC name 2,3-diacetyloxypropyl acetate; CAS Number 102-76-1; chemical formula C₉H₁₄O₆ or C₃H₅(OCOCH₃)₃; and molar mass 218.2 g·mol-1. When present in an oral dissolvable film described herein, the triacetin can function at least as a plasticizer.

As used herein, the term “triethyl citrate” refers to the substance having the IUPAC name triethyl 2-hydroxypropane-1,2,3-tricarboxylate; CAS Number 77-93-0; chemical formula C₁₂H₂₀O₇ or (CH₂COOC₂H₅)₂COHCOOC₂H₅; and molar mass 276.28 g·mol-1. When present in an oral dissolvable film described herein, the triethyl citrate can function at least as a plasticizer.

As used herein, the term “polyethylene glycol” refers to a polymer of the substance having the IUPAC name ethane-1,2-diol; CAS Number 107-21-1, 25322-68-3; chemical formula C₂H₆O₂ or (C₂H₄O)nH₂O (n=number of ethylene oxide units corresponding to a molecular weight of 6000, about 140) or HOCH₂CH₂OH or CH₂OHCH₂OH; and molar mass 62.07 g·mol-1. When present in an oral dissolvable film described herein, the polyethylene glycol can function at least as a plasticizer.

As used herein, the term “pullulan” refers to the substance having the IUPAC name [(2R,3S,4R,5R,6S)-4,5-dihydroxy-3-[(2R,3R,4S,5S,6R)-3,4,5-trihydroxy-6-(hydroxymethyl)oxan-2-yl]oxy-6-[[(2R,3S,4S,5R,6R)-3,4,5-trihydroxy-6-[(2R,3S,4R,5R,6S)-4,5,6-trihydroxy-2-(hydroxymethyl)oxan-3-yl]oxyoxan-2-yl]methoxy]oxan-2-yl]methyl hexadecanoate; CAS Number 53572-58-0; chemical formula C₄₀H₇₂O₂₂, and molar mass 905 g·mol-1. When present in an oral dissolvable film described herein, the pullulan can function at least as a film former.

As used herein, the term “gum arabic” refers to the substance extracted from Acacia Senegal having the IUPAC name 17-acetyl-3,7-dihydroxy-4,4,10,13,14-pentamethyl-2,3,5,6,7,12,16,17-octahydro-1H-cyclopenta[a]phenanthrene-11,15-dione; CAS Number 97653-92-4; chemical formula C₂₄H₃₄O₅; and molar mass 402.5 g·mol-1. When present in an oral dissolvable film described herein, the gum arabic can function at least as a film former.

As used herein, the term “guar gum” refers to the substance having the IUPAC name disodium;[[[5-(6-aminopurin-9-yl)-3-hydroxyoxolan-2-yl]oxy-methoxyphosphoryl]oxy-oxidophosphoryl] hydrogen phosphate; CAS Number 9000-30-0; chemical formula C₁₀H₁₄N₅Na₂O₁₂P₃; and molar mass 535.15 g·mol-1. When present in an oral dissolvable film described herein, the guar gum can function at least as a film former.

As used herein, the term “maltodextrin” refers to the substance having the IUPAC name (3R,4S,5S,6R)-2-[(2R,3S,4R,5R)-4,5-dihydroxy-2-(hydroxymethyl)-6-[(2R,3S,4R,5R,6S)-4,5,6-trihydroxy-2-(hydroxymethyl)oxan-3-yl]oxyoxan-3-yl]oxy-6-(hydroxymethyl)oxane-3,4,5-triol; CAS Number 9004-53-9; chemical formula C₁₈H₃₂O₁₆; and molar mass 504.4 g·mol-1. When present in an oral dissolvable film described herein, the maltodextrin can function at least as a film former.

As used herein, the term “microcrystalline cellulose” refers to the substance having the IUPAC name 2-[4,5-dihydroxy-2-(hydroxymethyl)-6-methoxyoxan-3-yl]oxy-6-(hydroxymethyl)-5-methoxyoxane-3,4-diol; CAS Number 9004-34-6; chemical formula C₁₄H₂₆O₁₁; and molar mass 370.35 g·mol-1. When present in an oral dissolvable film described herein, the microcrystalline cellulose can function at least as a film former.

As used herein, the term “chitosan” refers to the substance having the IUPAC name methyl N-[(2S,3R,4R,5S,6R)-5-[(2S,3R,4R,5S,6R)-3-amino-5-[(2S,3R,4R,5S,6R)-3-amino-5-[(2S,3R,4R,5S,6R)-3-amino-5-[(2S,3R,4R,5S,6R)-3-amino-5-[(2S,3R,4R,5S,6R)-3-amino-5-[(2S,3R,4R,5S,6R)-3-amino-4,5-dihydroxy-6-(hydroxymethyl)oxan-2-yl]oxy-4-hydroxy-6-(hydroxymethyl)oxan-2-yl]oxy-4-hydroxy-6-(hydroxymethyl)oxan-2-yl]oxy-4-hydroxy-6-(hydroxymethyl)oxan-2-yl]oxy-4-hydroxy-6-(hydroxymethyl)oxan-2-yl]oxy-4-hydroxy-6-(hydroxymethyl)oxan-2-yl]oxy-2-[(2R,3S,4R,5R,6S)-5-amino-6-[(2R,3S,4R,5R,6R)-5-amino-4,6-dihydroxy-2-(hydroxymethyl)oxan-3-yl]oxy-4-hydroxy-2-(hydroxymethyl)oxan-3-yl]oxy-4-hydroxy-6-(hydroxymethyl)oxan-3-yl]carbamate; CAS Number 9012-76-4; chemical formula C₅₆H₁₀₃N₉O₃₉; and molar mass 1526.5 g·mol-1. When present in an oral dissolvable film described herein, the chitosan can function at least as a film former.

As used herein, the term “pectin” refers to the substance having the IUPAC name (2S,3R,4S,5R,6R)-3,4,5,6-tetrahydroxyoxane-2-carboxylic acid; CAS Number 18968-14-4; chemical formula C₆H₁₀O₇; and molar mass 194.14 g·mol-1. When present in an oral dissolvable film described herein, the pectin can function at least as a film former.

As used herein, the term “carrageenan” refers to the substance having the IUPAC name zinc;1-(5-cyanopyridin-2-yl)-3-[(1S,2S)-2-(6-fluoro-2-hydroxy-3-propanoylphenyl)cyclopropyl]urea;diacetate; CAS Number 9000-07-1; chemical formula C₂₃H₂₃FN₄O₇Zn; and molar mass 551.8 g·mol-1. When present in an oral dissolvable film described herein, the carrageenan can function at least as a film former.

As used herein, the term “HPMC” or “hydroxypropyl methylcellulose” or “hypromellose” refers to a semisynthetic, inert, viscoelastic polymer having the CAS Number 9004-65-3. When present in an oral dissolvable film described herein, the HPMC can function at least as a film former.

As used herein, the term “HPC” or “hydroxypropyl cellulose” refers to the substance having the IUPAC name 1-[[(2R,3R,4S,5R)-3,4,5-tris(2-hydroxypropoxy)-6-[(2R,3R,4S,5R,6R)-4,5,6-tris(2-hydroxypropoxy)-2-(2-hydroxypropoxymethyl)oxan-3-yl]oxyoxan-2-yl]methoxy]propan-2-ol or analogs or derivatives thereof; CAS Number; chemical formula C₃₆H₇₀O₁₉; and molar mass 806.9 g·mol-1. When present in an oral dissolvable film described herein, the HPC can function at least as a film former.

As used herein, the term “modified corn starch” refers to the substance having the IUPAC name 5-[5-[3,4-dihydroxy-6-(hydroxymethyl)-5-methoxyoxan-2-yl]oxy-6-[[3,4-dihydroxy-6-(hydroxymethyl)-5-methoxyoxan-2-yl]oxymethyl]-3,4-dihydroxyoxan-2-yl]oxy-6-(hydroxymethyl)-2-methyloxane-3,4-diol or analogs or derivatives thereof; CAS Number; chemical formula C₂₇H₄₈O₂₀; and molar mass 692.7 g·mol-1. When present in an oral dissolvable film described herein, the modified corn starch can function at least as a film former.

As used herein, the term “carbopol 974P” refers to a member of the carbomer family including high molecular weight, crosslinked polyacrylic acid polymers or analogs of derivatives thereof. The carbopol includes but is not limited to, carbopol homopolymers: acrylic acid crosslinked with allyl sucrose or allyl pentaerythritol; carbopol copolymers: acrylic acid and C10-C30 alkyl acrylate crosslinked with allyl pentaerythritol; and carbopol interpolymers: carbomer homopolymer or copolymer that contains a block copolymer of polyethylene glycol and a long chain alkyl acid ester. When present in an oral dissolvable film described herein, the carbopol 974P can function at least as a film former.

As used herein, the term “carbopol 934P” refers to a member of the carbomer family including high molecular weight, crosslinked polyacrylic acid polymers or analogs of derivatives thereof. The carbopol includes but is not limited to, carbopol homopolymers: acrylic acid crosslinked with allyl sucrose or allyl pentaerythritol; carbopol copolymers: acrylic acid and C10-C30 alkyl acrylate crosslinked with allyl pentaerythritol; and carbopol interpolymers: carbomer homopolymer or copolymer that contains a block copolymer of polyethylene glycol and a long chain alkyl acid ester. When present in an oral dissolvable film described herein, the carbopol 934P can function at least as a film former.

As used herein, the term “kollidon 25” refers to a member of the polyvinylpyrrolidone family including high molecular weight, crosslinked polymers or analogs of derivatives thereof. When present in an oral dissolvable film described herein, the kollidon 25 can function at least as a film former.

As used herein, the term “soluplus” refers to the substance having the IUPAC name 2-hydroxyethyl 12-hydroxyoctadecanoate or analogs or derivatives thereof; CAS Number 105109-85-1, 6284-41-9; chemical formula C₂₀H₄₀O₄; and molar mass 344.5 g·mol-1. When present in an oral dissolvable film described herein, the soluplus can function at least as a film former.

As used herein, the term “lycoat NG73” refers to a member of the hydroxypropyl pea starch family such as polymers or analogs or derivatives thereof. When present in an oral dissolvable film described herein, the lycoat NG73 can function at least as a film former.

As used herein, the term “Kollicoat” or “Kollicoat®” refers to products commercially available from BASF (Florham Park, N.J.). These include, e.g., Kollicoat® Protect and Kollicoat® IR. The term “Kollicoat® Protect” refers to the commercial product containing (i) 35-45 wt. % polyvinyl alcohol (PVA), (ii) 55-65 wt. % polyvinyl alcohol (PVA)-polyethylene glycol (PEG) graft copolymer, and (iii) 0.1-0.3 wt. % silicon dioxide. Kollicoat® Protect is a combination of water-soluble Kollicoat® IR and polyvinyl alcohol, wherein the Kollicoat® IR is a polyvinyl alcohol (PVA)-polyethylene glycol (PEG) graft copolymer. The PEG portion of Kollicoat® IR is PEG 6000. Kollicoat® Protect has the chemical name polyvinyl alcohol-polyethylene glycol copolymer and polyvinyl alcohol (PVA). Kollicoat® Protect has the CAS-Nos: Kollicoat® IR 96734-39-3, Polyvinyl alcohol 9002-89-5, and silicon dioxide 7631-86-9.

When present in the oral dissolvable film described herein, the Kollicoat® product(s) can function at least as a film former.

As used herein, the term “polyox N-10” refers to a member of the polyoxalene family such as polymers of the substance having the IUPAC name 2-methyloxirane;oxirane or analogs or derivatives thereof; CAS Number 691397-13-4, 9003-11-6, 106392-12-5; chemical formula C5H₁₀02; and molar mass 102.13 g·mol-1. When present in an oral dissolvable film described herein, the polyox N-10 can function at least as a film former.

As used herein, the term “polyox N-80” refers to a member of the polyoxalene family such as polymers of the substance having the IUPAC name 2-methyloxirane;oxirane or analogs or derivatives thereof; CAS Number 691397-13-4, 9003-11-6, 106392-12-5; chemical formula C₅H₁₀O₂; and molar mass 102.13 g·mol-1. When present in an oral dissolvable film described herein, the polyox N-80 can function at least as a film former.

As used herein, the term “polyox N-750” refers to a member of the polyoxalene family such as polymers of the substance having the IUPAC name 2-methyloxirane;oxirane or analogs or derivatives thereof; CAS Number 691397-13-4, 9003-11-6, 106392-12-5; chemical formula C₅H₁₀O₂; and molar mass 102.13 g·mol-1. When present in an oral dissolvable film described herein, the polyox N-750 can function at least as a film former.

As used herein, the term “methocel E4M” refers to a member of the methylcellulose ether family such as polymers of the substance having the IUPAC name 6-(hydroxymethyl)-5-methoxy-2-[4,5,6-trimethoxy-2-(methoxymethyl)oxan-3-yl]oxyoxane-3,4-diol or analogs or derivatives thereof; CAS Number 99638-59-2; chemical formula C₁₇H₃₂O₁₁; and molar mass 412.4 g·mol-1. When present in an oral dissolvable film described herein, the methocel E4M can function at least as a film former.

As used herein, the term “methocel E10M” refers to a member of the methylcellulose ether family such as polymers of the substance having the IUPAC name 6-(hydroxymethyl)-5-methoxy-2-[4,5,6-trimethoxy-2-(methoxymethyl)oxan-3-yl]oxyoxane-3,4-diol or analogs or derivatives thereof; CAS Number 99638-59-2; chemical formula C₁₇H₃₂O₁₁; and molar mass 412.4 g·mol-1. When present in an oral dissolvable film described herein, the methocel E10M can function at least as a film former.

As used herein, the term “sodium CMC” or “sodium carboxymethyl cellulose” refers to the substance having the IUPAC name sodium;2,3,4,5,6-pentahydroxyhexanal:acetate or analogs or derivatives thereof; CAS Number 9004-32-4; chemical formula C₈H₁₅NaO₈; and molar mass 262.19 g·mol-1. When present in an oral dissolvable film described herein, the sodium CMC can function at least as a film former.

As used herein, the term “diethylene glycol monoethyl ether” refers to the substance having the IUPAC name 2-(2-ethoxyethoxy)ethanol; CAS Number 111-90-0; chemical formula C₆H₁₄O₃ or CH₃CH₂OCH₂CH₂OCH₂CH₂OH; and molar mass 134.17 g·mol-1. When present in an oral dissolvable film described herein, the diethylene glycol monoethyl ether can function at least as a film former.

As used herein, the term “caprylocapryol polyoxyl-8 glycerides” refers to the substance having the IUPAC name 2,3-dihydroxypropyl decanoate;2,3-dihydroxypropyl octanoate or analogs or derivatives thereof; CAS Number; chemical formula C₂₄H₄₈O₈; and molar mass 464.6 g·mol-1. When present in an oral dissolvable film described herein, the caprylocapryol polyoxyl-8 glycerides can function at least as a film former.

### SPECIFIC RANGES, VALUES, AND EMBODIMENTS

The specific embodiments describing the ranges and values provided below are for illustration purposes only, and do not otherwise limit the scope of the disclosed subject matter, as defined by the claims.

In specific embodiments, the oral dissolvable film includes an active pharmaceutical ingredient that is lipophilic or hydrophobic.

In specific embodiments, the oral dissolvable film includes an active pharmaceutical ingredient that is lipophilic and hydrophobic.

In specific embodiments, the oral dissolvable film includes a surfactant that is lipophilic or hydrophobic.

In specific embodiments, the oral dissolvable film includes a surfactant that is lipophilic and hydrophobic.

In specific embodiments, the oral dissolvable film includes a solvent, for the active pharmaceutical ingredient, wherein the solvent is lipophilic or hydrophobic.

In specific embodiments, the oral dissolvable film includes a solvent, for the active pharmaceutical ingredient, wherein the solvent is lipophilic and hydrophobic.

In specific embodiments, the oral dissolvable film includes: (1) an active pharmaceutical ingredient that is lipophilic or hydrophobic, (2) a surfactant that is lipophilic or hydrophobic, and (3) a solvent is lipophilic or hydrophobic.

In specific embodiments, the oral dissolvable film includes: (1) an active pharmaceutical ingredient that is lipophilic and hydrophobic, (2) a surfactant that is lipophilic and hydrophobic, and (3) a solvent is lipophilic and hydrophobic.

In specific embodiments, the oral dissolvable film includes an active pharmaceutical ingredient that is lipophobic or hydrophilic.

In specific embodiments, the oral dissolvable film includes an active pharmaceutical ingredient that is lipophobic and hydrophilic.

In specific embodiments, the oral dissolvable film includes a surfactant that is lipophobic or hydrophilic.

In specific embodiments, the oral dissolvable film includes a surfactant that is lipophobic and hydrophilic.

In specific embodiments, the oral dissolvable film includes a solvent for the active pharmaceutical ingredient, wherein the solvent is lipophobic or hydrophilic.

In specific embodiments, the oral dissolvable film includes a solvent for the active pharmaceutical ingredient, wherein the solvent is lipophobic and hydrophilic.

In specific embodiments, the oral dissolvable film includes: (1) an active pharmaceutical ingredient that is lipophobic or hydrophilic, (2) a surfactant that is lipophobic or hydrophilic, and (3) a solvent for the active pharmaceutical ingredient, wherein the solvent is lipophobic or hydrophilic.

In specific embodiments, the oral dissolvable film includes: (1) an active pharmaceutical ingredient that is lipophobic and hydrophilic, (2) a surfactant that is lipophobic and hydrophilic, and (3) a solvent for the active pharmaceutical ingredient, wherein the solvent is lipophobic and hydrophilic.

In specific embodiments, the surfactant is lipophilic or hydrophobic; and the solvent for the active pharmaceutical ingredient is lipophilic or hydrophobic.

In specific embodiments, the lipophilic or hydrophobic surfactant includes at least one of Glyceryl Monocaprylate, Propylene Glycol Monocaprylate, Glyceryl Monooleate, Propylene Glycol Monolaurate, Glyceryl Caprylate/Caprate, Glyceryl Monolinoleate, Sorbitan Monooleate (Span 80), Glyceryl Dibehenate, Propylene Glycol Dilaurate, Glyceryl Tricaprylate/Tricaprate, Glycerol Tricaprylate/Caprate, Decaglycerol Mono and Di Oleate, Oleoyl Macrogolglycerides, Lauroyl Macrogolglycerides, Stearoyl Macrogolglycerides or Stearoyl Polyoxylglycerides, and Polyoxyethylene Caprylic/Capric Glycerides.

In specific embodiments, the lipophilic or hydrophobic surfactant includes at least one of Glyceryl Monocaprylate, Propylene Glycol Monocaprylate, Glyceryl Monooleate, Propylene Glycol Monolaurate, Glyceryl Caprylate/Caprate, Glyceryl Monolinoleate, Sorbitan Monooleate (Span 80), Glyceryl Dibehenate, Propylene Glycol Dilaurate, Glyceryl Tricaprylate/Tricaprate, Glycerol Tricaprylate/Caprate, Decaglycerol Mono and Di Oleate, Oleoyl Macrogolglycerides, Lauroyl Macrogolglycerides, Stearoyl Macrogolglycerides, Stearoyl Polyoxylglycerides, Polyoxyethylene, and Caprylic/Capric Glycerides.

In specific embodiments, the lipophilic or hydrophobic surfactant includes at least one of Glyceryl Monocaprylate, Propylene Glycol Monocaprylate, Glyceryl Monooleate, Propylene Glycol Monolaurate, Glyceryl Monolinoleate, Sorbitan Monooleate (Span 80), Propylene Glycol Dilaurate, and Decaglycerol Mono and Di Oleate.

In specific embodiments, the lipophilic or hydrophobic surfactant includes at least one of Propylene Glycol Monocaprylate, Glyceryl Monooleate, Propylene Glycol Monolaurate, Glyceryl Monolinoleate, and Sorbitan Monooleate (Span 80).

In specific embodiments, the lipophilic or hydrophobic surfactant is present in 0.5-40 wt. %.

In specific embodiments, the lipophilic or hydrophobic surfactant is present in 3-25 wt. %.

In specific embodiments, the lipophilic or hydrophobic surfactant is present in 8-14 wt. %.

In specific embodiments, the lipophilic or hydrophobic surfactant is present in 11 wt. %.

In specific embodiments, the lipophilic or hydrophobic surfactant includes one or more substances as shown below. In further embodiments, the lipophilic or hydrophobic surfactant includes one or more substances in the amount/range, as shown below.

Lipophilic or hydrophobic surfactant

| Embodiment A | | Embodiment B | | Embodiment C | |
|---|---|---|---|---|---|
| Glyceryl | (i): 11% | Glyceryl | (i): 11% | | |
| Monocaprylate | (ii): 8-14% | Monocaprylate | (ii): 8-14% | | |
| | (iii): 3-25% | | (iii): 3-25% | | |
| | (iv): 0.5-40% | | (iv): 0.5-40% | | |
| Propylene Glycol | (i): 11% | Propylene Glycol | (i): 11% | Propylene Glycol | (i): 11% |
| Monocaprylate | (ii): 8-14% | Monocaprylate | (ii): 8-14% | Monocaprylate | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Glyceryl | (i): 11% | Glyceryl | (i): 11% | Glyceryl | (i): 11% |
| Monooleate | (ii): 8-14% | Monooleate | (ii): 8-14% | Monooleate | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Propylene Glycol | (i): 11% | Propylene Glycol | (i): 11% | Propylene Glycol | (i): 11% |
| Monolaurate | (ii): 8-14% | Monolaurate | (ii): 8-14% | Monolaurate | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Glyceryl Caprylate/ | (i): 11% | | | | |
| Caprate | (ii): 8-14% | | | | |
| | (iii): 3-25% | | | | |
| | (iv): 0.5-40% | | | | |
| Glyceryl | (i): 11% | Glyceryl | (i): 11% | Glyceryl | (i): 11% |
| Monolinoleate | (ii): 8-14% | Monolinoleate | (ii): 8-14% | Monolinoleate | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Sorbitan Monooleate | (i): 2% | Sorbitan Monooleate | (i): 2% | Sorbitan Monooleate | (i): 2% |
| (Span 80) | (ii): 1-3% | (Span 80) | (ii): 1-3% | (Span 80) | (ii): 1-3% |
| | (iii): 0.5-25% | | (iii): 0.5-25% | | (iii): 0.5-25% |
| | (iv): 0.5-40% | | (iv): 0.5-40% | | (iv): 0.5-40% |
| Glyceryl Dibehenate | (i): 5% | | | | |
| | (ii): 3-7% | | | | |
| | (iii): 0.5-25% | | | | |
| | (iv): 0.5-40% | | | | |
| Propylene Glycol | (i): 5% | Propylene Glycol | (i): 5% | | |
| Dilaurate | (ii): 3-7% | Dilaurate | (ii): 3-7% | | |
| | (iii): 0.5-25% | | (iii): 0.5-25% | | |
| | (iv): 0.5-40% | | (iv): 0.5-40% | | |
| Glyceryl | (i): 5% | | | | |
| Tricaprylate/ | (ii): 3-7% | | | | |
| Tricaprate | (iii): 0.5-25% | | | | |
| | (iv): 0.5-40% | | | | |
| Glycerol | (i): 5% | | | | |
| Tricaprylate/ | (ii): 3-7% | | | | |
| Caprate | (iii): 0.5-25% | | | | |
| | (iv): 0.5-40% | | | | |
| Decaglycerol Mono | (i): 5% | Decaglycerol Mono | (i): 5% | | |
| and Di Oleate | (ii): 3-7% | and Di Oleate | (ii): 3-7% | | |
| | (iii): 0.5-25% | | (iii): 0.5-25% | | |
| | (iv): 0.5-40% | | (iv): 0.5-40% | | |
| Oleoyl | (i): 5% | | | | |
| Macrogolglycerides | (ii): 3-7% | | | | |
| | (iii): 0.5-25% | | | | |
| | (iv): 0.5-40% | | | | |
| Lauroyl | (i): 5% | | | | |
| Macrogolglycerides | (ii): 3-7% | | | | |
| | (iii): 0.5-25% | | | | |
| | (iv): 0.5-40% | | | | |
| Stearoyl | (i): 5% | | | | |
| Macrogolglycerides | (ii): 3-7% | | | | |
| or Stearoyl | (iii): 0.5-25% | | | | |
| Polyoxylglycerides | (iv): 0.5-40% | | | | |
| Polyoxyethylene | (i): 11% | | | | |
| Caprylic/Capric | (ii): 8-14% | | | | |
| Glycerides | (iii): 3-25% | | | | |
| | (iv): 0.5-40% | | | | |

In specific embodiments, the surfactant is lipophobic or hydrophilic; and the solvent for the active pharmaceutical ingredient is lipophobic or hydrophilic.

In specific embodiments, the lipophobic or hydrophilic surfactant includes at least one of Poloxamer, Polyoxyl Castor Oil, Polyethylene-polypropylene Glycol, Polyoxyethylene Sorbitan Monolaurate (Tween 20), Tween 80, Polyoxyethylenesorbitan Monostearate (Tween 60), Decyl Glucoside, Lauryl Glucoside, Octyl Glucoside, Triton X-100, Nonoxynol 9, Sodium Lauryl Sulfate, Potassium Lauryl Sulfate, Brij, Glyceryl Laurate, Phospholipids, n-Dodecyl Phosphocholine, and Cholesteryl Esters.

In specific embodiments, the lipophobic or hydrophilic surfactant includes at least one of Poloxamer, Polyethylene-polypropylene Glycol, Polyoxyethylene Sorbitan Monolaurate (Tween 20), Tween 80, Polyoxyethylenesorbitan Monostearate (Tween 60), Triton X-100, Sodium Lauryl Sulfate, Brij, Phospholipids, n-Dodecyl Phosphocholine, and Cholesteryl Esters.

In specific embodiments, the lipophobic or hydrophilic surfactant includes at least one of Poloxamer, Polyethylene-polypropylene Glycol, Polyoxyethylene Sorbitan Monolaurate (Tween 20), Phospholipids, and n-Dodecyl Phosphocholine.

In specific embodiments, the lipophobic or hydrophilic surfactant includes at least one of Poloxamer, Polyoxyl Castor Oil, Polyethylene-polypropylene Glycol, Polyoxyethylene Sorbitan Monolaurate (Tween 20), Tween 80, Polyoxyethylenesorbitan Monostearate (Tween 60), Decyl Glucoside, Lauryl Glucoside, Octyl Glucoside, Triton X-100, Nonoxynol 9, Sodium Lauryl Sulfate, Potassium Lauryl Sulfate, Brij, Glyceryl Laurate, Phospholipids, n-Dodecyl Phosphocholine, and Cholesteryl Esters.

In specific embodiments, the lipophobic or hydrophilic surfactant is present in 0.5-40 wt. %.

In specific embodiments, the lipophobic or hydrophilic surfactant is present in 0.5-25 wt. %.

In specific embodiments, the lipophobic or hydrophilic surfactant is present in 3-7 wt. %.

In specific embodiments, the lipophobic or hydrophilic surfactant is present in 5 wt. %.

In specific embodiments, the lipophobic or hydrophilic surfactant includes one or more substances as shown below. In further embodiments, the lipophobic or hydrophilic surfactant includes one or more substances in the amount/range, as shown below.

Lipophobic or hydrophilic surfactant

| Embodiment A | | Embodiment B | | Embodiment C | |
|---|---|---|---|---|---|
| Poloxamer | (i): 5% | Poloxamer | (i): 5% | Poloxamer | (i): 5% |
| | (ii): 3-7% | | (ii): 3-7% | | (ii): 3-7% |
| | (iii): 0.5-25% | | (iii): 0.5-25% | | (iii): 0.5-25% |
| | (iv): 0.5-40% | | (iv): 0.5-40% | | (iv): 0.5-40% |
| Polyoxyl Castor | (i): 5% | | | | |
| Oil | (ii): 3-7% | | | | |
| | (iii): 0.5-25% | | | | |
| | (iv): 0.5-40% | | | | |
| Polyethylene- | (i): 5% | Polyethylene- | (i): 5% | Polyethylene- | (i): 5% |
| polypropylene | (ii): 3-7% | polypropylene | (ii): 3-7% | polypropylene | (ii): 3-7% |
| Glycol | (iii): 0.5-25% | Glycol | (iii): 0.5-25% | Glycol | (iii): 0.5-25% |
| | (iv): 0.5-40% | | (iv): 0.5-40% | | (iv): 0.5-40% |
| Polyoxyethylene | (i): 5% | Polyoxyethylene | (i): 5% | Polyoxyethylene | (i): 5% |
| Sorbitan | (ii): 3-7% | Sorbitan | (ii): 3-7% | Sorbitan | (ii): 3-7% |
| Monolaurate | (iii): 0.5-25% | Monolaurate | (iii): 0.5-25% | Monolaurate | (iii): 0.5-25% |
| (Tween 20) | (iv): 0.5-40% | (Tween 20) | (iv): 0.5-40% | (Tween 20) | (iv): 0.5-40% |
| Tween 80 | (i): 5% | Tween 80 | (i): 5% | | |
| | (ii): 3-7% | | (ii): 3-7% | | |
| | (iii): 0.5-25% | | (iii): 0.5-25% | | |
| | (iv): 0.5-40% | | (iv): 0.5-40% | | |
| Polyoxyethylene- | (i): 5% | Polyoxyethylene- | (i): 5% | | |
| sorbitan | (ii): 3-7% | sorbitan | (ii): 3-7% | | |
| Monostearate | (iii): 0.5-25% | Monostearate | (iii): 0.5-25% | | |
| (Tween 60) | (iv): 0.5-40% | (Tween 60) | (iv): 0.5-40% | | |
| Decyl | (i): 5% | | | | |
| Glucoside | (ii): 3-7% | | | | |
| | (iii): 0.5-25% | | | | |
| | (iv): 0.5-40% | | | | |
| Lauryl | (i): 5% | | | | |
| Glucoside | (ii): 3-7% | | | | |
| | (iii): 0.5-25% | | | | |
| | (iv): 0.5-40% | | | | |
| Octyl Glucoside | (i): 5% | | | | |
| | (ii): 3-7% | | | | |
| | (iii): 0.5-25% | | | | |
| | (iv): 0.5-40% | | | | |
| Triton X-100 | (i): 1% | Triton X-100 | (i): 1% | | |
| | (ii): 1-5% | | (ii): 1-5% | | |
| | (iii): 0.5-10% | | (iii): 0.5-10% | | |
| | (iv): 0.5-40% | | (iv): 0.5-40% | | |
| Nonoxynol 9 | (i): 1% | | | | |
| | (ii): 1-5% | | | | |
| | (iii): 0.5-10% | | | | |
| | (iv): 0.5-40% | | | | |
| Sodium Lauryl | (i): 0.5% | Sodium Lauryl | (i): 0.5% | | |
| Sulfate | (ii): 0.5-2% | Sulfate | (ii): 0.5-2% | | |
| | (iii): 0.5-5% | | (iii): 0.5-5% | | |
| | (iv): 0.5-10% | | (iv): 0.5-10% | | |
| Potassium | (i): 0.5% | | | | |
| Lauryl Sulfate | (ii): 0.5-2% | | | | |
| | (iii): 0.5-5% | | | | |
| | (iv): 0.5-10% | | | | |
| Brij | (i): 1% | Brij | (i): 1% | | |
| | (ii): 1-5% | | (ii): 1-5% | | |
| | (iii): 0.5-10% | | (iii): 0.5-10% | | |
| | (iv): 0.5-40% | | (iv): 0.5-40% | | |
| Glyceryl | (i): 1% | | | | |
| Laurate | (ii): 1-5% | | | | |
| | (iii): 0.5-10% | | | | |
| | (iv): 0.5-40% | | | | |
| Phospholipids | (i): 5% | Phospholipids | (i): 5% | Phospholipids | (i): 5% |
| | (ii): 3-7% | | (ii): 3-7% | | (ii): 3-7% |
| | (iii): 0.5-25% | | (iii): 0.5-25% | | (iii): 0.5-25% |
| | (iv): 0.5-40% | | (iv): 0.5-40% | | (iv): 0.5-40% |
| n-Dodecyl | (i): 0.5% | n-Dodecyl | (i): 0.5% | n-Dodecyl | (i): 0.5% |
| Phosphocholine | (ii): 0.5-2% | Phosphocholine | (ii): 0.5-2% | Phosphocholine | (ii): 0.5-2% |
| | (iii): 0.5-5% | | (iii): 0.5-5% | | (iii): 0.5-5% |
| | (iv): 0.5-10% | | (iv): 0.5-10% | | (iv): 0.5-10% |
| Cholesteryl | (i): 0.5% | Cholesteryl | (i): 0.5% | | |
| Esters | (ii): 0.5-2% | Esters | (ii): 0.5-2% | | |
| | (iii): 0.5-5% | | (iii): 0.5-5% | | |
| | (iv): 0.5-10% | | (iv): 0.5-10% | | |

In specific embodiments, the lipophilic or hydrophobic solvent for the active pharmaceutical ingredient includes at least one of Medium Chain Triglycerides Oil, Coconut Oil, Corn Oil, Olive Oil, Palm Oil, Canola Oil, Safflower Oil, Sesame Oil, Propylene Glycol Monocaprylate, Propylene Glycol Monolaurate, Glyceryl Monolinoleate, Cetyl Alcohol, Stearyl Alcohol, Cetostearyl Alcohol, and Oleyl Alcohols.

In specific embodiments, the lipophilic or hydrophobic solvent for the active pharmaceutical ingredient includes at least one of Medium Chain Triglycerides Oil, Coconut Oil, Olive Oil, Sesame Oil, Propylene Glycol Monocaprylate, Propylene Glycol Monolaurate, and Glyceryl Monolinoleate.

In specific embodiments, the lipophilic or hydrophobic solvent for the active pharmaceutical ingredient includes at least one of Medium Chain Triglycerides Oil, Olive Oil, Sesame Oil, Propylene Glycol Monocaprylate, Propylene Glycol Monolaurate, and Glyceryl Monolinoleate.

In specific embodiments, the lipophilic or hydrophobic solvent for the active pharmaceutical ingredient includes at least one of Medium Chain Triglycerides Oil, Coconut Oil, Corn Oil, Olive Oil, Palm Oil, Canola Oil, Safflower Oil, Sesame Oil, Propylene Glycol Monocaprylate, Propylene Glycol Monolaurate, Glyceryl Monolinoleate, Cetyl Alcohol, Stearyl Alcohol, Cetostearyl Alcohol, and Oleyl Alcohols.

In specific embodiments, the lipophilic or hydrophobic solvent for the active pharmaceutical ingredient is present in 0.5-40 wt. %.

In specific embodiments, the lipophilic or hydrophobic solvent for the active pharmaceutical ingredient is present in 3-25 wt. %.

In specific embodiments, the lipophilic or hydrophobic solvent for the active pharmaceutical ingredient is present in 8-14 wt. %.

In specific embodiments, the lipophilic or hydrophobic solvent for the active pharmaceutical ingredient is present in 11 wt. %.

In specific embodiments, the lipophobic or hydrophilic solvent for the active pharmaceutical ingredient includes water.

In specific embodiments, the lipophobic or hydrophilic solvent for the active pharmaceutical ingredient is present in 0.5-20 wt. %.

In specific embodiments, the lipophobic or hydrophilic solvent for the active pharmaceutical ingredient includes one or more substances as shown below. In further embodiments, the lipophobic or hydrophilic solvent for the active pharmaceutical ingredient includes one or more substances in the amount/range, as shown below.

Lipophilic or hydrophobic solvent for the active ingredient/Oil Carrier

| Embodiment A | | Embodiment B | | Embodiment C | |
|---|---|---|---|---|---|
| Medium Chain | (i): 11% | Medium Chain | (i): 11% | Medium Chain | (i): 11% |
| Triglycerides | (ii): 8-14% | Triglycerides | (ii): 8-14% | Triglycerides | (ii): 8-14% |
| Oil | (iii): 3-25% | Oil | (iii): 3-25% | Oil | (iii): 3-25% |
| | (iv): 0.5-40% | | (iv): 0.5-40% | | (iv): 0.5-40% |
| Coconut Oil | (i): 11% | Coconut Oil | (i): 11% | | |
| | (ii): 8-14% | | (ii): 8-14% | | |
| | (iii): 3-25% | | (iii): 3-25% | | |
| | (iv): 0.5-40% | | (iv): 0.5-40% | | |
| Corn Oil | (i): 11% | | | | |
| | (ii): 8-14% | | | | |
| | (iii): 3-25% | | | | |
| | (iv): 0.5-40% | | | | |
| Olive Oil | (i): 11% | Olive Oil | (i): 11% | Olive Oil | (i): 11% |
| | (ii): 8-14% | | (ii): 8-14% | | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Palm Oil | (i): 11% | | | | |
| | (ii): 8-14% | | | | |
| | (iii): 3-25% | | | | |
| | (iv): 0.5-40% | | | | |
| Canola Oil | (i): 11% | | | | |
| | (ii): 8-14% | | | | |
| | (iii): 3-25% | | | | |
| | (iv): 0.5-40% | | | | |
| Safflower Oil | (i): 11% | | | | |
| | (ii): 8-14% | | | | |
| | (iii): 3-25% | | | | |
| | (iv): 0.5-40% | | | | |
| Sesame Oil | (i): 11% | Sesame Oil | (i): 11% | Sesame Oil | (i): 11% |
| | (ii): 8-14% | | (ii): 8-14% | | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Propylene | (i): 11% | Propylene | (i): 11% | Propylene | (i): 11% |
| Glycol | (ii): 8-14% | Glycol | (ii): 8-14% | Glycol | (ii): 8-14% |
| Monocaprylate | (iii): 3-25% | Monocaprylate | (iii): 3-25% | Monocaprylate | (iii): 3-25% |
| | (iv): 0.5-40% | | (iv): 0.5-40% | | (iv): 0.5-40% |
| Propylene | (i): 11% | Propylene | (i): 11% | Propylene | (i): 11% |
| Glycol | (ii): 8-14% | Glycol | (ii): 8-14% | Glycol | (ii): 8-14% |
| Monolaurate | (iii): 3-25% | Monolaurate | (iii): 3-25% | Monolaurate | (iii): 3-25% |
| | (iv): 0.5-40% | | (iv): 0.5-40% | | (iv): 0.5-40% |
| Glyceryl | (i): 11% | Glyceryl | (i): 11% | Glyceryl | (i): 11% |
| Monolinoleate | (ii): 8-14% | Monolinoleate | (ii): 8-14% | Monolinoleate | (ii): 8-14% |
| | (iii): 3-25% | | (iii): 3-25% | | (iii): 3-25% |
| | (iv): 0.5-40% | | (iv): 0.5-40% | | (iv): 0.5-40% |
| Cetyl Alcohol | (i): 11% | | | | |
| | (ii): 8-14% | | | | |
| | (iii): 3-25% | | | | |
| | (iv): 0.5-40% | | | | |
| Stearyl Alcohol | (i): 11% | | | | |
| | (ii): 8-14% | | | | |
| | (iii): 3-25% | | | | |
| | (iv): 0.5-40% | | | | |
| Cetostearyl | (i): 11% | | | | |
| Alcohol | (ii): 8-14% | | | | |
| | (iii): 3-25% | | | | |
| | (iv): 0.5-40% | | | | |
| Oleyl Alcohols | (i): 11% | | | | |
| | (ii): 8-14% | | | | |
| | (iii): 3-25% | | | | |
| | (iv): 0.5-40% | | | | |

In specific embodiments, the active pharmaceutical ingredient is lipophilic or hydrophobic.

In specific embodiments, the active pharmaceutical ingredient is lipophobic or hydrophilic.

In specific embodiments, the active pharmaceutical ingredient includes a cannabinoid, terpene, flavonoid, or combination thereof.

In specific embodiments, the flavonoid includes FBL-03G.

In specific embodiments, the active pharmaceutical ingredient includes at least one of cyclosporine, ritonavir, saquinavir, amprenavir, valproic acid, calcitriol, bexarotene, tretinoin, isotretinoin, tipranavir, and pharmaceutically acceptable salts thereof.

In specific embodiments, the active pharmaceutical ingredient includes a psychedelic agent.

In specific embodiments, the active pharmaceutical ingredient includes a psychedelic agent including at least one of Lysergic acid diethylamide (LSD) and 3,4-Methylenedioxy methamphetamine (MDMA).

In specific embodiments, the active pharmaceutical ingredient includes ivermectin.

In specific embodiments, the active pharmaceutical ingredient is present in 0.5-40 wt. %.

In specific embodiments, the active pharmaceutical ingredient is present in 0.5-30 wt. %.

In specific embodiments, the active pharmaceutical ingredient is present in 0.5-20 wt. %.

In specific embodiments, the active pharmaceutical ingredient is present in 0.5-10 wt. %.

In specific embodiments, the active pharmaceutical ingredient is present in at least 10 wt. %.

In specific embodiments, the active pharmaceutical ingredient is present in 10-40 wt. %.

In specific embodiments, the active pharmaceutical ingredient is present in 10-35 wt. %.

In specific embodiments, the active pharmaceutical ingredient is present in 10-30 wt. %.

In specific embodiments, the active pharmaceutical ingredient is present in 10-25 wt. %.

In specific embodiments, the active pharmaceutical ingredient is present in 10-20 wt. %.

In specific embodiments, the active pharmaceutical ingredient is present in 0.01-5 wt. %.

In specific embodiments, the active pharmaceutical ingredient is present in 0.01-2.5 wt. %.

In specific embodiments, the active pharmaceutical ingredient is present in 0.01-1.0 wt. %.

In specific embodiments, the active pharmaceutical ingredient is present in 0.01-0.5 wt. %.

In specific embodiments, the active pharmaceutical ingredient is present in up to 2.5 wt. %.

In specific embodiments, the active pharmaceutical ingredient is present in up to 1.5 wt. %.

In specific embodiments, the active pharmaceutical ingredient is present in up to 1.0 wt. %.

In specific embodiments, the active pharmaceutical ingredient is present in up to 0.5 wt. %.

In specific embodiments, the film matrix of the oral dissolvable film includes a plasticizer and film former.

In specific embodiments, the film matrix of the oral dissolvable film includes a plasticizer including at least one of Propylene Glycol, Glycerin, Triacetin, Triethyl Citrate, and Polyethylene Glycol.

In specific embodiments, the film matrix of the oral dissolvable film includes a plasticizer including at least one of Propylene Glycol, Glycerin, and Polyethylene Glycol.

In specific embodiments, the film matrix of the oral dissolvable film includes a plasticizer including at least one of Glycerin and Polyethylene Glycol.

In specific embodiments, the film matrix of the oral dissolvable film includes a plasticizer present in 0.5-20 wt. %.

In specific embodiments, the film matrix of the oral dissolvable film includes a plasticizer present in 3-20 wt. %.

In specific embodiments, the film matrix of the oral dissolvable film includes a plasticizer present in 8-14 wt. %.

In specific embodiments, the film matrix of the oral dissolvable film includes a plasticizer present in 12 wt. %.

In specific embodiments, the plasticizer includes one or more substances as shown below. In further embodiments, the plasticizer includes one or more substances in the amount/range, as shown below.

Plasticizer

| Embodiment A | | Embodiment B | | Embodiment C | |
|---|---|---|---|---|---|
| Propylene | (i): 12% | Propylene | (i): 12% | | |
| Glycol | (ii): 8-14% | Glycol | (ii): 8-14% | | |
| | (iii): 3-20% | | (iii): 3-20% | | |
| | (iv): 0.5-20% | | (iv): 0.5-20% | | |
| Glycerin | (i): 12% | Glycerin | (i): 12% | Glycerin | (i): 12% |
| | (ii): 8-14% | | (ii): 8-14% | | (ii): 8-14% |
| | (iii): 3-20% | | (iii): 3-20% | | (iii): 3-20% |
| | (iv): 0.5-20% | | (iv): 0.5-20% | | (iv): 0.5-20% |
| Triacetin | (i): 3% | | | | |
| | (ii): 1-5% | | | | |
| | (iii): 3-20% | | | | |
| | (iv): 0.5-20% | | | | |
| Triethyl Citrate | (i): 3% | | | | |
| | (ii): 1-5% | | | | |
| | (iii): 3-20% | | | | |
| | (iv): 0.5-20% | | | | |
| Polyethylene | (i): 10% | Polyethylene | (i): 10% | Polyethylene | (i): 10% |
| Glycol | (ii): 7-13% | Glycol | (ii): 7-13% | Glycol | (ii): 7-13% |
| | (iii): 3-20% | | (iii): 3-20% | | (iii): 3-20% |
| | (iv): 0.5-20% | | (iv): 0.5-20% | | (iv): 0.5-20% |

In specific embodiments, the film matrix of the oral dissolvable film includes a film former including at least one of Pullulan, Gum Arabic, Guar Gum, Maltodextrin, Microcrystalline Cellulose, Chitosan, Pectin, Carrageenan, HPMC, HPC, Modified Corn Starch, Carbopol 974P, Carbopol 934P, Kollidon 25, Soluplus, Lycoat NG73, Kollicoat, Polyox N-10, Polyox N-80, Polyox N-750, Methocel E4M, Methocel E10M, and Sodium CMC.

In specific embodiments, the film matrix of the oral dissolvable film includes a film former including at least one of Pullulan, Gum Arabic, Microcrystalline Cellulose, Chitosan, Pectin, Carrageenan, HPMC, Modified Corn Starch, Kollidon 25, and Soluplus.

In specific embodiments, the film matrix of the oral dissolvable film includes a film former including at least one of Pullulan, Microcrystalline Cellulose, Chitosan, Pectin, HPMC, Modified Corn Starch, Kollidon 25, and Soluplus.

In specific embodiments, the film matrix of the oral dissolvable film includes a film former present in 1-60 wt. %.

In specific embodiments, the film matrix of the oral dissolvable film includes a film former present in 5-40 wt. %.

In specific embodiments, the film matrix of the oral dissolvable film includes a film former present in 10-20 wt. %.

In specific embodiments, the film matrix of the oral dissolvable film includes a film former present in 1-20 wt. %.

In specific embodiments, the film matrix of the oral dissolvable film includes a film former present in 3-7 wt. %.

In specific embodiments, the film matrix of the oral dissolvable film includes a film former present in 14 wt. %.

In specific embodiments, the film matrix of the oral dissolvable film includes a film former present in 5 wt. %.

In specific embodiments, the film former includes one or more substances as shown below. In further embodiments, the film former includes one or more substances in the amount/range, as shown below.

Film former

| Embodiment A | | Embodiment B | | Embodiment C | |
|---|---|---|---|---|---|
| Pullulan | (i): 14% | Pullulan | (i): 14% | Pullulan | (i): 14% |
| | (ii): 10-20% | | (ii): 10-20% | | (ii): 10-20% |
| | (iii): 5-40% | | (iii): 5-40% | | (iii): 5-40% |
| | (iv): 1-60% | | (iv): 1-60% | | (iv): 1-60% |
| Gum Arabic | (i): 5% | Gum Arabic | (i): 5% | | |
| | (ii): 3-7% | | (ii): 3-7% | | |
| | (iii): 1-20% | | (iii): 1-20% | | |
| | (iv): 1-60% | | (iv): 1-60% | | |
| Guar Gum | (i): 5% | | | | |
| | (ii): 3-7% | | | | |
| | (iii): 1-20% | | | | |
| | (iv): 1-60% | | | | |
| Maltodextrin | (i): 5% | | | | |
| | (ii): 3-7% | | | | |
| | (iii): 1-20% | | | | |
| | (iv): 1-60% | | | | |
| Microcrystalline | (i): 5% | Microcrystalline | (i): 5% | Microcrystalline | (i): 5% |
| Cellulose | (ii): 3-7% | Cellulose | (ii): 3-7% | Cellulose | (ii): 3-7% |
| | (iii): 1-20% | | (iii): 1-20% | | (iii): 1-20% |
| | (iv): 1-60% | | (iv): 1-60% | | (iv): 1-60% |
| Chitosan | (i): 5% | Chitosan | (i): 5% | Chitosan | (i): 5% |
| | (ii): 3-7% | | (ii): 3-7% | | (ii): 3-7% |
| | (iii): 1-20% | | (iii): 1-20% | | (iii): 1-20% |
| | (iv): 1-60% | | (iv): 1-60% | | (iv): 1-60% |
| Pectin | (i): 14% | Pectin | (i): 14% | Pectin | (i): 14% |
| | (ii): 10-20% | | (ii): 10-20% | | (ii): 10-20% |
| | (iii): 5-40% | | (iii): 5-40% | | (iii): 5-40% |
| | (iv): 1-60% | | (iv): 1-60% | | (iv): 1-60% |
| Carrageenan | (i): 5% | Carrageenan | (i): 5% | | |
| | (ii): 3-7% | | (ii): 3-7% | | |
| | (iii): 1-20% | | (iii): 1-20% | | |
| | (iv): 1-60% | | (iv): 1-60% | | |
| HPMC | (i): 5% | HPMC | (i): 5% | HPMC | (i): 5% |
| | (ii): 3-7% | | (ii): 3-7% | | (ii): 3-7% |
| | (iii): 1-20% | | (iii): 1-20% | | (iii): 1-20% |
| | (iv): 1-60% | | (iv): 1-60% | | (iv): 1-60% |
| HPC | (i): 5% | | | | |
| | (ii): 3-7% | | | | |
| | (iii): 1-20% | | | | |
| | (iv): 1-60% | | | | |
| Modified Corn | (i): 5% | Modified Corn | (i): 5% | Modified Corn | (i): 5% |
| Starch | (ii): 3-7% | Starch | (ii): 3-7% | Starch | (ii): 3-7% |
| | (iii): 1-20% | | (iii): 1-20% | | (iii): 1-20% |
| | (iv): 1-60% | | (iv): 1-60% | | (iv): 1-60% |
| Carbopol 974P | (i): 5% | | | | |
| | (ii): 3-7% | | | | |
| | (iii): 1-20% | | | | |
| | (iv): 1-60% | | | | |
| Carbopol 934P | (i): 5% | | | | |
| | (ii): 3-7% | | | | |
| | (iii): 1-20% | | | | |
| | (iv): 1-60% | | | | |
| Kollidon 25 | (i): 5% | Kollidon 25 | (i): 5% | Kollidon 25 | (i): 5% |
| | (ii): 3-7% | | (ii): 3-7% | | (ii): 3-7% |
| | (iii): 1-20% | | (iii): 1-20% | | (iii): 1-20% |
| | (iv): 1-60% | | (iv): 1-60% | | (iv): 1-60% |
| Soluplus | (i): 14% | Soluplus | (i): 14% | Soluplus | (i): 14% |
| | (ii): 10-20% | | (ii): 10-20% | | (ii): 10-20% |
| | (iii): 5-40% | | (iii): 5-40% | | (iii): 5-40% |
| | (iv): 1-60% | | (iv): 1-60% | | (iv): 1-60% |
| Lycoat NG73 | (i): 5% | | | | |
| | (ii): 3-7% | | | | |
| | (iii): 1-20% | | | | |
| | (iv): 1-60% | | | | |
| Kollicoat | (i): 5% | | | | |
| | (ii): 3-7% | | | | |
| | (iii): 1-20% | | | | |
| | (iv): 1-60% | | | | |
| Polyox N-10 | (i): 5% | | | | |
| | (ii): 3-7% | | | | |
| | (iii): 1-20% | | | | |
| | (iv): 1-60% | | | | |
| Polyox N-80 | (i): 5% | | | | |
| | (ii): 3-7% | | | | |
| | (iii): 1-20% | | | | |
| | (iv): 1-60% | | | | |
| Polyox N-750 | (i): 5% | | | | |
| | (ii): 3-7% | | | | |
| | (iii): 1-20% | | | | |
| | (iv): 1-60% | | | | |
| Methocel E4M | (i): 5% | | | | |
| | (ii): 3-7% | | | | |
| | (iii): 1-20% | | | | |
| | (iv): 1-60% | | | | |
| Methocel E10M | (i): 5% | | | | |
| | (ii): 3-7% | | | | |
| | (iii): 1-20% | | | | |
| | (iv): 1-60% | | | | |
| Sodium CMC | (i): 5% | | | | |
| | (ii): 3-7% | | | | |
| | (iii): 1-20% | | | | |
| | (iv): 1-60% | | | | |

In specific embodiments, the oral dissolvable film further includes a co-solvent.

In specific embodiments, the oral dissolvable film further includes a co-solvent that includes at least one of Diethylene Glycol Monoethyl Ether and Caprylocapryol Polyoxyl-8 Glycerides.

In specific embodiments, the oral dissolvable film further includes a co-solvent present in 0.5-40 wt. %.

In specific embodiments, the oral dissolvable film further includes a co-solvent present in 0.5-25 wt. %.

In specific embodiments, the oral dissolvable film further includes a co-solvent present in 3-7 wt. %.

In specific embodiments, the oral dissolvable film further includes a co-solvent present in 5 wt. %.

In specific embodiments, the co-surfactant includes one or more substances as shown below. In further embodiments, the co-surfactant includes one or more substances in the amount/range, as shown below.

Co-surfactant

| Embodiment A | | Embodiment B | | Embodiment C | |
|---|---|---|---|---|---|
| Diethylene | (i): 5% | Diethylene | (i): 5% | Diethylene | (i): 5% |
| Glycol | (ii): 3-7% | Glycol | (ii): 3-7% | Glycol | (ii): 3-7% |
| Monoethyl | (iii): 0.5-25% | Monoethyl | (iii): 0.5-25% | Monoethyl | (iii): 0.5-25% |
| Ether | (iv): 0.5-40% | Ether | (iv): 0.5-40% | Ether | (iv): 0.5-40% |
| Caprylocapryol | (i): 5% | Caprylocapryol | (i): 5% | Caprylocapryol | (i): 5% |
| Polyoxyl-8 | (ii): 3-7% | Polyoxyl-8 | (ii): 3-7% | Polyoxyl-8 | (ii): 3-7% |
| Glycerides | (iii): 0.5-25% | Glycerides | (iii): 0.5-25% | Glycerides | (iii): 0.5-25% |
| | (iv): 0.5-40% | | (iv): 0.5-40% | | (iv): 0.5-40% |

In specific embodiments, the oral dissolvable film further includes at least one of an antioxidant, antimicrobial agent, flavoring agent, coloring agent, and sweetener.

In specific embodiments, the oral dissolvable film is configured to self-emulsify within 120 seconds upon contact with an oral mucosal surface of a subject.

In specific embodiments, the oral dissolvable film is configured to self-emulsify within 100 seconds upon contact with an oral mucosal surface of a subject.

In specific embodiments, the oral dissolvable film is configured to self-emulsify within 90 seconds upon contact with an oral mucosal surface of a subject.

In specific embodiments, the oral dissolvable film is configured to self-emulsify within 75 seconds upon contact with an oral mucosal surface of a subject.

In specific embodiments, the oral dissolvable film is configured to self-emulsify within 60 seconds upon contact with an oral mucosal surface of a subject.

In specific embodiments, the oral dissolvable film is configured to self-emulsify within 45 seconds upon contact with an oral mucosal surface of a subject.

In specific embodiments, the oral dissolvable film is configured to self-emulsify within 30 seconds upon contact with an oral mucosal surface of a subject.

In specific embodiments, the oral dissolvable film is configured to self-emulsify within 20 seconds upon contact with an oral mucosal surface of a subject.

In specific embodiments, the oral dissolvable film is configured to form an oil-in-water (O/W) emulsion within 120 seconds upon contact with an oral mucosal surface of a subject.

In specific embodiments, the oral dissolvable film is configured to form an oil-in-water (O/W) emulsion within 100 seconds upon contact with an oral mucosal surface of a subject.

In specific embodiments, the oral dissolvable film is configured to form an oil-in-water (O/W) emulsion within 90 seconds upon contact with an oral mucosal surface of a subject.

In specific embodiments, the oral dissolvable film is configured to form an oil-in-water (O/W) emulsion within 75 seconds upon contact with an oral mucosal surface of a subject.

In specific embodiments, the oral dissolvable film is configured to form an oil-in-water (O/W) emulsion within 60 seconds upon contact with an oral mucosal surface of a subject.

In specific embodiments, the oral dissolvable film is configured to form an oil-in-water (O/W) emulsion within 45 seconds upon contact with an oral mucosal surface of a subject.

In specific embodiments, the oral dissolvable film is configured to form an oil-in-water (O/W) emulsion within 30 seconds upon contact with an oral mucosal surface of a subject.

In specific embodiments, the oral dissolvable film is configured to form an oil-in-water (O/W) emulsion within 20 seconds upon contact with an oral mucosal surface of a subject.

In specific embodiments, the oral dissolvable film is configured to form an oil-in-water (O/W) emulsion having an average droplet size of 0.1 microns to 120 microns within 20 seconds upon contact with an oral mucosal surface of a subject.

In specific embodiments, the oral dissolvable film is configured to form an oil-in-water (O/W) emulsion having an average droplet size of: d(10): 0.5-10 micron, d(50): 1-20 micron, and d(90): 15-100 micron; within 20 seconds upon contact with an oral mucosal surface of a subject.

In specific embodiments, the oral dissolvable film is configured to form an oil-in-water (O/W) emulsion having an average droplet size of: d(10): 0.5-5 micron, d(50): 1-10 micron, and d(90): 15-50 micron.

In specific embodiments, the oral dissolvable film is configured to form an oil-in-water (O/W) emulsion having an average droplet size of: d(10): 0.5-2 micron, d(50): 1-5 micron, and d(90): 15-30 micron.

In specific embodiments, the oral dissolvable film is configured to form an oil-in-water (O/W) emulsion having an average droplet size as shown below.

Average droplet size oil-in-water (O/W)
emulsion. d(10), d(50), and d(90)

| Embodiment A | Embodiment B | Embodiment C |
|---|---|---|
| d(10): 0.5-10 micron | d(10): 0.5-5 micron | d(10): 0.5-2 micron |
| d(50): 1-20 micron | d(50): 1-10 micron | d(50): 1-5 micron |
| d(90): 15-100 micron | d(90): 15-50 micron | d(90): 15-30 micron |

In specific embodiments, the oral dissolvable film is suitable for oral administration (PO), buccal administration, sublingual administration, or mucosal administration.

In specific embodiments, the oral dissolvable film has a moisture content of 3-13 wt. %.

In specific embodiments, the oral dissolvable film has a moisture content of 5-13 wt. %.

In specific embodiments, the oral dissolvable film has a moisture content of 5-12 wt. %.

In specific embodiments, the oral dissolvable film has a moisture content of 5-11 wt. %.

In specific embodiments, the oral dissolvable film has a moisture content of 5-10 wt. %.

In specific embodiments, the oral dissolvable film has a moisture content of 5-9 wt. %.

In specific embodiments, the oral dissolvable film has a moisture content of 6-13 wt. %.

In specific embodiments, the oral dissolvable film has a moisture content of 6-12 wt. %.

In specific embodiments, the oral dissolvable film has a moisture content of 6-11 wt. %.

In specific embodiments, the oral dissolvable film has a moisture content of 6-10 wt. %.

In specific embodiments, the oral dissolvable film configured to disintegrate within 20 minutes upon buccal administration to a subject.

In specific embodiments, the oral dissolvable film configured to disintegrate within 15 minutes upon buccal administration to a subject.

In specific embodiments, the oral dissolvable film configured to disintegrate within 10 minutes upon buccal administration to a subject.

In specific embodiments, the oral dissolvable film configured to disintegrate within 5 minutes upon buccal administration to a subject.

In specific embodiments, the oral dissolvable film is configured to disintegrate within 120 seconds upon oral (PO) administration to a subject.

In specific embodiments, the oral dissolvable film is configured to disintegrate within 100 seconds upon oral (PO) administration to a subject.

In specific embodiments, the oral dissolvable film is configured to disintegrate within 90 seconds upon oral (PO) administration to a subject.

In specific embodiments, the oral dissolvable film is configured to disintegrate within 70 seconds upon oral (PO) administration to a subject.

In specific embodiments, the oral dissolvable film is configured to disintegrate within 60 seconds upon oral (PO) administration to a subject.

In specific embodiments, the oral dissolvable film is configured to disintegrate within 45 seconds upon oral (PO) administration to a subject.

In specific embodiments, the oral dissolvable film is configured to disintegrate within 30 seconds upon oral (PO) administration to a subject.

In specific embodiments, the oral dissolvable film is configured to disintegrate within 20 seconds upon oral (PO) administration to a subject.

In specific embodiments, the oral dissolvable film is configured for in vitro disintegration (USP<701> In-vitro Disintegration method) within 120 seconds.

In specific embodiments, the oral dissolvable film is configured for in vitro disintegration (USP<701> In-vitro Disintegration method) within 100 seconds.

In specific embodiments, the oral dissolvable film is configured for in vitro disintegration (USP<701> In-vitro Disintegration method) within 90 seconds.

In specific embodiments, the oral dissolvable film is configured for in vitro disintegration (USP<701> In-vitro Disintegration method) within 75 seconds.

In specific embodiments, the oral dissolvable film is configured for in vitro disintegration (USP<701> In-vitro Disintegration method) within 60 seconds.

In specific embodiments, the oral dissolvable film is configured for in vitro disintegration (USP<701> In-vitro Disintegration method) within 40 seconds.

In specific embodiments, the oral dissolvable film is configured for in vitro disintegration (USP<701> In-vitro Disintegration method) within 30 seconds.

In specific embodiments, the oral dissolvable film is configured for in vitro disintegration (USP<701> In-vitro Disintegration method) within 20 seconds.

In specific embodiments, the oral dissolvable film exhibits at least one pharmacokinetic parameter selected from, (i) Tmax of between about 45 min to about 120 min, (ii) Cmax of at least 3.5 ng/ml, and (iii) AUC₀₋ₜ of at least 13 ng/hr/ml.

In specific embodiments, the oral dissolvable film exhibits at least one pharmacokinetic parameter selected from, (i) Tmax of 1.5 hr, (ii) Cmax of 4.4 ng/ml, and (iii) AUC₀₋ₜ of 13.5 ng/hr/ml.

In specific embodiments, the oral dissolvable film exhibits an in vivo dissolution time of no more than 20 minutes.

In specific embodiments, the oral dissolvable film exhibits an in vivo dissolution time of between about 10 minutes to about 15 minutes.

In specific embodiments, the oral dissolvable film exhibits a bioavailability of at least 10%.

In specific embodiments, the oral dissolvable film exhibits a bioavailability of at least 12.5%.

In specific embodiments, the oral dissolvable film exhibits a bioavailability of at least 15%.

In specific embodiments, the oral dissolvable film exhibits a bioavailability of at least 18%.

In specific embodiments, the oral dissolvable film exhibits a bioavailability of at least 20%.

In specific embodiments, the oral dissolvable film exhibits a bioavailability of at least 25%.

In specific embodiments, the oral dissolvable film exhibits a stability of at least about 96% after nine months as measured under 40° C./75% RH accelerated conditions.

In specific embodiments, the oral dissolvable film exhibits a stability of 100% after three months as measured under 25° C./60% RH accelerated condition, or 40° C./75% RH accelerated conditions.

In specific embodiments, with the method of forming an oral dissolvable film, the film forming ingredient includes at least one of mucoadhesive polymer, plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, solvent, permeation enhancer, and preservative.

In specific embodiments, with the method of forming an oral dissolvable film, the lipophilic or hydrophobic solvent includes an oil.

In specific embodiments, with the method of forming an oral dissolvable film, the hydrophilic or lipophobic solvent includes an aqueous liquid.

In specific embodiments, with the method of forming an oral dissolvable film, the curing is carried out in a hot air oven at an air temperature of between about 38° C. to about 110° C.

In specific embodiments, with the method of forming an oral dissolvable film, the curing is carried out in a hot air oven at an air temperature of between about 45° C. to about 80° C.

In specific embodiments, with the method of forming an oral dissolvable film, the curing is carried out in a hot air oven (at an air temperature of 50° C.-70° C.).

In specific embodiments, with the method of forming an oral dissolvable film, the curing is carried out at a speed of between about 0.8 feet/min to about 2.5 feet/min.

In specific embodiments, with the method of forming an oral dissolvable film, the curing is carried out at a speed of between about 0.8 feet/min to about 1.0 feet/min.

In specific embodiments, with the method of forming an oral dissolvable film, the curing is carried out at a speed of between about 2.0 feet/min to about 2.5 feet/min.

### ENUMERATED EMBODIMENTS

Specific enumerated embodiments [1] to [57] provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment [1]

The present invention provides for an oral dissolvable film including:

(a) active pharmaceutical ingredient;

(b) surfactant;

(c) solvent for the active pharmaceutical ingredient;

(d) film matrix; and

(e) water;

wherein,

when the active pharmaceutical ingredient is lipophilic or hydrophobic: (i) the surfactant is lipophilic or hydrophobic, and (ii) the solvent for the active pharmaceutical ingredient is lipophilic or hydrophobic; and

when the active pharmaceutical ingredient is lipophobic or hydrophilic: (i) the surfactant is lipophobic or hydrophilic, and (ii) the solvent for the active pharmaceutical ingredient is lipophobic or hydrophilic.

### Embodiment [2]

The present invention provides for an oral dissolvable film of Embodiment [1], wherein the surfactant is lipophilic or hydrophobic and the solvent for the active pharmaceutical ingredient is lipophilic or hydrophobic.

### Embodiment [3]

The present invention provides for an oral dissolvable film of any one of Embodiments [1] to [2], wherein the lipophilic or hydrophobic surfactant includes at least one of Glyceryl Monocaprylate, Propylene Glycol Monocaprylate, Glyceryl Monooleate, Propylene Glycol Monolaurate, Glyceryl Caprylate/Caprate, Glyceryl Monolinoleate, Sorbitan Monooleate (Span 80), Glyceryl Dibehenate, Propylene Glycol Dilaurate, Glyceryl Tricaprylate/Tricaprate, Glycerol Tricaprylate/Caprate, Decaglycerol Mono and Di Oleate, Oleoyl Macrogolglycerides, Lauroyl Macrogolglycerides, Stearoyl Macrogolglycerides, Stearoyl Polyoxylglycerides, Polyoxyethylene, and Caprylic/Capric Glycerides.

### Embodiment [4]

The present invention provides for an oral dissolvable film of any one of Embodiments [1] to [3], wherein the lipophilic or hydrophobic surfactant is present in 0.5-40 wt. %.

### Embodiment [5]

The present invention provides for an oral dissolvable film of Embodiment [1], wherein the surfactant is lipophobic or hydrophilic, and the solvent for the active pharmaceutical ingredient is lipophobic or hydrophilic.

### Embodiment [6]

The present invention provides for an oral dissolvable film of any one of Embodiments [1] and [5], wherein the lipophobic or hydrophilic surfactant includes at least one of Poloxamer, Polyoxyl Castor Oil, Polyethylene-polypropylene Glycol, Polyoxyethylene Sorbitan Monolaurate (Tween 20), Tween 80, Polyoxyethylenesorbitan Monostearate (Tween 60), Decyl Glucoside, Lauryl Glucoside, Octyl Glucoside, Triton X-100, Nonoxynol 9, Sodium Lauryl Sulfate, Potassium Lauryl Sulfate, Brij, Glyceryl Laurate, Phospholipids, n-Dodecyl Phosphocholine, and Cholesteryl Esters.

### Embodiment [7]

The present invention provides for an oral dissolvable film of any one of Embodiments [1] and [5] to [6], wherein the lipophobic or hydrophilic surfactant is present in 0.5-40 wt. %.

### Embodiment [8]

The present invention provides for an oral dissolvable film of any one of Embodiments [1] to [3], wherein the lipophilic or hydrophobic solvent for the active pharmaceutical ingredient includes at least one of Medium Chain Triglycerides Oil, Coconut Oil, Corn Oil, Olive Oil, Palm Oil, Canola Oil, Safflower Oil, Sesame Oil, Propylene Glycol Monocaprylate, Propylene Glycol Monolaurate, Glyceryl Monolinoleate, Cetyl Alcohol, Stearyl Alcohol, Cetostearyl Alcohol, and Oleyl Alcohols.

### Embodiment [9]

The present invention provides for an oral dissolvable film of any one of Embodiments [1] to [3] and [8], wherein the lipophilic or hydrophobic solvent for the active pharmaceutical ingredient is present in 0.5-40 wt. %.

### Embodiment [10]

The present invention provides for an oral dissolvable film of any one of Embodiments [1] and [5] to [7], wherein the lipophobic or hydrophilic solvent for the active pharmaceutical ingredient includes water.

### Embodiment [11]

The present invention provides for an oral dissolvable film of any one of Embodiments [1], [5] to [7], and [10], wherein the lipophobic or hydrophilic solvent for the active pharmaceutical ingredient is present in 0.5-20 wt. %.

### Embodiment [12]

The present invention provides for an oral dissolvable film of any one of Embodiments [1] to [11], wherein the active pharmaceutical ingredient is lipophilic or hydrophobic.

### Embodiment [13]

The present invention provides for an oral dissolvable film of any one of Embodiments [1] to [11], wherein the active pharmaceutical ingredient is lipophobic or hydrophilic.

### Embodiment [14]

The present invention provides for an oral dissolvable film of any one of Embodiments [1] to [13], wherein the active pharmaceutical ingredient includes a cannabinoid, terpene, flavonoid, or combination thereof.

### Embodiment [15]

The present invention provides for an oral dissolvable film of any one of Embodiments [1] to [13], wherein the active pharmaceutical ingredient includes at least one of cyclosporine, ritonavir, saquinavir, amprenavir, valproic acid, calcitriol, bexarotene, tretinoin, isotretinoin, tipranavir, and pharmaceutically acceptable salts thereof.

### Embodiment [16]

The present invention provides for an oral dissolvable film of any one of Embodiments [1] to [13], wherein the active pharmaceutical ingredient includes a psychedelic agent.

### Embodiment [17]

The present invention provides for an oral dissolvable film of any one of Embodiments [1] to [13], wherein the active pharmaceutical ingredient includes a psychedelic agent that includes at least one of Lysergic acid diethylamide (LSD); 3,4-Methylenedioxymethamphetamine (MDMA); N,N-Dimethyltryptamine (DMT); Psilocybin, Mescaline, and Ibogaine.

### Embodiment [18]

The present invention provides for an oral dissolvable film of any one of Embodiments [1] to [13], wherein the active pharmaceutical ingredient includes ivermectin.

### Embodiment [19]

The present invention provides for an oral dissolvable film of any one of Embodiments [1] to [18], including the active pharmaceutical ingredient in at least 10 wt. %.

### Embodiment [20]

The present invention provides for an oral dissolvable film of any one of Embodiments [1] to [19], wherein the film matrix includes a plasticizer, and film former.

### Embodiment [21]

The present invention provides for an oral dissolvable film of any one of Embodiments [1] to [20], wherein the film matrix includes a plasticizer including at least one of Propylene Glycol, Glycerin, Triacetin, Triethyl Citrate, and Polyethylene Glycol.

### Embodiment [22]

The present invention provides for an oral dissolvable film of any one of Embodiments [1] to [21], wherein the film matrix includes a plasticizer present in 0.5-20 wt. %.

### Embodiment [23]

The present invention provides for an oral dissolvable film of any one of Embodiments [1] to [23], wherein the film matrix includes a film former including at least one of Pullulan, Gum Arabic, Guar Gum, Maltodextrin, Microcrystalline Cellulose, Chitosan, Pectin, Carrageenan, HPMC, HPC, Modified Corn Starch, Carbopol 974P, Carbopol 934P, Kollidon 25, Soluplus, Lycoat NG73, Kollicoat, Polyox N-10, Polyox N-80, Polyox N-750, Methocel E4M, Methocel E10M, and Sodium CMC.

### Embodiment [24]

The present invention provides for an oral dissolvable film of any one of Embodiments [1] to [23], wherein the film matrix includes a film former present in 1-60 wt. %.

### Embodiment [25]

The present invention provides for an oral dissolvable film of any one of Embodiments [1] to [24], further including a co-solvent.

### Embodiment [26]

The present invention provides for an oral dissolvable film of any one of Embodiments [1] to [25], further including a co-solvent including at least one of Diethylene Glycol Monoethyl Ether and Caprylocapryol Polyoxyl-8 Glycerides.

### Embodiment [27]

The present invention provides for an oral dissolvable film of any one of Embodiments [1] to [26], further including a co-solvent present in 0.5-40 wt. %.

### Embodiment [28]

The present invention provides for an oral dissolvable film of any one of Embodiments [1] to [27], further including at least one of an antioxidant, antimicrobial agent, flavoring agent, coloring agent, and sweetener.

### Embodiment [29]

The present invention provides for an oral dissolvable film of Embodiment [1], that includes:

(a) lipophilic active pharmaceutical ingredient,

(b) oil carrier for the lipophilic active pharmaceutical ingredient;

(c) self-emulsifying lipophilic surfactant for the lipophilic active pharmaceutical ingredient;

(d) one or more co-surfactants;

(e) one or more hydrophilic surfactants;

(f) film matrix, and

(g) water.

### Embodiment [30]

The present invention provides for an oral dissolvable film of Embodiment [1], that includes:

(a) hydrophilic active pharmaceutical ingredient;

(b) water carrier for the hydrophilic active pharmaceutical ingredient;

(c) hydrophilic surfactant for the hydrophilic active pharmaceutical ingredient;

(d) one or more co-surfactants;

(e) one or more self-emulsifying surfactants;

(f) film matrix; and

(g) water.

### Embodiment [31]

The present invention provides for an oral dissolvable film of any one of Embodiments [1] to [30], configured to self-emulsify within 20 seconds upon contact with an oral mucosal surface of a subject.

### Embodiment [32]

The present invention provides for an oral dissolvable film of any one of Embodiments [1] to [31], configured to form an oil-in-water (O/W) emulsion within 20 seconds upon contact with an oral mucosal surface of a subject.

### Embodiment [33]

The present invention provides for an oral dissolvable film of any one of Embodiments [1] to [32], configured to form an oil-in-water (O/W) emulsion having an average droplet size of 0.1 microns to 120 microns within 20 seconds upon contact with an oral mucosal surface of a subject.

### Embodiment [34]

The present invention provides for an oral dissolvable film of any one of Embodiments [1] to [33], configured to form an oil-in-water (O/W) emulsion having an average droplet size of

d(10): 0.5-10 micron,
d(50): 1-20 micron, and
d(90): 15-100 micron
within 20 seconds upon contact with an oral mucosal surface of a subject.

### Embodiment [35]

The present invention provides for an oral dissolvable film of any one of Embodiments [1] to [34], suitable for oral administration (PO), buccal administration, sublingual administration, or mucosal administration.

### Embodiment [36]

The present invention provides for an oral dissolvable film of any one of Embodiments [1] to [35], having a moisture content of 3-13 wt. %.

### Embodiment [37]

The present invention provides for an oral dissolvable film of any one of Embodiments [1] to [36], configured to disintegrate within 15 minutes upon buccal administration to a subject.

### Embodiment [38]

The present invention provides for an oral dissolvable film of any one of Embodiments [1] to [36], configured to disintegrate within 30 seconds upon oral (PO) administration to a subject.

### Embodiment [39]

The present invention provides for an oral dissolvable film of any one of Embodiments [1] to [36], configured for in vitro disintegration (USP<701> In-vitro Disintegration method) within 30 seconds.

### Embodiment [40]

The present invention provides for an oral dissolvable film of any one of Embodiments [1] to [39], exhibiting at least one pharmacokinetic parameter selected from, (i) Tmax of between about 45 min to about 120 min, (ii) Cmax of at least 3.5 ng/ml, and (iii) AUC₀₋ₜ of at least 13 ng/hr/ml.

### Embodiment [41]

The present invention provides for an oral dissolvable film of any one of Embodiments [1] to [40], exhibiting at least one pharmacokinetic parameter selected from, (i) Tmax of 1.5 hr, (ii) Cmax of 4.4 ng/ml, and (iii) AUC₀₋ₜ of 13.5 ng/hr/ml.

### Embodiment [42]

The present invention provides for an oral dissolvable film of any one of Embodiments [1] to [41], exhibiting an in vivo dissolution time of no more than 20 minutes.

### Embodiment [43]

The present invention provides for an oral dissolvable film of any one of Embodiments [1] to [42], exhibiting an in vivo dissolution time of between about 10 minutes to about 15 minutes.

### Embodiment [44]

The present invention provides for an oral dissolvable film of any one of Embodiments [1] to [43], exhibiting a bioavailability of at least 15%.

### Embodiment [45]

The present invention provides for an oral dissolvable film of any one of Embodiments [1] to [43], exhibiting a bioavailability of at least 18%.

### Embodiment [46]

The present invention provides for an oral dissolvable film of Embodiments [1] to [45], exhibiting a stability of at least about 96% after nine months as measured under 40° C./75% RH accelerated conditions.

### Embodiment [47]

The present invention provides for an oral dissolvable film of Embodiments [1] to [45], exhibiting a stability of 100% after three months as measured under 25° C./60% RH accelerated condition, or 40° C./75% RH accelerated conditions.

### Embodiment [48]

The present invention provides for a method of forming an oral dissolvable film, the method including:
(a) dissolving an active pharmaceutical ingredient in a first solvent-system to form a first mixture, wherein:
   (i) when the active pharmaceutical ingredient is lipophilic or hydrophobic, dissolving the active pharmaceutical ingredient in a lipophilic or hydrophobic solvent, in a lipophilic or hydrophobic surfactant, or combination thereof; or
   (ii) when the active pharmaceutical ingredient is hydrophilic or lipophobic, dissolving the active pharmaceutical ingredient in a hydrophilic or lipophobic solvent, in a hydrophilic or lipophobic surfactant, or combination thereof;
(b) contacting the first mixture and a lipophilic or hydrophobic surfactant to form a second mixture;
(c) contacting the second mixture with water and a hydrophilic or lipophobic surfactant to form a third mixture;
(d) contacting the third mixture with film forming ingredient to form a slurry; and
(e) casting the slurry on a substrate and curing to form the oral dissolvable film.

### Embodiment [49]

The present invention provides for a method of forming an oral dissolvable film of Embodiment [48], wherein the film forming ingredient includes at least one of mucoadhesive polymer, plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, solvent, permeation enhancer, and preservative.

### Embodiment [50]

The present invention provides for a method of forming an oral dissolvable film of any one of Embodiments [48] to [49], wherein the lipophilic or hydrophobic solvent includes an oil.

### Embodiment [51]

The present invention provides for a method of forming an oral dissolvable film of any one of Embodiments [48] to [49], wherein the hydrophilic or lipophobic solvent includes an aqueous liquid.

### Embodiment [52]

The present invention provides for a method of forming an oral dissolvable film of any one of Embodiments [48] to [51], wherein the curing is carried out in a hot air oven at an air temperature of between about 38° C. to about 110° C.

### Embodiment [53]

The present invention provides for a method of forming an oral dissolvable film of any one of Embodiments [48] to [52], wherein the curing is carried out in a hot air oven at an air temperature of between about 45° C. to about 80° C.

### Embodiment [54]

The present invention provides for a method of forming an oral dissolvable film of any one of Embodiments [48] to [53], wherein the curing is carried out in a hot air oven (at an air temperature of 50° C.-70° C.).

### Embodiment [55]

The present invention provides for a method of forming an oral dissolvable film of any one of Embodiments [48] to [54], wherein the curing is carried out at a speed of between about 0.8 feet/min to about 2.5 feet/min.

### Embodiment [56]

The present invention provides for a method of forming an oral dissolvable film of any one of Embodiments [48] to [55], wherein the curing is carried out at a speed of between about 0.8 feet/min to about 1.0 feet/min.

### Embodiment [57]

The present invention provides for a method of forming an oral dissolvable film of any one of Embodiments [48] to [56], wherein the curing is carried out at a speed of between about 2.0 feet/min to about 2.5 feet/min.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention. The invention can also be described by reference to the below examples and experimentals, which do not otherwise limit the scope of the invention.

### EXAMPLES AND EXPERIMENTALS

The formulation of the dissolvable film can include the active ingredient and polymer. The formulation of thin films can be challenged by the following factors (1-3): (1) the lack of stability of certain active ingredients can complicate the formulation of an oral thin film (OTF) or other thin films; (2) low bioavailability of active ingredients; or (3) low permeability of active ingredients.

With respect to factor (1), the presence of heat, moisture, light, and/or oxygen can degrade active ingredients that are sensitive to heat, moisture, light, or oxygen. An oral thin film (OTF) system can exhibit: (i) polymorphic transition of the active ingredient; (ii) hydration of polymers of the formulation containing the active ingredient; and (iii) decomposition and/or oxidation of the active ingredient via photolytic or hydrolytic processes. Hygroscopicity (i.e., adsorbing or absorbing water) is a factor impacting the formulation of thin films. Adsorbed or absorbed moisture in the thin film can impact mechanical strength, adhesion properties, and friability of the thin film. In addition to the hygroscopicity of the active ingredient, water levels during the formulation of the thin film can be elevated from: (i) polymers and solvents used to dissolve the polymer; and (ii) manufacturing techniques. The stability of the active ingredient can be further impacted by: (i) the amount of heat applied to dry the film used during manufacturing techniques; and (ii) the duration of drying time (i.e., the amount of time wet thin film is exposed to heat for drying).

With respect to factor (2), the use of thin films includes challenges such as: (i) low drug loading capacity for less potent drugs administered in a high dose: or (ii) potent drugs with less bioavailability.

With respect to factor (3), the mucus layer covering the epithelial cells, filters and limits the penetration of the epithelial cells by substances, such as small molecule drug active ingredients. Additionally, the thickness of the mucus layer slows down the diffusion of substances.

### EXEMPLARY ADVANTAGES OF THE INVENTION

The systems and methods described herein, are directed to self-emulsifying thin films containing the active ingredient. The formulations of self-emulsifying thin film provide the following advantages, in specific embodiments: (1) an increased barrier to moisture, oxygen, light, pH, and heat and thereby conferring protection to the active ingredient against moisture, oxygen, light, pH, and heat; (2) an improved bioavailability of less potent and less bioavailable active ingredients which allows the less potent active ingredients to be used at low doses; (3) possible reduction in liver/GI toxicity: and (4) an increased penetration and crossing of the mucus layer by the active ingredients and thereby allowing active ingredients to enter into systemic circulation.

With respect to advantage 3, the active ingredients of the self-emulsifying thin film are administered by (i) facilitating binding receptors for transport via enterocyte (i.e., transcellular processes); or (ii) loosening tightened junctions between cells for: (a) transport between cells and (b) transport of small molecule drug active ingredients for systemic circulation (i.e., paracellular processes). The systems and methods described herein, provide advantages of the thin film that can be obtained as a specific combination or in a singular fashion.

### Specific Combination 1

The systems and methods described herein, are characterized with higher bioavailability where a thin film can self-emulsify rapidly upon: (i) contact with a solvent in an oral cavity, and (ii) gentle agitation provided by the mouth of the patient consuming the thin film. There is a formation of a fine oil/water (o/w) emulsion. For buccal administration of a thin film and to a lesser extent oral administration, i.e., Per Os (PO), there is increased bioavailability and increased permeability of the active ingredients released from the self-emulsifying thin film. Additionally, the film matrix of the self-emulsifying thin film has a mucoadhesive property that allows for direct absorption of the active ingredient through the oral cavity into the blood.

### Specific Combination 2

The systems and methods described herein, are characterized with higher stability where an OTF can self-emulsify. The formulation of the self-emulsifying OTF has a thermo-gelling property and thus yielding physically stable formulations. A formulation of a self-emulsifying OTF which can protect the active ingredient against degradation to exposure to high temperatures is created in response to: (i) heat exposure during the gelatinization process of forming the self-emulsifying OTF, (ii) emulsification of ingredients, and (iii) subsequent cooling.

Specific Example with Vitamin D3 as the Active Ingredient

Vitamin D3 is a highly sensitive Lipophilic active pharmaceutical ingredient (API). It was used as a model drug, formulated using above formulation and, Vitamin D3 Oral Thin Film tested for 4 weeks stability study. In normal circumstances, Vitamin D3 degradation triggered at high heat and humidity, with this formulation we observed significant protection of Vitamin D3 in OTF and stability.

| | | Time Points And % Assay of Vitamin D3 | | | | |
|---|---|---|---|---|---|---|
| | Stability | T0 | T 1 | T2 | T3 | T4 |
| Drug | Condition | Week | Week | Weeks | Weeks | Weeks |
| Vitamin D3 | 25 C./ | 100% | 95% | 100% | 101% | 101% |
| | 60% RH | | | | | |
| | 40 C./ | | 98% | 92% | 91% | 93% |
| | 75% RH | | | | | |

Specific Example with Cannabidiol (CBD) as the Active Ingredient

Similarly, Active ingredient Cannabinoid CBD was tested for T=3M stability. We observed better stability and significant protection of the API in the film formulation.

| | | Time Points And % Assay of CBD | | | | |
|---|---|---|---|---|---|---|
| | Stability | T0 | T 2 | T1 | T2 | T3 |
| Drug | Condition | Week | Weeks | Month | Months | Months |
| CBD | 25 C./ | 108% | 109% | 109% | 113% | 120% |
| | 60% RH | | | | | |
| | 40 C./ | | 108% | 106% | 113% | 118% |
| | 75% RH | | | | | |

### Singular Fashion

The thin film containing a drug as an active ingredient can self-emulsify to provide: (i) more consistent temporal profiles of drug absorption, (ii) selective drug targeting toward a specific absorption window in the gastrointestinal (GI) tract; and (iii) protection of the drug from degradation in the gut. The gut can be acidic and impose harsh conditions that can biochemically breakdown the drug (i.e., degradation). More specifically, a drug which is a lipophilic compound, exhibits dissolution rate limited absorption. The thin film containing the drug may offer: (i) an increased rate and extent of absorption of the drug; and (ii) more reproducible blood time profiles. The thin film containing active ingredients that can self-emulsify, wherein the active ingredients are lipophilic, lipophobic, hydrophilic, or hydrophobic.

Upon disintegrating the self-emulsifying thin film containing the active ingredient, the active ingredient is released into the oral cavity and turned into o/w emulsion. More specifically, the active ingredient passes rapidly through the oral cavity, which can facilitate wide distribution of the active ingredient, such as a small molecule drug, throughout oral cavity or the GI tract. Thereby, the disintegrated self-emulsifying thin film can minimize the irritation frequently encountered during extended contact between bulk drug substance and the gut/oral wall.

### Bioavailability Data.

| | T1 (PO) % | T2 (Buccal) % | Ref % |
|---|---|---|---|
| by geometric mean | 18.4327295 | 15.25773908 | 10.44376624 |

Further advantages of the systems and methods described herein, as directed to thin films also include avoiding: (i) the need for water/beverage to swallow a pill; (ii) drug exposure to stomach acids and tissue irritation, (iii) drug absorption through the intestines; and (iv) liver metabolism and potential injury.

### PREFERRED EMBODIMENTS

If Active ingredient is Lipophilic:

1) Lipophilic Active ingredient with Oil carrier and one self-emulsifying lipophilic Surfactant

2) One or more co surfactants and Hydrophilic Surfactants

3) A film matrix creating ingredients

4) Water

If Active ingredient is Hydrophilic:

1) Hydrophilic Active ingredient in water with Hydrophilic Surfactant

2) One or more co surfactants and Self-emulsifying Surfactant

3) A film matrix creating ingredients

4) Water

The thin films of the systems and methods described herein, utilize: (i) at least one self-emulsifying surfactant; (ii) one or more co-surfactants; (iii) an oil or water solution containing an active ingredient; and (iv) a matrix. The active ingredient can be an active pharmaceutical ingredient, lipophilic active ingredient, hydrophilic active ingredient. The thin film contains a self-emulsifying system which is embedded into a film forming system.

More specifically, the thin films have the following features: (1) absence of emulsions in response to embedding the active ingredient into the film; (2) a matrix deriving from an initial emulsion and components for the construction of the film; and (3) an active ingredient protected by the matrix.

With respect to feature (2) above, the steps below are carried out to yield the matrix and in turn the thin film.

Step 1: Dissolve the active ingredient in a suitable solvent-system to yield Mix 1 where:
a) if the active ingredient is lipophilic, then dissolve the lipophilic active ingredient in the oil carrier or lipophilic surfactant; or
b) if the active ingredient is hydrophilic, then dissolve the hydrophilic active ingredient in water or water containing hydrophilic surfactant.
   Step 2: Add lipophilic surfactant into Mix 1 to yield Mix 2.
   Step 3: Add water with hydrophilic surfactant to yield Mix 3.
   Step 4: Add forming ingredients to Mix 3 to yield a slurry in a wet yet homogenous state, wherein the ingredients comprise: water, matrix forming ingredients, plasticizers, flavoring agents, and coloring agents. When the slurry is in a wet state, the active ingredient is covered by lipophilic and/or hydrophilic surfactant systems.
   Step 5: Cast the slurry and drying the slurry in a hot air oven (38° C.-110° C.) to yield a thin film.
   Step 6: After drying the thin film, perform peeling, cutting, and packing of the thin film and thereby obtaining strips deriving from the thin film. Upon drying of the thin film, the lipophilic and hydrophilic surfactants and film forming ingredients construct a matrix, which provides protection to the active ingredient and helps to improve stability of the thin film.

The matrix can be a gel deriving from a gelation phenomenon. The gelation phenomenon is due to interactions between hydrophobic polymer chains. By elevating temperatures, the hydrophobic polymer chains start to aggregate into a micelle structure. The formation of the micelle structure is the result of dehydration of the hydrophobic repeat units in the hydrophobic polymers chains. This gelation phenomenon can be reversible and characterized by a solution-gel transition temperature (Tsol-gel).

If the temperature of the thin film is below Tsol-gel, self-emulsifying hydrophilic-hydrophobic surfactants remain in a fluid state. If the temperature of the thin film is above Tsol-gel, solution phase material in the slurry turns into a semisolid material.

The systems and methods described herein, increase the following properties (1 and 2): (1) the stability of the thin films; and (2) bioavailability of the active ingredient. In turn, more active ingredient can be released to reach the target area upon dissolution of the thin film. The systems and methods described herein, obviate the need to compensate for degradation or loss of the active ingredient in the GI. Based on properties 1 and 2, the systems and methods described herein, can: (i) reduce the amount of active ingredient needed by individuals consuming the thin film; (ii) decrease the amount of possible side effects from the consuming the active ingredient; and (iii) reduce the cost of manufacturing thin film containing the active ingredient.

More specifically, the thin film can undergo: (i) polymorphic transition of active ingredients; (ii) hydration of polymers of the oral thin film; and (iii) decomposition and oxidation of the active ingredient by photolytic or hydrolytic degradation.

More specifically, the self-emulsions in the systems and methods described herein, can increase properties 1 and 2. Based on properties 1 and 2, the systems and methods described herein, can: (i) provide effective protection to the active ingredient from damage during manufacturing processes; and (ii) impart reversibility during gelation phenomena in self-emulsifying oral thin films.

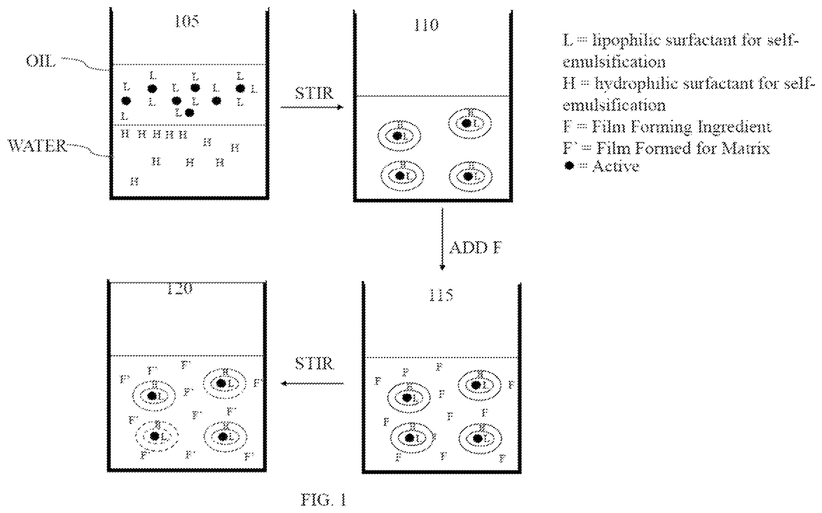

With respect to property 1, the emulsion as shown in FIG. 1 and matrix can stabilize the oral thin film containing the active ingredient by blocking water, light, and heat from the active ingredient. In beaker 105, there are two immiscible layers—the aqueous layer which has dissolved hydrophilic surfactants (H) on the bottom and the oil layer which has dissolved lipophilic surfactants (L) and active ingredients.

Upon vigorous mixing of the contents in beaker 105, the lipophilic surfactants (L) and hydrophilic surfactants (H) make an emulsion which can surround the active ingredient, as depicted in beaker 110. By virtue of the active ingredient being lipophilic and having high solubility in organic solvents and low solubility in water, the active ingredient is proximal to L (i.e., L is attracted to the active ingredient) and distal to H (i.e., H is repulsed by the active ingredient) in beaker 110. Film forming ingredients (F), as listed in the tables below, are added to beaker 110 and thus resulting in beaker 115.

If light or heat is added, certain active ingredients, such as Vitamin D3, may undergo cycloaddition reactions with some of the film forming ingredients. The addition of light and heat can excite electrons in pi-systems of dienes, such as those found in Vitamin D3, and undergo Diels-Alder reactions with an electron deficient alkene of the sorbates, which is one of the film forming ingredients (F). The resulting Diels-Alder adduct is an undesirable side product. This side product is difficult to remove during the manufacturing process, while reducing the overall yield of the active ingredient to be administered and release the individual consuming the OTF.

The emulsion where L is proximal to the active ingredient and H is distal to the active ingredient surrounds the active ingredient and thus is a chemo-physical barrier against light, water, heat, oxidation, and other degradative processes. After mixing the contents of beaker 115, F can be converted to the film for constructing the matrix (F′) in the initial stages of slurry formation, as depicted in beaker 120. The emulsion begins to disintegrate, as depicted by the dotted line, to a precursor to be embedded into F′.

For blocking water from the active ingredient, the active ingredient is surrounded by a lipophilic surfactant such that interactions with water and the active ingredient are reduced or eliminated (i.e., reduced hygroscopicity). For blocking light from the active ingredient, the self-emulsions are an additional layer, which is a physical barrier which can impede the entry of light. For blocking heat from the active ingredient, the gelation phenomena (i.e., gel matrix) dissipates heat and thereby reducing heat interactions with the active ingredient (i.e., reduced thermal degradation of the active ingredient).

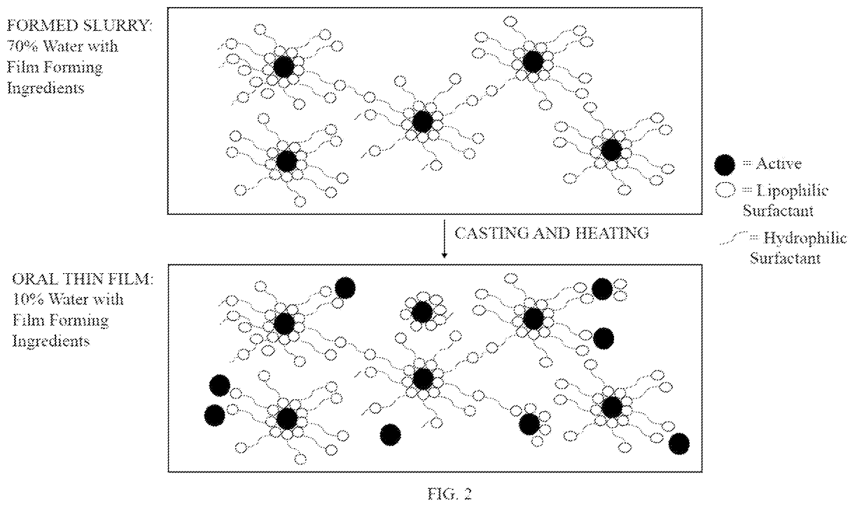

With respect to property 2, the compositions of the oral thin film which self-emulsify can improve bioavailability of the active ingredients. As depicted in FIG. 2, the top and bottom depictions are absent of the emulsions of FIG. 1 after completing the mixing and heating. The top depiction in FIG. 2 is a slurry that is 70% water (by weight) with film forming ingredients converted to the matrix. The active ingredients are surrounded by micelles where the active ingredient is dissolved in oil. The head of the micelles are lipophilic surfactants (L) in direct contact with the oil and the tail of the micelles are hydrophilic surfactants (H) are in direct contact with water. After heating the slurry in an oven, the bottom depiction in FIG. 2 a dried OTF is formed. The amount of water in the bottom depiction in FIG. 2 is reduced to 10% water (by weight). The concentrations of active ingredients and micelles are increased in the bottom depiction in FIG. 2 in comparison to the top depiction in FIG. 2. This aides in increasing the bioavailability of the active ingredients prior to and during administration of the OTF.

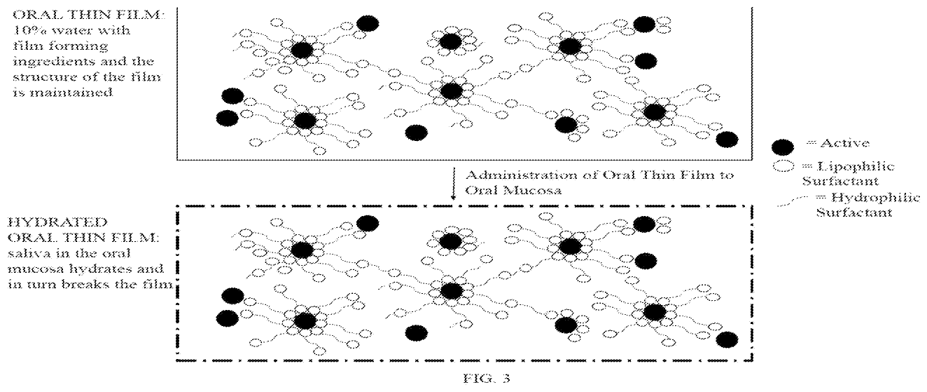

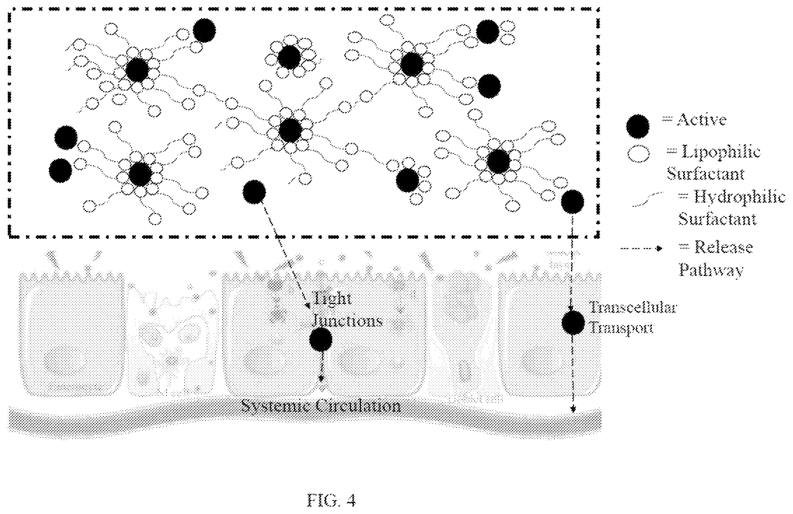

With further respect to property 2, upon administering this oral thin film, which becomes hydrated by the oral mucosa; the oral thin film starts to disintegrate into the oral mucosa, as depicted in FIG. 3. For example, the surfactant system of the OTF (e.g., lipophilic and hydrophilic surfactants) can self-emulsify in the mouth and facilitate the release of the active ingredient for systemic circulation by transcellular transport (lipophilic surfactants) and/or paracellular transport (hydrophilic surfactants), as depicted in FIG. 4.

With further respect to property 2 and as depicted in FIG. 4, transcellular transport refers to the pathway of a substance through the epithelial cell by transcytosis. Transcytosis is a process by which particles are taken up by cells, depending on various physicochemical properties of particles, such as lipophilicity. Self-emulsifying oral thin films of the systems and methods described herein, can modify the lipophilicity of the active ingredient and facilitate movement of the active ingredient through transcellular diffusion pathways. As depicted in FIG. 4, the oral thin film disintegrates and therefore releases the active ingredient for transcellular diffusion. The transcellular diffusion involves the movement of active ingredient based on a diffusion gradient moving from an area of high concentration to an area of low concentration.

With further respect to property 2 and as depicted in FIG. 4, paracellular transport refers to the transfer of substances, such as active ingredients or food ingredients, across an epithelium by passing through the intercellular space in between the cells. The intercellular space between the cells can be minimal and thus rendering the intercellular space as a tight junction. The transfer of substances can require modulation of the tight junction. The components of self-emulsifying thin film can modulate the tight junction. More specifically, a first fatty acid chain of the surfactant system can allow adhesion of the active ingredient at the epithelial cell surface for longer times than observed without the surfactant system. Additionally, a second fatty acid chain of the surfactant system can inhibit the mechanism for contracting the intercellular space. The adhesion and inhibited mechanism for contracting the intercellular space can loosen the tight junction to yield a less tight junction to facilitate penetration of the epithelium by the active ingredient. The loosening mechanism is reversible and thus allows the intercellular cell space to revert to the tight junction. As depicted in FIG. 4, the oral thin film disintegrates and therefore releases the active ingredient for modifying the tight junctions.
The systems and methods described herein, are a platform technology that can be integrated to multiple products. However, there may be small differences in the ingredients and/or order of addition without departing from the scope of the claims and maintaining features 1-3. Applications of the platform technology are transdermal/topical patches, creams, balms, semi-solid products, and processes that do not hold a substantial amount of water. A substantial amount of water is an amount of water by weight percent that can have adverse effects of the efficacy of the thin film.

### EXEMPLARY EMBODIMENTS AND COMBINATIONS

| | | Amount | Amount | Amount |
|---|---|---|---|---|
| Ingredients | Function(s) | (mg) | (range, mg) | (wt. %) |
| Lipophilic active ingredient or | API | 1.25 or 50 | 0.25-150 | 0.5% to 60% |
| Hydrophilic active ingredient | | | | |
| Glyceryl Monocaprylate | Lipophilic Self- | 50 | 1-100 | 0.5% to 40% |
| | Emulsifying Surfactant/ | | | |
| | Solvent for Lipophilic | | | |
| | Active ingredient | | | |
| Propylene Glycol | Lipophilic Self- | 50 | 1-100 | 0.5% to 40% |
| Monocaprylate | Emulsifying Surfactant/ | | | |
| | Solvent for Lipophilic | | | |
| | Active ingredient | | | |
| Glyceryl Monooleate | Lipophilic Self- | 50 | 1-100 | 0.5% to 40% |
| | Emulsifying Surfactant/ | | | |
| | Solvent for Lipophilic | | | |
| | Active ingredient | | | |
| Propylene Glycol monolaurate | Lipophilic Self- | 50 | 1-100 | 0.5% to 40% |
| | Emulsifying Surfactant/ | | | |
| | Solvent for Lipophilic | | | |
| | Active ingredient | | | |
| Glyceryl Caprylate/Caprate | Lipophilic Self- | 50 | 1-100 | 0.5% to 40% |
| | Emulsifying Surfactant/ | | | |
| | Solvent for Lipophilic | | | |
| | Active ingredient | | | |
| Glyceryl monolinoleate | Lipophilic Self- | 50 | 1-100 | 0.5% to 40% |
| | Emulsifying Surfactant/ | | | |
| | Solvent for Lipophilic | | | |
| | Active ingredient | | | |
| Sorbitan Mmonooleate | Lipophilic Self- | 50 | 1-100 | 0.5% to 40% |
| (Span 80) | Emulsifying Surfactant/ | | | |
| | Solvent for Lipophilic | | | |
| | Active ingredient | | | |
| Medium Chain Triglycerides | Solvent for Lipophilic | 50 | 1-100 | 0.5% to 40% |
| Oil | Active ingredient | | | |
| Coconut Oil | Solvent for Lipophilic | 50 | 1-100 | 0.5% to 40% |
| | Active ingredient | | | |
| Corn Oil | Solvent for Lipophilic | 50 | 1-100 | 0.5% to 40% |
| | Active ingredient | | | |
| Olive Oil | Solvent for Lipophilic | 50 | 1-100 | 0.5% to 40% |
| | Active ingredient | | | |
| Palm Oil | Solvent for Lipophilic | 50 | 1-100 | 0.5% to 40% |
| | Active ingredient | | | |
| Canola Oil | Solvent for Lipophilic | 50 | 1-100 | 0.5% to 40% |
| | Active ingredient | | | |
| Safflower Oil | Solvent for Lipophilic | 50 | 1-100 | 0.5% to 40% |
| | Active ingredient | | | |
| Sesame Oil | Solvent for Lipophilic | 50 | 1-100 | 0.5% to 40% |
| | Active ingredient | | | |
| Polyoxyl castor oil | Hydrophilic Surfactant | 50 | 1-100 | 0.5% to 40% |
| Polyethylene-polypropylene | Hydrophilic Surfactant | 50 | 1-100 | 0.5% to 40% |
| glycol | | | | |
| Polyoxyethylene sorbitan | Hydrophilic Surfactant | 50 | 1-100 | 0.5% to 40% |
| monolaurate (Tween 20) | | | | |
| Tween 80 | Hydrophilic Surfactant | 50 | 1-100 | 0.5% to 40% |
| polyoxyethylenesorbitan | Hydrophilic Surfactant | 50 | 1-100 | 0.5% to 40% |
| monostearate (Tween 60) | | | | |
| Diethylene Glycol Monoethyl | Co-Solvent | 20 | 1-100 | 0.5% to 40% |
| ether | | | | |
| Caprylocapryol Polyoxyl-8 | Co-Solvent | 20 | 1-100 | 0.5% to 40% |
| glycerides | | | | |
| Propylene Glycol | Plasticizer | 10 | 2-80 | 0.5% to 20% |
| Glycerin | Plasticizer | 10 | 2-80 | 0.5% to 20% |
| Pullulan | Film Forming Polymer | 30 | 10-100 | 10% to 60% |
| *Chitosan | Film Former | 20 | 1 to 50 | 1% to 60% |
| Pectin | Film Former | 20 | 1 to 50 | 1% to 60% |
| *Carrageenan | Film Forming Polymer | 30 | 10-100 | 10% to 60% |
| *HPMC | Film Former | 10 | 1 to 50 | 1% to 60% |
| *HPC | Film Former | 30 | 1 to 50 | 1% to 60% |
| Modified Corn Starch | Film Forming Polymer | 70 | 10-200 | 10% to 60% |
| Glyceryl Dibehenate | Lipophilic Self | 50 | 1-100 | 0.5% to 40% |
| | Emulsifying Surfactant/ | | | |
| | Solvent for lipophilic | | | |
| | Active ingredient | | | |
| Propylene Glycol Dilaurate | Lipophilic Self | 50 | 1-100 | 0.5% to 40% |
| | Emulsifying Surfactant/ | | | |
| | Solvent for Lipophilic | | | |
| | Active ingredient | | | |
| Glyceryl | Lipophilic Self | 50 | 1-100 | 0.5% to 40% |
| Tricaprylate/Tricaprate | Emulsifying Surfactant/ | | | |
| | Solvent for Lipophilic | | | |
| | Active ingredient | | | |
| Glycerol | Lipophilic Self | 50 | 1-100 | 0.5% to 40% |
| Tricaprylate/Caprate | Emulsifying Surfactant/ | | | |
| | Solvent for Lipophilic | | | |
| | Active ingredient | | | |
| Glyceryl Tricaprylate | Lipophilic Self | 50 | 1-100 | 0.5% to 40% |
| | Emulsifying Surfactant/ | | | |
| | Solvent for Lipophilic | | | |
| | Active ingredient | | | |
| Decaglycerol Mono and Di | Lipophilic Self | 50 | 1-100 | 0.5% to 40% |
| Oleate | Emulsifying Surfactant/ | | | |
| | Solvent for Lipophilic | | | |
| | Active ingredient | | | |
| Oleoyl Macrogolglycerides | Lipophilic Self | 50 | 1-100 | 0.5% to 40% |
| | Emulsifying Surfactant/ | | | |
| | Solvent for Lipophilic | | | |
| | Active ingredient | | | |
| Lauroyl Macrogolglycerides | Lipophilic Self | 50 | 1-100 | 0.5% to 40% |
| | Emulsifying Surfactant/ | | | |
| | Solvent for Lipophilic | | | |
| | Active ingredient | | | |
| Stearoyl Macrogolglycerides | Lipophilic Self | 50 | 1-100 | 0.5% to 40% |
| or Stearoyl | Emulsifying Surfactant/ | | | |
| Polyoxylglycerides | Solvent for Lipophilic | | | |
| | Active ingredient | | | |
| Polyoxyethylene | Lipophilic Self | 50 | 1-100 | 0.5% to 40% |
| Caprylic/Capric Glycerides | Emulsifying Surfactant/ | | | |
| | Solvent for Lipophilic | | | |
| | Active ingredient | | | |
| Cetyal Alcohol | Solvent for Lipophilic | 50 | 1-100 | 0.5% to 40% |
| | Active ingredient | | | |
| Steryl Alcohol | Solvent for Lipophilic | 50 | 1-100 | 0.5% to 40% |
| | Active ingredient | | | |
| Cetostryl Alcohol | Solvent for Lipophilic | 50 | 1-100 | 0.5% to 40% |
| | Active ingredient | | | |
| Oleyl Alcohols | Solvent for Lipophilic | 50 | 1-100 | 0.5% to 40% |
| | Active ingredient | | | |
| Brij | Lipophilic Self | 50 | 1-100 | 0.5% to 40% |
| | Emulsifying Surfactant/ | | | |
| | Solvent for Lipophilic | | | |
| | Active ingredient | | | |
| Decyl Glucoside | Hydrophilic Surfactant | 50 | 1-100 | 0.5% to 40% |
| Lauryl Glucoside | Hydrophilic Surfactant | 50 | 1-100 | 0.5% to 40% |
| Octyl Glucoside | Hydrophilic Surfactant | 50 | 1-100 | 0.5% to 40% |
| Triton X-100 | Hydrophilic Surfactant | 50 | 1-100 | 0.5% to 40% |
| Nonoxynol 9 | Hydrophilic Surfactant | 50 | 1-100 | 0.5% to 40% |
| Glyceryl Laurate | Lipophilic Self | 50 | 1-100 | 0.5% to 40% |
| | Emulsifying Surfactant/ | | | |
| | Solvent for Lipophilic | | | |
| | Active ingredient | | | |
| Sodium Lauryl Sulfate | Hydrophilic Surfactant | 50 | 1-100 | 0.5% to 40% |
| Potassium Lauryl Sulfate | Hydrophilic Surfactant | 50 | 1-100 | 0.5% to 40% |
| Phospholipids | Lipophilic Self | 50 | 1-100 | 0.5% to 40% |
| | Emulsifying Surfactant/ | | | |
| | Solvent for Lipophilic | | | |
| | Active ingredient | | | |
| n-dodecyl phosphocholine | Lipophilic Self | 50 | 1-100 | 0.5% to 40% |
| | Emulsifying Surfactant/ | | | |
| | Solvent for Lipophilic | | | |
| | Active ingredient | | | |
| Cholesteryl esters | Lipophilic Self | 50 | 1-100 | 0.5% to 40% |
| | Emulsifying Surfactant/ | | | |
| | Solvent for lipophilic | | | |
| | Active ingredient | | | |
| BHT or other Antioxidant | Antioxidants | 0.15 | 0.1-10 | 0.1% to 10% |
| agents | | | | |
| Potassium sorbate or other | Antimicrobial agents | 0.1 | 0.1 to 0.5 | 0.05% to 0.5% |
| antimicrobial agents | | | | |
| Menthol or other Flavors | Flavors | 0.1 | 0.1 to 20 | 0.05% to 20% |
| Red 40, Blue 1, Yellow 5 | Coloring Agents | 0.005 | 0.001 to 0.1 | 0.005% to 0.5% |
| Sucralose or other sweeteners | Sweeteners | 0.1 | 0.1 to 10 | 0.05% to 10% |

*Specific Grades of Film formers preferred towards film formulation for buccal administration.

### Example 1

| | | Amount | |
|---|---|---|---|
| | | (mg)/ | % W/W |
| Material | Function | Strip | Dry |
| Vitamin D3 | Active ingredient | 1.570 | 1.35 |
| Tween 20 | Hydrophilic Surfactant | 3.650 | 3.13 |
| Span 80 | Lipophilic Surfactant | 1.100 | 0.94 |
| MCT Oil | Solvent for API | 3.540 | 3.04 |
| Butylated | Antioxidant | 0.150 | 0.13 |
| Hydroxytoluene (BHT) | | | |
| Flavors | | | 0.00 |
| Sucralose USP/NF | Sweetener | 1.573 | 1.35 |
| Nat & Art Mixed | Flavor | 5.755 | 4.94 |
| Berry Flavor | | | |
| Mountain Berry | Flavor | 7.289 | 6.26 |
| Film Forming System | | | 0.00 |
| Modified Food Starch | Film Former Polymer | 56.23 | 48.28 |
| Pullulan | Film Former Polymer | 20.030 | 17.20 |
| Glycerin 99.7% USP | Plasticizer | 15.550 | 13.35 |
| Potassium Sorbate | Antimicrobial | 0.1 | 0.08 |
| Red 40 | Coloring Agent | 0.015 | 0.01 |
| Purified Water* | N/A | 271.71* | N/A |
| Total | | 116.452 | 100.00 |

Note:
*Purified water is used only for processing. During Film making process, water is removed during the drying processes. Only 5-15% moisture remains in the film. Typically, a total of 70% water is used, while the rest of the ingredients amounts to 30% of the dried weight.

Experimental procedure
1) Dissolve Vitamin D3 and BHT in MCT oil by warming to 40° C.±5° C. and thereby yielding Mix 1.
2) Add Span 80 to Mix 1 and thereby yielding Mix 2.
3) Add Tween 20 with purified water (20% of the 70% as prescribed above) to Mix 2 and thereby yielding Mix 3, where the temperature of the water is 40° C.±5° C.
4) Agitate Mix 3 via mechanical agitation.
5) Add remaining water to Mix 3, where the temperature of the water is 80° C.±5° C.
6) Add modified food starch, pullulan, glycerin, potassium sorbate, Red 40, sucralose, and berry flavors Mix 3 and thereby yielding Mix 4.
7) Agitate Mix 4 until Mix 4 reaches a homogenous state and thereby yielding a slurry.
8) Run the slurry for film casting processes and thereby yielding a thin film.
9) After casting, dry the thin film in drying oven for no more than 15 minutes, or until dried at 160° F. to 180° F. Measure the thickness of the thin film, where the specification of the thin film is 0.12 mm to 0.20 mm. This can be adjusted during the casting process.
10) After drying, cut the thin film such that there are 22 mm by 36 mm strips. This can be adjusted as per Dose.

### Example 2

| | | Amount | |
|---|---|---|---|
| | | (mg)/ | % W/W |
| Material | Function | Strip | Dry |
| Vitamin D3 | Active ingredient | 1.570 | 1.22 |
| Kolliphor RH40 | Hydrophilic Surfactant | 16.00 | 12.41 |
| Span 80 | Lipophilic Surfactant | 1.100 | 0.85 |
| MCT Oil | Solvent for API | 3.540 | 2.75 |
| Butylated | Antioxidant | 0.150 | 0.12 |
| Hydroxytoluene (BHT) | | | |
| Flavors | | | 0.00 |
| Sucralose USP/NF | Sweetener | 1.573 | 1.22 |
| Nat & Art Mixed | Flavor | 5.755 | 4.46 |
| Berry Flavor | | | |
| Mountain Berry | Flavor | 7.289 | 5.65 |
| Film Forming System | | | 0.00 |
| Modified Food Starch | Film Former Polymer | 56.23 | 43.62 |
| Pullulan | Film Former Polymer | 20.030 | 15.54 |
| Glycerin 99.7% USP | Plasticizer | 15.550 | 12.06 |
| Potassium Sorbate | Antimicrobial | 0.1 | 0.08 |
| Red 40 | Coloring Agent | 0.015 | 0.01 |
| Purified Water* | N/A | 271.71* | N/A |
| Total | | 128.90 | 100.00 |

Note:
*Purified water is used only for processing. During Film making process, water is removed during the drying processes. Only 5-15% moisture remains in the film. Typically, a total of 70% water is used, while the rest of the ingredients amounts to 30% of the dried weight.

### Experimental Procedure

1) Dissolve Vitamin D3 and BHT in MCT oil by warming MCT oil to 40° C.±5° C. and thereby yielding Mix 1.
2) Add Span 80 to Mix 1 and thereby yielding Mix 2.
3) Add Kolliphor RH40 with Purified water (20% of the 70%) and thereby yielding Mix 3, where the temperature of the water is 40° C. f 5° C.
4) Agitate Mix 3 via mechanical agitation.
5) Add remaining water to Mix 3, where the temperature of the water is 80° C.±5° C.
6) Add Modified Food starch, Pullulan, Glycerin, Potassium sorbate, Red 40, Sucralose and Berry Flavors to Mix 3 and thereby yielding Mix4.
7) Agitate Mix 4 until Mix 4 reaches a homogenous state and thereby yielding a slurry.
8) Run the slurry for film casting processes and thereby yielding a thin film.
9) After casting, dry the thin film in drying oven for no more than 15 minutes, or until dried at 160° F. to 180° F. Measure the thickness of the thin film, where the specification of the thin film is 0.12 mm to 0.20 mm. (This can be adjusted during the casting process.)
10) After drying, cut the thin film such that there are 22 mm by 36 mm strips. (This can be adjusted as per Dose)

### Example 3

| | | Amount | |
|---|---|---|---|
| | | (mg)/ | % W/W |
| Material | Function | Strip | Dry |
| Vitamin D3 | Active ingredient | 1.570 | 1.22 |
| Poloxamer 407 | Hydrophilic Surfactant | 16.00 | 12.41 |
| Span 80 | Lipophilic Surfactant | 1.100 | 0.85 |
| MCT Oil | Solvent for API | 3.540 | 2.75 |
| Butylated | Antioxidant | 0.150 | 0.12 |
| Hydroxytoluene (BHT) | | | |
| Flavors | | | 0.00 |
| Sucralose USP/NF | Sweetener | 1.573 | 1.22 |
| Nat & Art Mixed | Flavor | 5.755 | 4.46 |
| Berry Flavor | | | |
| Mountain Berry | Flavor | 7.289 | 5.65 |
| Film Forming System | | | 0.00 |
| Modified Food Starch | Film Former Polymer | 56.23 | 43.62 |
| Pullulan | Film Former Polymer | 20.030 | 15.54 |
| Glycerin 99.7% USP | Plasticizer | 15.550 | 12.06 |
| Potassium Sorbate | Antimicrobial | 0.1 | 0.08 |
| Red 40 | Coloring Agent | 0.015 | 0.01 |
| Purified Water* | N/A | 271.71* | N/A |
| Total | | 128.90 | 100.00 |

Note:
*Purified water is used only for processing. During Film making process, water is removed during the drying processes. Only 5-15% moisture remains in the film. Typically, a total of 70% water is used, while the rest of the ingredients amounts to 30% of the dried weight.

### Experimental Procedure

1) Dissolve Vitamin D3 and BHT into MCT oil by warming the MCT oil to 40° C.±5° C. and thereby yielding Mix 1.
2) Add Span 80 to Mix 1 and thereby yielding Mix 2.
3) Add Poloxamer 407 with Purified water (20% of the 70% as prescribed above) to Mix 2 and thereby yielding Mix 3, where the temperature of the water is 40° C.±5° C.
4) Agitate Mix 3 via mechanical agitation.
5) Add remaining water to Mix 3, where the temperature of the water is 80° C.±5° C.
6) Add Modified Food starch, Pullulan, Glycerin, Potassium sorbate, Red 40, Sucralose and Berry Flavors to Mix 3 and thereby yielding Mix 4.
7) Agitate Mix 4 until Mix 4 reaches a homogenous mixture and thereby yielding a slurry.
8) Run the slurry for film casting processes and thereby yielding a thin film.
9) After casting, dry the thin film in drying oven for no more than 15 minutes, or until dried at 160° F. to 180° F. Measure the thickness of the thin film, where the specification of the thin film is 0.12 mm to 0.20 mm. (This can be adjusted during the casting process.)
10) After drying, cut the thin film such that there are 22 mm by 36 mm strips. (This can be adjusted as per Dose.)

### Example 4

| | | Amount | |
|---|---|---|---|
| | | (mg)/ | % W/W |
| Material | Function | Strip | Dry |
| CBD Isolate | Active ingredient | 50 | 21.66 |
| Tween 20 | Hydrophilic Surfactant | 25 | 10.83 |
| Span 80 | Lipophilic Surfactant | 5 | 2.17 |
| Propylene Glycol | Lipophilic Surfactant/ | 50 | 21.66 |
| Monocaprylate | Solvent for API | | |
| Flavors | | | |
| Sucralose USP/NF | Sweetener | 1.573 | 0.68 |
| Mint Flavor | Flavor | 7.289 | 3.16 |
| Film Forming System | | | |
| Modified Food Starch | Film Former Polymer | 56.23 | 24.36 |
| Pullulan | Film Former Polymer | 20.03 | 8.68 |
| Glycerin 99.7% USP | Plasticizer | 15.55 | 6.74 |
| Potassium Sorbate | Antimicrobial | 0.1 | 0.04 |
| Yellow 5 | Coloring Agent | 0.03 | 0.01 |
| Red 40 | Coloring Agent | 0.015 | 0.01 |
| Purified Water* | N/A | 538.58* | |
| | | 230.82 | 100.00 |

Note:
*Purified water is used only for processing. During the film making process, water is removed during the drying processes. Only 5-15% moisture remains in the film. Typically, a total of 70% water is used, while the rest of the ingredients amounts to 30% of the dried weight.

### Experimental Procedure

1) Dissolve CBD isolate into propylene glycol monocaprylate by warming propylene glycol monocaprylate at 55° C. f 5° C. and thereby yielding Mix 1.
2) Add Span 80 to Mix 1 and thereby yielding Mix 2.
3) Add Tween 20 with Purified water (20% of the 70% of the prescribed amount) to Mix 2 and thereby yielding Mix 3, where the temperature of the water is 55° C.±5° C.
4) Agitate Mix 3 via mechanical agitation.
5) Add remaining water to Mix 3, where the temperature of the water is 80-C f 5 CC.
6) Add Modified Food starch, Pullulan, Glycerin, Potassium sorbate, Red 40, Sucralose and Berry Flavors to Mix 3 and thereby yielding Mix 4.
7) Agitate Mix 4 until Mix 4 reaches a homogenous mixture and thereby yielding a slurry.
8) Run the slurry for film casting processes and thereby yielding a thin film.
9) After casting, dry the thin film in drying oven for no more than 15 minutes, or until dried at 160° F. to 180° F. Measure the thickness of the thin film, where the specification of the thin film is 0.12 mm to 0.20 mm. (This can be adjusted dung the casting process.)
10) After drying, cut the thin film such that there are 22 mm by 36 mm strips. (This can be adjusted as per Dose.)

### Example 5

| | | Amount | |
|---|---|---|---|
| | | (mg)/ | % W/W |
| Material | Function | Strip | Dry |
| CBD Isolate | Active ingredient | 50 | 21.66 |
| Tween 20 | Hydrophilic Surfactant | 25 | 10.83 |
| Span 80 | Lipophilic Surfactant | 5 | 2.17 |
| Propylene Glycol | Lipophilic Surfactant/ | 50 | 21.66 |
| Monolaurate | Solvent for API | | |
| Flavors | | | |
| Sucralose USP/NF | Sweetener | 1.573 | 0.68 |
| Mint Flavor | Flavor | 7.289 | 3.16 |
| Film Forming System | | | |
| Modified Food Starch | Film Former Polymer | 50.00 | 20.95 |
| Chitosan | Film Former Polymer | 6.23 | 2.70 |
| Pectin | Film Former Polymer | 20.03 | 8.68 |
| Glycerin 99.7% USP | Plasticizer | 15.55 | 6.74 |
| Potassium Sorbate | Antimicrobial | 0.1 | 0.04 |
| Yellow 5 | Coloring Agent | 0.03 | 0.01 |
| Red 40 | Coloring Agent | 0.015 | 0.01 |
| Purified Water* | N/A | 538.58* | |
| | | 230.82 | 100.00 |

Note:
*Purified water is used only for processing. During the film making process, water is removed during the drying processes. Only 5-15% moisture remains in the film. Typically, a total of 70% water is used, while the rest of the ingredients amounts to 30% of the dried weight.

### Experimental Procedure

1) Dissolve CBD isolate into propylene glycol monocaprylate by warming propylene glycol monocaprylate at 55° C. f VOC and thereby yielding Mix 1.
2) Add Span 80 to Mix 1 and thereby yielding Mix 2.
3) Add Tween 20 with Purified water (20% of the 70% of the prescribed amount) to Mix 2 and thereby yielding Mix 3, where the temperature of the water is 55° C.±5° C.
4) Agitate Mix 3 via mechanical agitation.
5) Add remaining water to Mix 3, where the temperature of the water is 80° C.±5° C.
6) Add Modified Food starch, Pectin, Chitosan, Glycerin, Potassium sorbate, Red 40, Sucralose and Berry Flavors to Mix 3 and thereby yielding Mix 4.
7) Agitate Mix 4 until Mix 4 reaches a homogenous mixture and thereby yielding a slurry.
8) Run the slurry for film casting processes and thereby yielding a thin film.
9) After casting, dry the thin film in drying oven for no more than 15 minutes, or until dried at 160° F. to 180° F. Measure the thickness of the thin film, where the specification of the thin film is 0.12 mm to 0.20 mm. (This can be adjusted during the casting process.)
10) After drying, cut the thin film such that there are 22 mm by 36 mm strips. (This can be adjusted as per Dose.)

### Stability Study and Data

| | | Time Points And % Assay of CBD | | | | | |
|---|---|---|---|---|---|---|---|
| | Stability | T0 | T 2 | T1 | T2 | T3 | T9 |
| Drug | Condition | Week | Weeks | Month | Months | Months | Months |
| CBD | 25 C./ | 108% | 109% | 109% | 113% | 120% | |
| | 60% RH | | | | | | |
| | 40 C./ | | 108% | 106% | 113% | 118% | 96% |
| | 75% RH | | | | | | |

1) The primary application of the technology is to provide better stability and increase bioavailability of the active ingredient
2) Composition of Self emulsifying delivery can be incorporated into OTF and other application (i.e. semi-solids) such as patches.

### ALTERNATIVE EMBODIMENTS

### Sandimmune® (Cyclosporine A/I)

Indicated for the organ rejection prophylaxis in allogenic transplants of kidney, liver, and heart Corn oil, linoleoylmacrogol glycerides, and sorbitol

### Neora® (Cyclosporine)

Systemic immunosuppressant
Corn oil-mono-di-triglycerides, polyoxyl 40 hydrogenated castor oil NF, DL-α tocopherol USP

### Gengraf® (Cyclosporine A/III)

Systemic immunosuppressant
Polyethylene glycol NF, polyoxyl 35 castor oil NF, polysorbate 80 NF, propylene glycol USP, sorbitan monooleate NF, titanium dioxide

### Norvir® (Ritonavir)

Combination with other antiretroviral agents for the treatment of HIV-1 infection
Butylated hydroxytoluene, ethanol, oleic acid, polyoxyl 35, and castor oil

### Fortovase® (Saquinavir)

Inhibitor of the human immunodeficiency virus (HIV) protease
Medium chain mono and diglycerides, povidone, and dl-alpha-tocopherol

### Agenerase® (Amprenavir)

Inhibitor of the human immunodeficiency virus (HIV) protease
d-alpha tocopherol PEG 1000 succinate (TPGS), PEG 400, and propylene glycol

### Depakene® (Valproic Acid)

Monotherapy and adjunctive therapy in the treatment of patients with complex partial seizures that occur either in isolation or in association with other types of seizures
Corn oil, glycerin, methylparaben, and propylparaben

### Rocaltrol® (Calcitriol)

Management of secondary hyperparathyroidism and management of hypocalcemia
Triglyceride of coconut oil

### Targretin® (Bexarotene)

Treatment of cutaneous manifestations of cutaneous T-cell lymphoma in patients who are refractory to at least one prior systemic therapy
Polyethylene glycol 400, NF, Polysorbate 20, NF, povidone, USP, and butylated hydroxyanisole, NF

### Vesanoid® (Tretinoin)

Retinoid that induces maturation of acute promyelocytic leukemia (APL)
Beeswax, butylated hydroxyanisole, edetate disodium, hydrogenated soybean oil flakes, hydrogenated vegetable oils, and soybean oil

### Accutane® (Isotretinoin)

Severe recalcitrant nodular acne
Beeswax, butylated hydroxyanisole, edetate disodium, hydrogenated soybean oil flakes, hydrogenated vegetable oil, and soybean oil

### Aptivus® (Tipranavir)

Combination antiretroviral treatment of HIV-1
Dehydrated alcohol (7% w/w or 0.1 g per capsule), polyoxyl 35 castor oil, propylene glycol, mono/diglycerides of caprylic/capric acid

### Experimental Data

### Pharmacokinetic Study and Data

Open label randomized crossover and balanced study of single dose administration, the described delivery system has improved bioavailability of CBD when compared to administration of commercially available soft gel. The study was conducted with healthy adults under fasting condition with equal representation of gender (8/6 male female).

1-57. (canceled)

58. A method of forming an oral dissolvable film, the method comprising:
(a) dissolving an active pharmaceutical ingredient in a first solvent-system to form a first mixture, wherein:
(i) when the active pharmaceutical ingredient is lipophilic or hydrophobic, dissolving the active pharmaceutical ingredient in a lipophilic or hydrophobic solvent, in a lipophilic or hydrophobic surfactant, or combination thereof; or
(ii) when the active pharmaceutical ingredient is hydrophilic or lipophobic, dissolving the active pharmaceutical ingredient in a hydrophilic or lipophobic solvent, in a hydrophilic or lipophobic surfactant, or combination thereof;
(b) contacting the first mixture and a lipophilic or hydrophobic surfactant to form a second mixture;
(c) contacting the second mixture with water and a hydrophilic or lipophobic surfactant to form a third mixture;
(d) contacting the third mixture with film forming ingredient to form a slurry; and
(e) casting the slurry on a substrate and curing to form the oral dissolvable film.

59. The method of claim 58, wherein the film forming ingredient comprises at least one of mucoadhesive polymer, plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, solvent, permeation enhancer, and preservative.

60. The method of claim 58, wherein the lipophilic or hydrophobic solvent comprises an oil.

61. The method of claim 58, wherein the hydrophilic or lipophobic solvent comprises an aqueous liquid.

62. The method of claim 58, wherein the curing is carried out in a hot air oven at an air temperature of between about 38° C. to about 110° C.

63. The method of claim 58, wherein the curing is carried out in a hot air oven at an air temperature of between about 45° C. to about 80° C.

64. The method of claim 58, wherein the curing is carried out in a hot air oven (at an air temperature of 50° C.-70° C.).

65. The method of claim 58, wherein the curing is carried out at a speed of between about 0.8 feet/min to about 2.5 feet/min.

66. The method of claim 58, wherein the curing is carried out at a speed of between about 0.8 feet/min to about 1.0 feet/min.

67. The method of claim 58, wherein the curing is carried out at a speed of between about 2.0 feet/min to about 2.5 feet/min.

68. The method of claim 58, wherein the oral dissolvable film comprises:
(a) hydrophilic active pharmaceutical ingredient;
(b) water carrier for the hydrophilic active pharmaceutical ingredient;
(c) hydrophilic surfactant for the hydrophilic active pharmaceutical ingredient;
(d) one or more co-surfactants;
(e) one or more self-emulsifying surfactants;
(f) film matrix; and
(g) water.

69. The method of claim 58, wherein the oral dissolvable film is configured to self-emulsify within 20 seconds upon contact with an oral mucosal surface of a subject.

70. The method of claim 58, wherein the oral dissolvable film is configured to form an oil-in-water (O/W) emulsion within 20 seconds upon contact with an oral mucosal surface of a subject.

71. The method of claim 58, wherein the oral dissolvable film is configured to form an oil-in-water (O/W) emulsion having an average droplet size of 0.1 microns to 120 microns within 20 seconds upon contact with an oral mucosal surface of a subject.

72. The method of claim 58, wherein the oral dissolvable film is configured to form an oil-in-water (O/W) emulsion having an average droplet size of
d(10): 0.5-10 micron,
d(50): 1-20 micron, and
d(90): 15-100 micron
within 20 seconds upon contact with an oral mucosal surface of a subject.

73. The method of claim 58, wherein the oral dissolvable film is suitable for oral administration (PO), buccal administration, sublingual administration, or mucosal administration.

74. The method of claim 58, wherein the oral dissolvable film has a moisture content of 3-13 wt. %.

75. The method of claim 58, wherein the oral dissolvable film is configured to disintegrate within 15 minutes upon buccal administration to a subject.

76. The method of claim 58, wherein the oral dissolvable film is configured to disintegrate within 30 seconds upon oral (PO) administration to a subject.

77. The method of claim 58, wherein the oral dissolvable film is configured for in vitro disintegration (USP<701> In-vitro Disintegration method) within 30 seconds.

78. The method of claim 58, wherein the oral dissolvable film exhibits at least one pharmacokinetic parameter selected from, (i) Tmax of between about 45 min to about 120 min, (ii) Cmax of at least 3.5 ng/ml, and (iii) AUC₀₋ₜ of at least 13 ng/hr/ml.

79. The method of claim 58, wherein the oral dissolvable film exhibits at least one pharmacokinetic parameter selected from, (i) Tmax of 1.5 hr, (ii) Cmax of 4.4 ng/ml, and (iii) AUC₀₋ₜ of 13.5 ng/hr/ml.

80. The method of claim 58, wherein the oral dissolvable film exhibits an in vivo dissolution time of no more than 20 minutes.

81. The method of claim 58, wherein the oral dissolvable film exhibits a bioavailability of at least 15%.

82. The method of claim 58, wherein the oral dissolvable film exhibits a stability of at least about 96% after nine months as measured under 40° C./75% RH accelerated conditions.
